(19) United States
(12) Patent Application Publication
(10) Pub. No.: US 2025/0163454 A1
(43) Pub. Date: May 22, 2025

(54) IN VITRO AND IN VIVO PROTEIN TRANSLATION VIA IN SITU CIRCULARIZED RNAS

(71) Applicant: The Regents of the University of California, Oakland, CA (US)

(72) Inventors: Prashant Mali, La Jolla, CA (US); Aditya Kumar, La Jolla, CA (US); Ester Kwon, La Jolla, CA (US); Katelyn Miyasaki, La Jolla, CA (US)

(21) Appl. No.: 18/835,296

(22) PCT Filed: Feb. 8, 2023
(86) PCT No.: PCT/US2023/062216

§ 371 Date: Aug. 1, 2024

Related U.S. Application Data
(60) Provisional application No. 63/308,309, filed on Feb. 9, 2022.

(51) Int. Cl. C12N 15/85; A61K 39/00; A61K 39/385; A61P 37/04; C12N 15/88

(52) U.S. Cl. CPC C12N 15/85; A61K 39/385; A61P 37/04; C12N 15/88; A61K 2039/53; C12N 2830/48; C12N 2830/50; C12N 2840/203

(57) ABSTRACT

Provided herein are engineered linear RNA polynucleotide molecules that form circular RNA polynucleotide molecules in cells. Also provided are DNA constructs encoding the engineered linear RNA polynucleotide molecules.

### CROSS-REFERENCE TO RELATED APPLICATIONS

This application claims the benefit of U.S. Provisional Application No. 63/308,309 filed Feb. 9, 2022, which is incorporated herein by reference in its entirety.

### STATEMENT OF GOVERNMENT SUPPORT

This invention was made with Government support under Grant Nos. GM123313, CA222826, and HG009285, awarded by the National Institutes of Health; and Grant No. PR210085, awarded by the Department of Defense. The Government has certain rights in the invention.

### SEQUENCE LISTING INCORPORATION

This instant application contains a Sequence Listing, which is been submitted electronically in XML format in accordance to the WIPO Standard ST.26 and is hereby incorporated by reference in its entirety. The XML copy, created on Feb. 8, 2023 is named “00015-411WO1.xml” and is 2,909,290 bytes in size.

### TECHNICAL FIELD

The disclosure provides for ribozyme-mediated constructs and systems and methods thereof, for use in a variety of applications, including for protein production systems, inducible gene expression systems, gene therapy, and combinatorial screening.

### BACKGROUND

Messenger RNA (mRNA) therapeutics have rapidly emerged as viable candidates for the development of vaccines, genome editing, and the treatment of disease. However, the instability of mRNA is a key factor that must be addressed to further improve its clinical relevance. Towards this, mRNA stability has been modulated using a host of approaches, including engineering untranslated regions, incorporation of cap analogs and nucleoside modifications, and codon optimality. More recently, novel circularization strategies, which remove free ends necessary for exonuclease-mediated degradation thereby rendering RNAs resistant to most mechanisms of turnover, have emerged as a particularly promising methodology. However, simple and scalable approaches to achieve efficient in vitro production and purification of circular RNAs are lacking, thus limiting their broader application in research and translational settings.

### SUMMARY

Provided herein is a linearized ribozyme activated RNA construct comprising from 5′ to 3′ end: (a) a first ligation sequence; (b) an IRES sequence; (c) a polynucleotide sequence of interest encoding a recombinant polypeptide; (d) a 3′ UTR sequence; (e) a poly(A) sequence; and (f) a second ligation sequence, wherein the first ligation sequence comprises a 5′-OH end, the second ligation sequence comprises a 2′, 3′-cyclic phosphate end, wherein the first and second ligation sequences form a stem substrate for an RNA ligase.

In some embodiments, the IRES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348), wherein the T nucleotides are U nucleotides in the RNA construct.

In some embodiments, the 3′ UTR sequence is selected from the group consisting of an mtRNR1-AES 3′ UTR (SEQ ID NO: 1354), an mtRNR1-LSP1 3′ UTR (SEQ ID NO: 1355), an AES-mtRNR1 3′ UTR (SEQ ID NO: 1356), an AES-hBg 3′ UTR (SEQ ID NO: 1357), an FCGRT-hBg 3′ UTR (SEQ ID NO: 1358), a 2hBg 3′ UTR (SEQ ID NO: 1359), and a HBA1 3′ UTR (SEQ ID NO: 1360), wherein the T nucleotides are U nucleotides in the RNA construct.

In some embodiments, the 3′ UTR sequence further comprises a WPRE sequence.

In some embodiments, the WPRE sequence comprises the nucleic acid sequence of SEQ ID NO: 1353.

In some embodiments, the poly(A) sequence positioned 3′ of the WPRE sequence.

In some embodiments, the poly(A) sequence has a length ranging from about 5 to about 1000 adenine nucleotides.

In some embodiments, the poly(A) sequence has a length ranging from about 5 to about 300 adenine nucleotides.

In some embodiments, a portion of the first ligation sequence is complementary to a portion of the second ligation sequence.

In some embodiments, the first and/or the second ligation sequence comprises a nucleic acid sequence having at least 90% sequence identity of 5′ AACCAUGCCGACUGAUGGCAG 3′ (SEQ ID NO: 1413).

In some embodiments, the first and/or the second ligation sequence comprises a nucleic acid sequence having at least 90% sequence identity of 5′ CUGCCAUCAGUCGGCGUGGACUGUAG 3′ (SEQ ID NO: 1415).

In some embodiments, the construct lacks a ribozyme.

In some embodiments, the construct comprises one or more modified nucleic acids.

In some embodiments, the construct capable of being introduced into a cell.

In some embodiments, the construct has been introduced into a cell.

Also provided is an engineered cell comprising any one of the linearized ribozyme activated RNA constructs described herein.

In some embodiments, the engineered cell further comprises a circular RNA construct formed from the linearized ribozyme activated RNA construct.

In some embodiments, the cell lacks a DNA construct encoding the linearized ribozyme activated RNA construct.

In some embodiments, the cell is a eukaryotic cell.

In some embodiments, the eukaryotic cell is a mammalian cell.

In some embodiments, the mammalian cell is a human cell.

Provided herein is a method for producing an engineered cell comprising a circular RNA. The method comprises: introducing any one of the linearized ribozyme activated RNA constructs described herein into the cell, wherein an RNA ligase in the cell ligates the first and second ligation sequences, thereby forming the circular RNA construct.

In some embodiments, the RNA ligase is an endogenous RtcB ligase.

In some embodiments, the cell is a eukaryotic cell.

In some embodiments, the eukaryotic cell is a mammalian cell.

In some embodiments, the mammalian cell is a human cell.

Provided herein is a method for producing a circular RNA construct, the method comprises contacting any one of the linearized ribozyme activated RNA constructs described herein with an RNA ligase.

In some embodiments, the contacting is in vitro. In some embodiments, the contacting is inside a cell.

Provided herein is a composition comprising any one of the linearized ribozyme activated RNA constructs described and a delivery system.

In some embodiments, the delivery system comprises any one selected from the group consisting of a lipid nanoparticle, a liposome, a charged polymer, an uncharged polymer, a nanoparticle, a surfactant, a penetrating enhancer, a gene transfer agent, a phospholipid, a micelle, a synthetic vector, a macromolecule, a dendrimer, a biopolymer, a viral particle, and any combination thereof.

In some embodiments, the composition is administered to a subject. In some embodiments, the subject is a human subject.

Also provided herein is a therapeutic composition comprising any one of the linearized ribozyme activated RNA construct described herein and a lipid nanoparticle, wherein the lipid nanoparticle comprises: (6Z,9Z,28Z,31Z)-heptatriaconta-6,9,28,31-tetraen-19-yl-4-(dimethylamino) butanoate (DLin-MC3-DMA); cholesterol; 1,2-distearoyl-sn-glycero-3-phosphocholine (DSPC); and 1,2-dimyristoyl-rac-glycero-3-methoxypolyethylene glycol-2000 (DMG-PEG-2000) at a mole ratio of 50:38.5:10:1.5, respectively, and the lipid nanoparticle has an N/P ratio of 5.4.

In some aspect, provided is a linearized ribozyme-RNA construct comprising from 5′ to 3′ end: (a) a first twister ribozyme; (b) a first ligation sequence; (c) an IRES sequence; (d) a polynucleotide sequence of interest encoding a recombinant polypeptide; (e) a 3′ UTR sequence; (f) a poly(A) sequence; (g) a second ligation sequence; and (h) a second twister ribozyme.

In some embodiments, the IRES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348), wherein the T nucleotides are U nucleotides in the linearized ribozyme-RNA construct.

In some embodiments, the 3′ UTR sequence is selected from the group consisting of an mtRNR1-AES 3′ UTR (SEQ ID NO: 1354), an mtRNR1-LSP1 3′ UTR (SEQ ID NO: 1355), an AES-mtRNR1 3′ UTR (SEQ ID NO: 1356), an AES-hBg 3′ UTR (SEQ ID NO: 1357), an FCGRT-hBg 3′ UTR (SEQ ID NO: 1358), a 2hBg 3′ UTR (SEQ ID NO: 1359), and a HBA1 3′ UTR (SEQ ID NO: 1360), wherein the T nucleotides are U nucleotides in the linearized ribozyme-RNA construct.

In some embodiments, the 3′ UTR sequence further comprises a WPRE sequence.

In some embodiments, the WPRE sequence comprises the nucleic acid sequence of SEQ ID NO: 1353, wherein the T nucleotides are U nucleotides in the linearized ribozyme-RNA construct.

In some embodiments, the poly(A) sequence positioned 3′ of the WPRE sequence.

In some embodiments, the poly(A) sequence has a length ranging from about 5 to about 1000 adenine nucleotides. In some embodiments, the poly(A) sequence has a length ranging from about 5 to about 300 adenine nucleotides.

In some embodiments, the first and/or second ribozyme is selected from the group consisting a twister ribozyme, twister sister (TS) ribozyme, a hammerhead ribozyme, a hairpin ribozyme, a hepatitis delta virus (HDV) ribozyme, a Varkud satellite (VS) ribozyme, a glucosamine-6-phosphate (GimS) ribozyme, a pistol ribozyme, and a hatchet ribozyme. In some embodiments, the first ribozyme and the second ribozyme are the same twister ribozyme. In some embodiments, the first twister ribozyme and/or the second twister ribozyme is a P1 twister ribozyme. In some embodiments, the first ribozyme and/or the second ribozyme is a P3 twister ribozyme.

In some embodiments, the first twister ribozyme and/or the second twister ribozyme comprises a nucleic acid sequence having at least 90% sequence identity of 5′ GCCAUCAGUCGCCGGUCCCAAGCCCGGAUAAAAUGGGAGGGGGCGGGAAACCG CCU 3′ (SEQ ID NO: 1412).

In some embodiments, the first twister ribozyme and/or the second twister ribozyme comprises a nucleic acid sequence having at least 90% sequence identity of 5′ AACACUGCCAAUGCCGGUCCCAAGCCCGGAUAAAAGTGGAGGGUACAGUCCAC GC 3′ (SEQ ID NO: 1414).

In some embodiments, a portion of the first ligation sequence is complementary to a portion of the first twister ribozyme and a portion of the second ligation sequence is complementary to a portion of the second twister ribozyme. In some embodiments, the portion of the first ligation sequence that is complementary to the portion of the first twister ribozyme is also complementary to the portion of the second ligation sequence that is complementary to the portion of the second twister ribozyme.

In some embodiments, the first and/or the second ligation sequence comprises a nucleic acid sequence having at least 90% sequence identity of 5′ AACCAUGCCGACUGAUGGCAG 3′ (SEQ ID NO: 1413). In some embodiments, the first and/or the second ligation sequence comprises a nucleic acid sequence having at least 90% sequence identity of 5′ CUGCCAUCAGUCGGCGUGGACUGUAG 3′ (SEQ ID NO: 1415).

In some embodiments, the construct comprises one or more modified nucleic acids.

Provided herein is a DNA construct comprising a RNA polymerase II promoter and a nucleic acid sequence encoding a ribozyme-RNA construct, wherein the ribozyme-RNA construct comprises from 5′ to 3′ end: (a) a first twister ribozyme; (b) a first ligation sequence; (c) an IRES sequence; (d) a polynucleotide sequence of interest encoding a recombinant polypeptide; (e) a 3′ UTR sequence; (f) a poly(A) sequence; (g) a second ligation sequence; and (h) a second twister ribozyme, wherein promoter is operably linked to the nucleic acid sequence encoding the ribozyme-RNA construct.

In some embodiments, the IRES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348).

In some embodiments, the 3′ UTR sequence is selected from the group consisting of mtRNR1-AES 3′ UTR (SEQ ID NO: 1354), mtRNR1-LSP1 3′ UTR (SEQ ID NO: 1355), AES-mtRNR1 3′ UTR (SEQ ID NO: 1356), AES-hBg 3′ UTR (SEQ ID NO: 1357), FCGRT-hBg 3′ UTR (SEQ ID NO: 1358), 2hBg 3′ UTR (SEQ ID NO: 1359), and HBA1 3′ UTR (SEQ ID NO: 1360).

In some embodiments, the 3′ UTR sequence further comprises a WPRE sequence.

In some embodiments, the WPRE sequence comprises the nucleic acid sequence of SEQ ID NO: 1353.

In some embodiments, the poly(A) sequence is positioned 3′ of the WPRE sequence. In some embodiments, the poly(A) sequence has a length ranging from about 5 to about 1000 adenine nucleotides. In some embodiments, the poly(A) sequence has a length ranging from about 5 to about 300 adenine nucleotides.

In some embodiments, the first and/or second ribozyme is selected from the group consisting a twister ribozyme, twister sister (TS) ribozyme, a hammerhead ribozyme, a hairpin ribozyme, a hepatitis delta virus (HDV) ribozyme, a Varkud satellite (VS) ribozyme, a glucosamine-6-phosphate (GimS) ribozyme, a pistol ribozyme, and a hatchet ribozyme.

The DNA construct of any one of claims 54-62, wherein the first ribozyme and the second ribozyme are the same twister ribozyme. In some embodiments, the first twister ribozyme and/or the second twister ribozyme is a P1 twister ribozyme. In some embodiments, the first ribozyme and/or the second ribozyme is a P3 twister ribozyme.

In some embodiments, the first twister ribozyme and/or the second twister ribozyme comprises a nucleic acid sequence having at least 90% sequence identity of 5′ GCCATCAGTCGCCGGTCCCAAGCCCGGATAAAATGGGAGGGGGCGGGAAACCGC CT 3′ (SEQ ID NO: 1349).

In some embodiments, the first twister ribozyme and/or the second twister ribozyme comprises a nucleic acid sequence having at least 90% sequence identity of 5′ AACACTGCCAATGCCGGTCCCAAGCCCGGATAAAAGTGGAGGGTACAGTCCACG C 3′ (SEQ ID NO: 1350).

In some embodiments, a portion of the first ligation sequence is complementary to a portion of the first twister ribozyme and a portion of the second ligation sequence is complementary to a portion of the second twister ribozyme

In some embodiments, the portion of the first ligation sequence that is complementary to the portion of the first twister ribozyme is complementary to the portion of the second ligation sequence that is complementary to the portion of the second twister ribozyme.

In some embodiments, the first and/or the second ligation sequence comprises a nucleic acid sequence having at least 90% sequence identity of 5′ AACCATGCCGACTGATGGCAG 3′ (SEQ ID NO:1351).

In some embodiments, the first and/or the second ligation sequence comprises a nucleic acid sequence having at least 90% sequence identity of 5′ CTGCCATCAGTCGGCGTGGACTGTAG 3′ (SEQ ID NO: 1352).

In some embodiments, provided herein is A cell comprising any one of the DNA construct described herein.

In some embodiments, cell is a eukaryotic cell. In some embodiments, eukaryotic cell is a mammalian cell. In some embodiments, the eukaryotic cell is a human cell.

Also provided is a cell comprising a circular RNA construct, wherein the circular RNA construct comprises: (a) a first ligation sequence; (b) an IRES sequence positioned 3′ of the first ligation sequence; (c) a polynucleotide sequence of interest encoding a recombinant polypeptide and positioned 3′ of the IRES sequence; (d) a 3′ UTR sequence positioned 3′ of the IRES sequence; (e) a poly(A) sequence positioned 3′ of the 3′ UTR; and (f) a second ligation sequence positioned 3′ of the poly (A) sequence, wherein the first and second ligation sequences are ligated together.

In some embodiments, the cell is a mammalian cell. In some embodiments, the cell is a human cell.

In some embodiments, the cell produces an elevated level of the recombinant polypeptide as compared to a corresponding wild-type cell.

In some embodiments, the first and second ligation sequences are ligated together in the cell by an endogenous RNA ligase.

In some embodiments, the IRES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348), wherein the T nucleotides are U nucleotides in the RNA construct.

In some embodiments, the 3′ UTR sequence is selected from the group consisting of mtRNR1-AES 3′ UTR (SEQ ID NO: 1354), mtRNR1-LSP1 3′ UTR (SEQ ID NO: 1355), AES-mtRNR1 3′ UTR (SEQ ID NO: 1356), AES-hBg 3′ UTR (SEQ ID NO: 1357), FCGRT-hBg 3′ UTR (SEQ ID NO: 1358), 2hBg 3′ UTR (SEQ ID NO: 1359), and HBA1 3′ UTR (SEQ ID NO: 1360), wherein the T nucleotides are U nucleotides in the RNA construct.

In some embodiments, the 3′ UTR sequence comprises a WPRE sequence. In some embodiments, the WPRE sequence comprises the nucleic acid sequence of SEQ ID NO: 1353, wherein the T nucleotides are U nucleotides in the RNA construct.

In some embodiments, the poly(A) sequence is positioned 3′ of the WPRE sequence.

In some embodiments, the poly(A) sequence has a length ranging from about 5 to about 1000 adenine nucleotides. In some embodiments, the poly(A) sequence has a length ranging from about 5 to about 300 adenine nucleotides.

In some embodiments, a portion of the first ligation sequence is complementary to a portion of the second ligation sequence.

In some embodiments, the first and/or the second ligation sequence comprises a nucleic acid sequence having at least 90% sequence identity of 5′ AACCAUGCCGACUGAUGGCAG 3′ (SEQ ID NO: 1413).

In some embodiments, the first and/or the second ligation sequence comprises a nucleic acid sequence having at least 90% sequence identity of 5′ CUGCCAUCAGUCGGCGUGGACUGUAG 3′ (SEQ ID NO: 1415).

In some embodiments, the circular RNA construct comprises one or more modified nucleic acids.

In some embodiments, the cell is an engineered cell.

In some embodiments, the cell lacks any one of the DNA constructs described herein and lacks any one of the linearized ribozyme-RNA construct described herein.

Provided herein is a ribozyme RNA-construct(s) comprising from 5′ to 3′: an optional primer region, an optional barcode region, a first ribozyme domain, a first ligation stem domain, a payload domain, a second ligation stem domain, and a second ribozyme domain; wherein the payload domain comprises from 5′ to 3′: an internal ribosome entry site (IRES) or a P2A peptide coding sequence, a coding sequence of at least one polypeptide and/or nucleic acid of interest, and a 3′UTR sequence; wherein the transcription of the payload domain is activated by or dependent upon the activity of the one or more ribozymes.

In some embodiments, the first and second ligation stem domains are from 30 to 60 bp in length. In some embodiments, the first and second ligation stem domains are from 40 to 50 bp in length.

In some embodiments, the first and second ribozymes are selected from the group consisting of a twister ribozyme, a hammerhead ribozyme, a hatchet ribozyme, a hepatitis delta virus ribozyme, a ligase ribozyme, a pistol ribozyme, a twister sister ribozyme, a Vg1 ribozyme, a VS ribozyme and derivatives of any of the foregoing.

In some embodiments, the first and second ribozymes are twister ribozymes.

In some embodiments, the first ribozyme is a P3 twister ribozyme.

In some embodiments, the second ribozyme is a P1 twister ribozyme.

In some embodiments, the first ligation stem domain comprises a 5′-OH end, the second ligation stem domain comprises a 2′, 3′-cyclic phosphate end, and wherein the first and second ligation stem domains form a stem substrate for an RNA ligase.

In some embodiments, the RNA ligase is RtcB.

In some embodiments, the payload or the at least one polypeptide of interest comprises a zinc finger or CRISPR-Cas9 coding sequence. In a further embodiment, the payload comprises a polypeptide having endonuclease activity and comprises a sequence having at least 85% sequence identity (e.g., 85%, 87%, 90%, 92%, 95%, 98%, 99% or 100%) to SEQ ID NO:1439 and has a mutation selected from the group consisting of L513T, L622Q, and a combination of L513T and L622Q, wherein the polypeptide can perform editing activity (e.g., endonuclease activity) with CRISPR In another embodiment, the Cas9 variant has a sequence of SEQ ID NO:1439 with 1-10 (e.g., 1, 2, 3, 4, 5, 6, 7, 8, 9, or 10) conservative amino acid substitutions and has an L513T, L622Q or an L513T and L622Q mutation, wherein the polypeptide can perform editing activity (e.g., endonuclease activity) with CRISPR

In another embodiment, the payload domain or the at least one polypeptide of interest comprise a sequence that encodes a polypeptide/protein selected from insulin, clotting factor IX, the cystic fibrosis transmembrane conductance regulator protein, and the dystrophin protein.

In some embodiments, the ribozyme RNA-construct(s) is linearized.

In some embodiments, the 3′ UTR comprises a Woodchuck Hepatitis Virus Postranslational Regulatory Element (WPRE).

In some embodiments, the WPRE is followed by a poly(A) stretch.

In some embodiments, the 3′ UTR sequence is selected from the group consisting of an mtRNR1-AES 3′ UTR (SEQ ID NO: 1354), an mtRNR1-LSP1 3′ UTR (SEQ ID NO: 1355), an AES-mtRNR1 3′ UTR (SEQ ID NO: 1356), an AES-hBg 3′ UTR (SEQ ID NO: 1357), an FCGRT-hBg 3′ UTR (SEQ ID NO: 1358), a 2hBg 3′ UTR (SEQ ID NO: 1359), and a HBA1 3′ UTR (SEQ ID NO: 1360), wherein the T nucleotides are U nucleotides in the RNA construct.

In some embodiments, the first and/or the second ligation stem domain comprises a nucleic acid sequence having at least 90% sequence identity of 5′ AACCAUGCCGACUGAUGGCAG 3′ (SEQ ID NO: 1413).

In some embodiments, the first and/or the second ligation stem domain comprises a nucleic acid sequence having at least 90% sequence identity of 5′ CUGCCAUCAGUCGGCGUGGACUGUAG 3′ (SEQ ID NO: 1415).

In some embodiments, the WPRE sequence comprises the nucleic acid sequence of SEQ ID NO: 1353.

In some embodiments, the RES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348), wherein the T nucleotides are U nucleotides in the RNA construct.

In some embodiments, a vector or plasmid comprises any one of the ribozyme RNA-construct(s) described located downstream of an RNA promoter.

In some embodiments, the RNA promoter is a polymerase III promoter.

In some embodiments, the polymerase III promoter is a hU6 promoter.

In some embodiments, the first and second ligation stem domains are substrates of naturally occurring ligases in situ. In some embodiments, the naturally occurring ligase is RtcB.

In some embodiments, at least one polypeptide of interest comprises two or more polypeptides of interest separated by a self-cleaving peptide.

In some embodiments, the self-cleaving peptide comprises a 2A- or 2A-like-peptide.

In some embodiments, at least one polypeptide of interest is selected from the group consisting of a prodrug activating enzyme, a biological response modifier, a receptor ligand, an immunoglobulin derived binding polypeptide, a non-immunoglobulin binding polypeptide, an antigenic polypeptide, a genome editing enzyme, and any combination thereof wherein multiple polypeptides are separated by a 2A or 2A-like peptide.

In some embodiments, biological response modifier or an immunopotentiating cytokine.

In some embodiments, the immunopotentiating cytokine is selected from the group consisting of interleukins 1 through 38, interferon, tumor necrosis factor (TNF), and granulocyte-macrophage-colony stimulating factor (GM-CSF).

In some embodiments, the 2A- or 2A-like peptide further comprises a GSG linker moiety.

In some embodiments, the genome editing enzyme is selected from the group consisting of a zinc finger nuclease, a transcription activator-like effector nuclease (TALEN), an engineered meganuclease and an RNA-guided DNA endonuclease (Cas) polypeptide.

In some embodiments, the 5′ and 3′ ribozyme sequences are independently selected from a sequence that is at least 85-100% identical to 5′-GCCATCAGTCGCCGGTCCCAAGCCCGGATAAAATGGGAGGGGGCGGGAAACCGC CT-3′ (SEQ ID NO:1349) or 5′-AACACTGCCAATGCCGGTCCCAAGCCCGGATAAAAGTGGAGGGTACAGTCCACG C-3′ (SEQ ID NO:1350), wherein T can be U.

In some embodiments, the first and second ligation stem domains are independently selected from a sequence that is at least 85-100% identical to 5′-AACCATGCCGACTGATGGCAG-3′ (SEQ ID NO:1351) or 5′-CTGCCATCAGTCGGCGTGGACTGTAG-3′ (SEQ ID NO:1352).

Also provided is an RNA or DNA vector comprising any one of the ribozyme RNA-construct(s) or any one of the DNA constructs described herein.

In some embodiments, the vector is a viral vector. In some embodiments, the viral vector is a replicating or non-replicating retroviral vector. In some embodiments, the viral vector is an adenoviral vector, an adeno-associated viral vector (AAV), or a lentiviral vector. In some embodiments, the adenoviral vector is selected from the group consisting of AAV1 serotype, an AAV2 serotype, AAV3 serotype, an AAV4 serotype, AAV5 serotype, an AAV6 serotype, AAV7 serotype, an AAV8 serotype, an AAV9 serotype, a derivative of any of these.

Provided herein is a circular RNA construct obtain by in vitro transcription of any one of the DNA constructs or any one of the ribozyme activated RNA-constructs described herein.

In some embodiments, the construct comprises a duplex of the first and second ligation stem domains and (i) an internal ribosome entry site (IRES) or a P2A peptide coding sequence, (ii) a coding sequence of at least one polypeptide and/or nucleic acid of interest, and (iii) a 3′UTR sequence.

In some embodiments, the at least one polypeptide of interest is selected from the group consisting of a prodrug activating enzyme, a biological response modifier, a receptor ligand, an immunoglobulin derived binding polypeptide, a non-immunoglobulin binding polypeptide, an antigenic polypeptide, a genome editing enzyme, and any combination thereof wherein multiple polypeptides are separated by a 2A or 2A-like peptide.

In some embodiments, the circular RNA construct comprises a coding region for a gene editing polypeptide and a nucleic acid guide sequence.

Also provided is a pharmaceutical composition comprising any one of the RNA constructs, any one of the vectors or any one of the circular RNA constructs described herein, and a pharmaceutically acceptable carrier.

Also provided is a host cell comprising any one of the RNA constructs, any one of the vectors, or any one of the circular RNA constructs described herein.

In some embodiments, the host cell is a eukaryotic cell.

In some embodiments, the ribozyme RNA construct, the vector or the circular RNA construct is episomal.

In some embodiments, the circular RNA constructs edits the genome or an expressed RNA in the host cell.

Also provided is a vaccine composition comprising any one of the ribozyme RNA-construct(s) described herein, wherein the ribozyme RNA-construct(s) is linearized and comprises: a 5′ ribozyme; a 5′ ligation sequence; an internal ribosome entry site (IRES) sequence; an RNA coding sequence for at least one antigenic polypeptide; a 3′UTR sequence; a 3′ ligation sequence; and a 3′ ribozyme sequence, and a pharmaceutically acceptable carrier.

Provided is a vaccine composition comprising the RNA construct described herein, wherein the coding sequence encoding a polypeptide of interest encodes for an antigenic polypeptide.

Also provided is a polypeptide having improved CRISPR-Cas editing efficiency. The polypeptide has improved Cas9 editing efficiency compared to the wildtype Cas9 of SEQ ID NO:1439. The polypeptide has at least 85% sequence identity (e.g., 85%, 87%, 90%, 92%, 95%, 98%, 99% or 100%) to SEQ ID NO:1439 and has a mutation selected from the group consisting of L513T, L622Q, and a combination of L513T and L622Q, wherein the polypeptide can perform editing activity (e.g., endonuclease activity) with CRISPR In a further embodiment, the polypeptide can have 5-6 additional mutations selected from the group consisting of (i) Y285Q, L726G, L815D, L1244G and L1281A; and (b) Y285Q, S367C, L726G, L815D, L1244G and L1281A. In another embodiment, the polypeptide has a sequence of SEQ ID NO:1439 with 1-10 (e.g., 1, 2, 3, 4, 5, 6, 7, 8, 9, or 10) conservative amino acid substitutions and has an L513T, L622Q or an L513T and L622Q mutation, wherein the polypeptide can perform editing activity (e.g., endonuclease activity) with CRISPR. In a further embodiment, the polypeptide can have 5-6 additional mutations selected from the group consisting of (i) Y285Q, L726G, L815D, L1244G and L1281A; and (b) Y285Q, S367C, L726G, L815D, L1244G and L1281A. Additional Cas9 mutants are provided in FIGS. 5B and 14B (e.g., mutant V3, V4 and V5).

The disclosure also provides a method of treating a subject with a genetic mutation comprising contacting the subject with a icRNA comprising a sequence encoding a CRISPR-Cas9 or variant thereof. In one embodiment, the Cas9 variant comprises a sequence that has at least 85% sequence identity (e.g., 85%, 87%, 90%, 92%, 95%, 98%, 99% or 100%) to SEQ ID NO:1439 and has a mutation selected from the group consisting of L513T, L622Q, and a combination of L513T and L622Q, wherein the polypeptide can perform editing activity (e.g., endonuclease activity) with CRISPR In a further embodiment, the polypeptide can have 5-6 additional mutations selected from the group consisting of (i) Y285Q, L726G, L815D, L 1244G and L 1281A; and (b) Y285Q, S367C, L726G, L815D, L 1244G and L 1281A. In another embodiment, the Cas9 variant has a sequence of SEQ ID NO:1439 with 1-10 (e.g., 1, 2, 3, 4, 5, 6, 7, 8, 9, or 10) conservative amino acid substitutions and has an L513T, L622Q or an L513T and L622Q mutation, wherein the polypeptide can perform editing activity (e.g., endonuclease activity) with CRISPR In a further embodiment, the polypeptide can have 5-6 additional mutations selected from the group consisting of (i) Y285Q, L726G, L815D, L 1244G and L 1281A; and (b) Y285Q, S367C, L726G, L815D, L 1244G and L 1281A.

Utilizing work on autocatalytic RNA circularization, circular RNAs for programmable RNA editing have been engineered and previously described. The primary approach for generating these circular guide RNAs was via delivery of encoding DNA molecules where the guide RNAs were expressed using pol-III promoters, and thereby were both generated and circularized in cells. However, in vitro transcribed RNAs delivered in linear form successfully circularize in situ in cells upon entry and were similarly functional as guide RNAs. Motivated by the simplicity of this latter approach, and its compatibility with routine in vitro synthesis and purification processes, it was explored if Since this latter approach is more simplistic and is compatible with routine in vitro synthesis and purification processes, this framework was used to generate circular messenger RNAs, which, as described herein, showed that engineered in situ gircularized RNAs (icRNAs) enable extensive protein translation. Data provided herein demonstrate the versatility of icRNAs (such as linear forms of RNA molecules that circularize in situ in cells and the resulting in situ circularized RNA molecules) via a range of in vitro and in vivo applications spanning from RNA vaccines to genome and epigenome targeting.

### DESCRIPTION OF DRAWINGS

FIGS. 1A-1E show robust protein translation via icRNAs, and application to RNA vaccines and genome editing. (a) Schematic describing the production of icRNAs. In this embodiment, the icRNAs are generated via in vitro transcription of linear RNAs that bear a twister ribozyme flanked by, e.g., internal ribosome entry site (IRES) coupled to a messenger RNA of interest (e.g., a payload comprising an IRES, coding sequence, and 3′UTR). Once transcribed (e.g., into a linear IVT RNA), the flanking twister ribozymes rapidly self-cleave, enabling hybridization of the complementary ligation stems to one another, and upon delivery into cells, these linear RNAs (also referred to as linear icRNA herein) are then circularized in situ by the ubiquitous RNA ligase RtcB to produce the circular icRNA. In the in situ circularization form, translation of the payload occurs, therefore expressing the protein encoded by the payload. (b) GFP mRNA expression over time for cells transfected with linear icRNA (delivered in a linear format but then circularized once delivered into a cell to generate circular icRNA in the cell) and linear in situ Circularization #effective RNAs (linear icdRNAs) is shown. HEK293T cells were transfected with GFP icRNA (delivered in a linear format icRNA but then circularized once delivered into a cell to produce circular icRNA; circle) and linear icdRNA (square) and GFP mRNA amount was measured over time. The 6-hour time point was included to assess initial RNA input (left panel, n=3, p=0.414; t-test, two-tailed). Data from days 1, 2, and 3 illustrate persistence of icRNA (middle panel). Values represented as mean±SEM (n=3, p=0.000143 for day 1, p<0.0001 for day 2, p<0.0001 for day 3 t-test, two-tailed). Values were normalized to the 6-hour time point. GFP protein expression was largely gone by day 3 in linear icdRNA (right panels). (c) RT-PCR based confirmation of circularization of the linear icRNA in cells by detection of circular icRNA. (d) Lipid nanoparticles containing linear icRNA or linear icdRNA in which the payload for both was the COVID Omicron spike were injected intramuscularly into Balb/c mice. After 21 days, sera was isolated from mice and IgG antibody production against the spike protein was quantified by ELISA. Values represented as mean+/−SEM (n=6, p=0.0005 for 2 μg icRNA compared to 2 μg icdRNA; one-way ANOVA, post-hoc Tukey test). (e) Editing efficiency of zinc finger nucleases targeting the GFP and CCR5 genes delivered as a linear icRNA and linear icdRNA. Values represented as mean+/−SEM (n=3, p=0.0051 for GFP and p<0.0001 for CCR5; unpaired t-test, two tailed).

FIGS. 2A-2G show LORAX protein engineering methodology to screen progressively de-immunized Cas9 variants, and their delivery to a cell as linear icRNAs to generate circular icRNAs in the cell. (a) Schematic of the LORAX protein engineering methodology. A lentiviral library containing Cas9 variants and a sgRNA targeting the HPRT1 gene are delivered into HeLa cells. After treatment with 6TG to remove cells containing non-functional variants, genomic DNA is isolated and Cas9 variant sequences are amplified. Sequences are then read using long-range nanopore sequencing to identify hits. (b) Library construction schematic where mutated epitopes across the Cas9 backbone are identified. The Cas9 sequence was broken up into smaller blocks, such that each block contained no more than 3 or 4 epitopes. The variant library was then assembled by mixing these blocks at equal concentration, generating variants that contained mutated epitopes throughout the sequence. (c) Replicate correlation of post-screen library element frequencies. (d) Network reconstruction connecting Cas9 variants with similar mutational patterns. Circles in red represent tested variants and labeled with their respective names. (e) HEK293T cells were transduced with the GFP coding sequence disrupted by the insertion of a stop codon and a 68-bp genomic fragment of the AAVS1 locus. Wildtype (WT) or Cas 9 variants, a sgRNA targeting the AAVS1 locus, and a donor plasmid capable of restoring GFP function via homology directed repair (HDR) were introduced (transfected) into the cell line and flow cytometry was performed on day 3 to validate Cas9 variants identified in the screen via relative quantification of GFP expression restored by HDR. Values represented as mean±SEM (n=3). (f) Linear icRNA for Cas9 wildtype or variant V4, along with a sgRNA targeting the AAVS1 locus, was introduced into HEK293T and K562 cells, in which these linear icRNA were circularized to produce corresponding circular icRNAs in these cells. Editing efficiency at the AAVS1 locus in the two cell lines are plotted. Values represented as mean±SEM (n=3). (g) Linear icRNA for CRISPRoff wildtype or variant V4, along with a sgRNA targeting the B2M gene, were introduced into HEK293T cells, wherein the linear icRNA circularized in the HEK293T cells to produce circular icRNA. B2M gene repression of CRISPRoff constructs in the presence or absence of sgRNA was plotted. Values represented as mean±SEM (n=3).

FIGS. 3A-3C provides engineering of the icRNA to improve translation, in vivo delivery, and circularization of icRNA upon delivery to a cell. (a) a panel of IRES sequences, ligation stems, and 3′ untranslated regions (UTRs) (sequences of FIGS. 17A-17C) were screened to optimize protein translation (b) Characterization of lipid nanoparticles (LNPs) encapsulating linear icRNA by dynamic light scattering. No differences in size were observed for LNPs containing circular icRNA (solid line) or linear icdRNA (dotted line). (c) LNPs containing either circular icRNA (circle) or linear icdRNA (square) were retro orbitally injected into C57BL/6J mice, in which the linear icRNA circularized to produce circular icRNA in cells after injection, and RNA was isolated from livers on days 3 and 7. mRNA expression from circular icRNA or linear icdRNA at days 3 and 7, and RT-PCR based confirmation of delivered linear icRNA circularization in vivo to produce the circular icRNA. The ratio of circular RNA detected normalized to icRNA day 3 expression was plotted (n=3). RT-PCR confirmed circular icRNA persistence.

FIGS. 4A-4D presents screening results from the LORAX method. (a) Immunogenicity scores for Cas orthologs, demonstrating reduced immunogenicity as the number of mutated epitopes increase. (b) Presence of HPRT1 converts 6TG into a toxic byproduct. HeLa cells transduced with wildtype Cas9 and either a HPRT1 targeting or nontargeting (NTC) guide. Only cells where the HPRT1 gene is disrupted are capable of living in various concentrations of 6TG. 6 μg/mL 6TG was used for the screen due to no cells being present in the NTC group. (c) Variant Cas9 sequences were amplified from the plasmid library or genomic DNA post-screen. Long-read nanopore sequencing was performed and the mutational density distribution for the predicted library, the constructed Cas9 variant library, and the two replicates post-screen are plotted. (d) Cas9 block composition and pre- and post-screen allele frequencies at each of the 18 mutational sites. Each block and site shows enrichment of the wild-type allele, but all sites retain a substantial fraction of mutant alleles.

FIGS. 5A-5C presents functional validation of progressively de-immunized Cas9 variants identified using LORAX. (a) Correlation between the fold change of a Cas9 variant and neighboring variants that share similar mutational patterns. It shows the correlation between the fold change of a Cas9 variant and its predicted fold-change based on a k-nearest neighbors regression. Neighboring variants are those that share similar mutational patterns. The strong correlation suggests a smooth fitness landscape in which variants with similar mutation patterns will be more similar in fitness, on average, than those with divergent mutation patterns. (b) Cas9 wildtype or variants V1-20 and sgRNA targeting the AAVS1 locus were introduced into HEK293T cells. NHEJ mediated editing efficiency of the AAVS1 locus was quantified via NGS for Cas9 WT and variants V1-20 is plotted and the accompanying mutational profiles for all variants is included. Variant genotypes are listed in the lower panel. (c) Predicted reduction in immunogenicity based on the epitope mutated and the HLA typing is depicted for each mutation included in the screen.

FIGS. 6A-6C presents further characterization of genome and epigenome editing capable with Cas9 variants V3, V4, and V5. (a) Cas9 wildtype or variants V3, V4, or V5, along with sgRNAs targeting the respective genes, were introduced into HEK293T and K562 cells. Editing efficiency of variants across 4 additional genes in HEK293T and 5 gene loci in K562 is plotted. (b) ASCL1 mRNA expression in cells transfected with dCas9 WT-VPR or dCas9 V4-VPR and sgRNA or no sgRNA is shown. Values represented as mean±SEM (n=3). (c) CXCR4 mRNA expression for cells transfected with dCas9 WT-KRAB or dCas9 V4-KRAB and sgRNA or no sgRNA. Values represented as mean±SEM (n=3).

FIGS. 7A-7G show engineering and characterization of icRNAs. (a) Schematic describing the production of icRNAs. These are generated via in vitro transcription of linear RNAs that bear a twister ribozyme flanked internal ribosome entry site (IRES) coupled to a messenger RNA of interest and a 3′UTR. Once transcribed, the flanking twister ribozymes rapidly self-cleave, enabling hybridization of the complementary ligation stems to one another, and upon delivery into cells, these linear RNAs (also referred to herein as linear icRNA) are then circularized in situ by the ubiquitous RNA ligase RtcB to produce a circular RNA (also referred to herein as a circular icRNA). After in situ circularization, translation of the payload occurs, therefore expressing the protein encoded by the payload. (b) HEK293T cells were transfected with GFP icRNA (delivered in a linear icRNA but then circularized once delivered into a cell to produce circular icRNA; circle) and linear icdRNA (square) and GFP mRNA amount was measured over time. The 6-hour time point was included to assess initial RNA input (left panel, n=3, p=0.414; t-test, two-tailed). Data from days 1, 2, and 3 illustrate persistence of icRNA (middle panel). Values represented as mean±SEM (n=3, p=0.000143 for day 1, p<0.0001 for day 2, p<0.0001 for day 3 t-test, two-tailed). Values were normalized to the 6-hour time point. GFP protein expression was largely gone by day 3 in linear icdRNA transfected cells (right panel). (c) RT-PCR based confirmation of linear icRNA in situ circularization in cells to produce the detected circular icRNA. (d) HEK293 Ts were transfected with linear icRNAs containing various IRES sequences, which were circularized after transfection to generate circular icRNA, and then GFP intensity was quantified by flow cytometry. Circular icRNAs containing the encephalomyocarditis virus IRES (the third bar from the right, corresponding to x-axis label 17) and coxsackievirus B3 IRES (the bar farthest to the right, corresponding to x-axis label 19) were selected for further engineering. Values represented as mean±SEM (n=3). (e) HEK293 Ts were transfected with linear icRNAs containing the encephalomyocarditis virus IRES coupled with various 3′ untranslated regions, and poly(A) stretches (blue bars, #3-13), which were circularized after transfection to generate circular icRNA, and GFP intensity was quantified by flow cytometry. Addition of a Woodchuck Hepatitis Virus Posttranscriptional Regulatory Element (WPRE) and a poly(A) stretch significantly improved protein translation, and icRNAs bearing these and either EMCV IRES or CVB3 IRES (green bar, #14) were used for all subsequent studies. These designs were also compared with IRES and 3′ untranslated regions from Chen et al. (gray bars, #15-16), and commercially capped linear RNA produced by Trilink Biotechnologies (red bar, #17). Values represented as mean±SEM (n=3). (f) HEK293 Ts were transfected with linear icRNA (for circularization after transfection to produce circular icRNA in the HEK293 Ts) or pre-circularized icRNA, and RNA was isolated at various time points. The left panel shows a schematic of delivery of (1) a linear icRNA into a cell such that the icRNA undergoes in situ circularization to form a circular icRNA and (2) a linear icRNA undergoing in vitro circularization to form a pre-circ RNA. The pre-circ RNA can be delivered into a cell. RT-PCR was performed and the ratio of the circular icRNA band to pre-circularized icRNA band was plotted to evaluate in situ circularization efficiencies. The relative circularization efficiency % is shown in the bar graph—data for EMCV IRES containing icRNA and pre-circ RNA is represented in the left bar for each timepoint) or CVB3 IRES containing icRNA and pre-circ RNA is represented in the (right bar for each timepoint). Values represented as mean±SEM (n=3). (g) Sanger sequencing trace mapping the junction site formed upon ligation of the left (underlined in brown; 10 nucleotides on left of trace map) and right (underlined in red; 5 nucleotides on right of trace map) stems.

FIGS. 8A-8E show assessment of circular icRNA persistence in in vitro and in vivo settings. (a) Post differentiation of stem cells into cardiomyocytes, linear icRNA (circular icRNA was generated in the cardiomyocytes after transfection) or linear commercial RNA were transfected into cells and images were taken over three weeks. Normalized GFP expression over time was plotted for circular icRNA comprising an EMCV IRES, GFP, WPRE, and 165 poly(A) stretch (“EMCV”; see top data line), circular icRNA comprising an CVB3 IRES, GFP, WPRE, and 165 poly(A) stretch (“CVB3”; see middle data line), and linear RNA comprising GFP (“Linear”; see bottom data line). Values represented as mean±SEM (n=3). (b) Representative images are shown illustrating circular icRNA persistence in cardiomyocytes from part (a). (c) Lipid nanoparticles containing linear icRNA comprising either the EMCV IRES or CVB3 IRES and either further comprising 5% N6-methyladenosine (m6A) modification if present (“EMCV m6A” or “CBV3 m6A”) or not comprising 5% N6-methyladenosine (m6A) modification (“EMCV unmodified” or “CBV3 unmodified”) (circular icRNA was generated from these linear icRNAs in cells after delivery) or linear mRNA, each encoding for human erythropoietin (EPO), were retro orbitally injected into C57BL/6J mice and serum was isolated from mice on day 1, 2, 3, 4, and 7. EPO protein expression was assessed by ELISA. Expression normalized to day 1 for each respective group is plotted (n=3). In the graph at day 2, the bottom data point show “Linear”, the middle data points show “EMCV unmodified” and “CBV3 unmodified”, and the top data points show “EMCV m6A” and “CBV3 m6A”. (d) EPO RNA expression in the liver after 7 days normalized to EMCV circular icRNA was quantified by qPCR and plotted (n=3). (e) RT-PCR confirmed in vivo circularization of linear EMCV icRNA constructs and linear CVB3 icRNA constructs to produce circular EMCV icRNA and circular CVB3 icRNA (farthest right two lanes).

FIGS. 9A-9C show application of icRNAs to zinc finger mediated genome targeting. Editing efficiency of circular icRNA or linear icdRNA zinc finger nucleases targeting a stably integrated GFP gene or the endogenous CCR5 gene in HEK293T cells is plotted. Values represented as mean±SEM (n=3). (b) Repression efficiency of zinc finger KRAB proteins produced by circular icRNA in HeLa cells is plotted on the left as hPCSK9 expression fold change relative to the circular GFP icRNA quantified with RT-qPCR after 48 hours. The green (bottom) dotted line indicates the measure for successful repression of hPCSK9 by a human zinc finger KRAB protein. hPCSK9 expression fold change of concurrent delivery of three human zinc finger KRAB proteins (28_R, 131_R, and 402; n=3) delivered as circular icRNAs relative to a circular GFP icRNA control (n=3) is plotted on the right (p=0.0127; t-test, two-tailed). (c) Repression efficiency in vivo of three screened mouse zinc finger KRAB proteins delivered concurrently as circular icRNA retro orbitally injected into C57BL/6J mice is plotted on the right (n=3, p=0.056; t-test) quantified by RT-qPCR on RNA extracted from livers.

FIG. 10 shows the LORAX protein engineering methodology to screen progressively deimmunized Cas9 variants. (Left panel: library design) Low-frequency SNPs that have limited effect on Cas9 function were identified and immunogenicity was evaluated in silico using the netMHC epitope prediction software to identify candidate mutations. This analysis was performed for many Cas9 orthologs. Mutations were generated such that two bps were changed to account for Nanopore sequencing accuracy. A library was then generated by fusion PCR of blocks containing wildtype and mutations at specific epitopes. Location of epitopes in SpCas9 that were combinatorially mutated and screened is shown. (Right panel: library screen) The screen was performed by transducing HeLa cells with a lentiviral library containing the Cas9 variants and a guide that cuts the HPRT1 gene. HPRT1 knockout produces resistance to 6-TG. After two weeks, DNA is extracted from surviving cells, Cas9 variant sequences are PCR amplified from the genomic DNA and Nanopore sequenced. High accuracy of variant identification is possible due to the use of two bp mutations for each amino acid change. Post-screen library element frequencies across two independent replicates is shown. Replicate correlation was calculated excluding the over-represented wild-type sequence.

FIGS. 11A-11F show validation of LORAX screen identified Cas9 variants for de-immunization, and genome and epigenome targeting via delivery as linear icRNAs that subsequently circularize to generate circular icRNAs in cells. (a) Network reconstruction connecting Cas9 variants with similar mutational patterns. Node colors indicate the number of deimmunized epitopes (dark blue <3, light blue=3, white=4, yellow=5, pink >5). Circles in red represent tested variants and labeled with their respective names. (b) HEK293T bearing a GFP coding sequence disrupted by the insertion of a stop codon and a 68-bp genomic fragment of the AAVS1 locus were used as a reporter line. Wildtype (WT) or Cas9 variants, a sgRNA targeting the AAVS1 locus, and a donor plasmid capable of restoring GFP function via homology directed repair (HDR) were transfected into these cells and flow cytometry was performed on day 3. Relative quantification of GFP expression restoration by HDR is plotted. The number in parentheses represents the number of mutations in the variant. Values represented as mean±SEM (n=3). (c) T2 cells were pulsed with wildtype and variant peptides, cultured with PBMCs, and an ELISpot assay was performed to assess PBMC IFN-γ secretion to wildtype and variant peptides. Number of spot forming colonies for each peptide is plotted (n=3, mean±SEM, *p<0.05, **p<0.01, unpaired t-test two-tailed). Red letters in the peptide sequences represent the mutated amino acid. (d) RNA encoding for Cas9 wildtype or variant V4 were electroporated into PBMCs to assess the whole protein immunogenicity. ELISpot assay was performed to assess PBMC IFN-γ secretion to wildtype and variant protein. Number of spot forming colonies for each peptide is plotted (n=3, mean±SEM, ****p<0.0001, unpaired t-test two-tailed). (e) Linear icRNA for Cas9 wildtype or variant V4, along with a sgRNA targeting the AAVS1 locus, were introduced into HEK293T and K562 cells, in which these linear icRNA were circularized to produce corresponding circular icRNAs in these cells. Editing efficiency at the AAVS1 locus in the two cell lines are plotted. Values represented as mean±SEM (n=3). (f) Linear icRNA for CRISPRoff wildtype or variant V4, along with a sgRNA targeting the B2M gene, were introduced into HEK293T cells and subsequently circularized in the HEK293T cells to produce circular icRNA. B2M gene repression of CRISPRoff constructs in the presence or absence of sgRNA is plotted. Values represented as mean±SEM (n=3).

FIGS. 12A-12E show persistence and immunogenicity of circular icRNAs in vitro and in vivo. (a) HEK293T cells were transfected with linear GFP icRNA containing encephalomyocarditis IRES with WPRE and 50 nt poly(A) stretch (“IRES_ECMV+WPRE+pA50”; circle) or commercial 5′ capped linear RNA (“Trilink”; square), wherein theses linear GFP icRNAs were circularized to generate circular GFP icRNAs in the HEK293T cells, and then the GFP mRNA amount was measured over time (left axis). Values were normalized to the amount at the 6 hour time point for each respective group (n=3, p=0.0072 for day 1, p=0.0015 for day 2, p=0.00086 for day 3, p=0.0037 for day 4, and p=0.000531 for day 5; t-test, two-tailed). The ratio of circular icRNA GFP mRNA compared to linear commercial RNA for each day is plotted (dashed line (“Ratio”); right axis). The increase in value over time illustrates improved persistence of circular icRNA. (b) Characterization of lipid nanoparticles (LNPs) encapsulating linear icRNA by dynamic light scattering. No differences in size were observed for LNPs containing linear icRNA (solid line) or linear icdRNA (dotted line). (c) LNPs containing either linear icRNA (circle) or linear icdRNA (square) were retro orbitally injected into C57BL/6J mice, in which the linear icRNA circularized to produce circular icRNA in cells after injection, and RNA was isolated from livers on days 3 and 7. The ratio of circular RNA detected normalized to icRNA day 3 expression was plotted (n=3). RT-PCR confirmed circular icRNA persistence up to day 7 in vivo. (d) HeLa cells were transfected with modified (5% m6a) and unmodified circular GFP icRNA containing encephalomyocarditis IRES with WPRE and 165 nt poly(A) stretch and 5′ capped modified (pseudo-UTP) and unmodified linear GFP RNA and IL-6, IFNB, and RIG-I mRNA quantities measured via RT-qPCR after 24 hours. For IL-6 data from left to right of the graph, linear unmodified icRNA is in first bar, linear modified icRNA is in second bar, circular unmodified icRNA is in third bar, circular modified icRNA is in fourth bar and untreated cells is in fifth bar. IFNB and RIG-1 data is shown in a similar order. Values were normalized to untreated HeLa samples (n=3, IL-6: p=0.014, 0.014, 0.021, 0.040 respectively, IFNB: p=0.040 for unmodified linear GFP, and n.s. for all other comparisons; 1% FDR, t-test, two-tailed). Trend illustrates a minimal immunogenic response to all constructs except for unmodified linear RNA. Values represented as mean±SEM. (e) LNPs containing modified (pseudo-UTP) linear GFP RNA and modified circular GFP icRNA were retro orbitally injected into C57BL/6J mice, RNA was isolated from livers on day 3, and IL-6 and RIG-I mRNA quantities measured via RT-qPCR. The expression fold change of modified circular GFP icRNA was plotted normalized to modified linear GFP RNA (n=3, p=0.41 for IL-6, p=0.26 for RIG-I; 1% FDR, t-test, two-tailed). Modified linear GFP RNA data is in left bar and modified circular GFP icRNA data is in right bar for IL-6, and also similarly shown for RIG-1. Values represented as mean±SEM.

FIGS. 13A-13D show the LORAX screen design and results. (a) Immunogenicity scores for Cas orthologs, demonstrating reduced immunogenicity (averaged across HLA types) as the number of mutated epitopes increases. (b) Presence of HPRT1 converts 6TG into a toxic nucleotide analog. HeLa cells transduced with wildtype Cas9 and either a HPRT1 targeting or nontargeting (NTC) guide. Only cells where the HPRT1 gene is disrupted are capable of living in various concentrations of 6TG. 6 μg/mL 6TG was used for the screen as this concentration was sufficient for complete killing of NTC-bearing cells. (c) Variant Cas9 sequences were amplified from the plasmid library or genomic DNA post-screen. Long-read nanopore sequencing was performed and the mutational density distribution for the predicted library, the constructed Cas9 variant library, and the two replicates post-screen are plotted. At 0 mutations along the x-axis, “theoretical” is at bottom, “plasmid” is at middle, and replicate 1 and replicate 2 are at top of the frequency axis. (d) Cas9 block composition and pre- and post-screen allele frequencies at each of the 18 mutational sites. Each block and site shows enrichment of the wild-type allele, but all sites retain a substantial fraction of mutant alleles.

FIGS. 14A-14B show validations of LORAX screen identified Cas9 variants. (a) Correlation between the fold change of a Cas9 variant and its predicted fold-change based on a k-nearest neighbors regression. Neighboring variants are those that share similar mutational patterns. The strong correlation suggests a smooth fitness landscape in which variants with similar mutation patterns will be more similar in fitness, on average, than those with divergent mutation patterns. (b) Cas9 wildtype or variants V1-20 and sgRNA targeting the AAVS1 locus were introduced into HEK293T cells. NHEJ mediated editing at the AAVS1 locus was quantified via NGS for Cas9 WT and variants V1-20 is plotted. The number in parentheses represents the number of mutations in the variant. Variant genotypes are listed in the lower panel.

FIGS. 15A-15C show predicted and experimentally confirmed deimmunization across Cas9 epitopes. (a) Predicted mutation-specific reduction in immunogenicity based on the epitope mutated and the HLA typing is depicted for each mutation included in the screen. (b) Technical replicates of spot forming colonies in the ELISpot assay to assess peptide epitope immunogenicity are plotted for each donor (n=4). (c) Technical replicates of spot forming colonies in the ELISpot assay to assess whole protein immunogenicity are plotted for each donor (n=6).

FIGS. 16A-16D show characterization of Cas9 variants across genome and epigenome targeting assays. (a) Cas9 wild-type or variants V3, V4, or V5, along with sgRNAs targeting the respective genes, were introduced into HEK293T and K562 cells. Editing efficiency of variants across 4 loci in HEK293 Ts and 5 loci in K562s is plotted. WT represented by circle, V3 represented by square, V4 represented by triangle, and V5 represented by diamond. (b) ASCL1 mRNA expression in cells transfected with dCas9 WT-VPR or dCas9 V4-VPR and sgRNA or no sgRNA is shown. Values represented as mean±SEM (n=3). (c) CXCR4 mRNA expression in cells transfected with dCas9 WT-KRAB or dCas9 V4-KRAB and sgRNA or no sgRNA is shown. Values represented as mean±SEM (n=3). (d) Volcano plot demonstrating differentially expressed genes for CRISPRoff wildtype with or without the B2M guide and variant V4 with or without guide. Dotted lines indicate the cutoff for significance (log₂(fold change) greater than 0.5 or less than −0.5 and −log₁₀ p-value greater than 3). B2M downregulation is confirmed by the red dots. Differentially expressed genes found in both wildtype and V4 are labeled with purple dots.

FIGS. 17A-17C show exemplary embodiments of DNA constructs encoding for icRNA constructs comprising IRES sequences, ligation stem sequences, and 3′ untranslated regions (UTRs). (a) depicts the nucleic acid sequence of a UTR version 1 construct from 5′ to 3′: a T7 promoter, a 5′ twister ribozyme, a 5′ ligation stem sequence, an internal ribosome entry site (IRES), an mRNA of interest (e.g., a GFP payload), a WPRE, a 4 deoxyadenosine stretch (linker), a 3′ ligation stem sequence, a 3′ twister ribozyme, and a stop (poly-T) sequence. (b) depicts the DNA sequence of a UTR version 2 construct from 5′ to 3′: a T7 promoter, a 5′ twister ribozyme, a 5′ ligation stem sequence, an IRES, an mRNA of interest (e.g., a GFP payload), a 4 deoxyadenosine stretch (linker), a 3′ ligation stem, a 3′ twister ribozyme, and a stop (poly-T) sequence. (c) depicts the nucleic acid sequence of a UTR version 3 construct from 5′ to 3′: a T7 promoter, a 5′ twister ribozyme, a 5′ ligation stem sequence, an IRES, an mRNA of interest (e.g., a GFP payload), a WPRE, a poly-A stretch (such as a 31 deoxyadenosine stretch), a 3′ ligation stem sequence, a 3′ twister ribozyme, and a stop (poly-T) sequence. Note that slashes indicate boundaries of the components (e.g., elements, features, and motifs).

FIGS. 18A-18S show exemplary embodiments of DNA constructs encoding for icRNA constructs comprising the different IRES constructs of Table 1. (a) depicts a cricket paralysis virus (CrPV) IRES construct (SEQ ID NO: 1361). (b) depicts a Homo sapiens IGF2 virus IRES construct (SEQ ID NO: 1362). (c) depicts a hepatovirus A (HAV) IRES construct (SEQ ID NO: 1363). (d) depicts a hepatitis C virus H77 isolate (HCV-H77) IRES construct (SEQ ID NO: 1364). (e) depicts a Homo sapiens FGF1 IRES construct (SEQ ID NO: 1365). (f) depicts a bovine viral diarrhea virus 1 (BVDV-1; also known as pestivirus A) IRES construct (SEQ ID NO: 1366). (g) depicts a human rhinovirus A89 (RV-A89) IRES construct (SEQ ID NO: 1367). (h) depicts a pan paniscus LIMA1 IRES construct (SEQ ID NO: 1368). (i) depicts a human adenovirus 2 (hAdV2) IRES construct (SEQ ID NO: 1369). (j) depicts a Montana myotis leukoencephalitis Virus (MMLV) IRES construct (SEQ ID NO:1370). (k) depicts a Homo sapiens RANBP3 IRES construct (SEQ ID NO: 1371). (l) depicts a pestivirus giraffe 1 IRES construct (SEQ ID NO: 1372). (m) depicts a Homo sapiens TGIF1 IRES construct (SEQ ID NO: 1373). (n) depicts a human poliovirus 1 Mahoney IRES construct (SEQ ID NO: 1374). (o) depicts a foot and mouth disease virus type O (FMDV O) IRES construct (SEQ ID NO: 1375). (p) depicts an encephalomyocarditis virus 7A (EMCV 7A) IRES construct (SEQ ID NO: 1376). (q) depicts an encephalomyocarditis virus 6A (EMCV 6A) IRES construct (SEQ ID NO:1377). (r) depicts an enterovirus 71 (EV71) IRES construct (SEQ ID NO: 1378). (s) depicts a coxsackievirus B3 (CVB3) IRES construct (SEQ ID NO: 1379). The ribozyme sequences and ligation sequences are italicized. The IRES sequences are underlined. The sequences of the polynucleotide encoding the payload (e.g., encoding GFP) are bolded. Note that slashes indicate boundaries of the components (e.g., elements, features, and motifs). Data shown in FIG. 7D are of the Table 1 IRES constructs corresponding to the sequences in FIGS. 18A-18S, which respectively correspond to construct #1-19 of the FIG. 7D.

FIGS. 19A-19N show exemplary embodiments of DNA constructs encoding for icRNA constructs of Table 2 that have different 3′ UTR constructs. (a) depicts an IRES_Encephalomyocarditis Virus 6A+WPRE construct (SEQ ID NO: 1380). (b) depicts an IRES_Encephalomyocarditis Virus 6A+WPRE+pA50 3′construct (SEQ ID NO: 1381). (c) depicts an IRES Encephalomyocarditis Virus 6A+WPRE+pA120 3′construct (SEQ ID NO: 1382). (d) depicts an IRES Encephalomyocarditis Virus 6A+WPRE+pA165 3′construct (SEQ ID NO: 1383). (e) depicts an IRES Encephalomyocarditis Virus 6A+WPRE+pA165 3′+mtRNR1-AES 3′ UTR construct (SEQ ID NO: 1384). (f) depicts an IRES Encephalomyocarditis Virus 6A+WPRE+pA165 3′+mtRNR1-LSP1 3′ UTR construct (SEQ ID NO: 1385). (g) depicts an IRES Encephalomyocarditis Virus 6A+WPRE+pA165 3′+AES-mtRNR1 3′ UTR construct (SEQ ID NO: 1386). (h) depicts an IRES Encephalomyocarditis Virus 6A+WPRE+pA165 3′+AES-hBg 3′ UTR construct (SEQ ID NO: 1387). (i) depicts an IRES_Encephalomyocarditis Virus 6A+WPRE+pA165 3′+FCGRT-hBg 3′ UTR construct (SEQ ID NO: 1388). (j) depicts an IRES Encephalomyocarditis Virus 6A+WPRE+pA165 3′+2hBg 3′ UTR construct (SEQ ID NO: 1389). (k) depicts an IRES Encephalomyocarditis Virus 6A+WPRE+pA165 3′+HBA1 3′ UTR construct (SEQ ID NO: 1390). (l) depicts an IRES Coxsackievirus B3+WPRE+pA165 3′ (SEQ ID NO: 1391). (m) depicts an icChang AES-mtRNR1 construct (SEQ ID NO: 1392). (n) depicts an icChang HBA1 construct (SEQ ID NO: 1393). The ribozyme sequences and ligation sequences are italicized. The IRES sequences are underlined. The WPRE sequences are double underlined. The poly(A) stretches are italicized and underlined. The 3′ UTR sequences are italicized and double underlined. The sequences of the polynucleotide encoding the payload (e.g., encoding GFP) are bolded. The nucleic acid sequences encoding poly-A binding proteins are italicized and underlined. Note that slashes indicate boundaries of the components (e.g., elements, features, and motifs). Data shown in FIG. 7E are of the 3′ UTR constructs of Table 2 corresponding to the sequences in FIGS. 19A-19N, in which correspond to construct #3-16 of the FIG. 7E (construct #1 and 2 of FIG. 7E correspond to the encephalomyocarditis virus 7A (EMCV 7A) IRES construct (SEQ ID NO:1376) and the encephalomyocarditis virus 6A (EMCV 6A) IRES construct (SEQ ID NO:1377) in Table 1).

### DETAILED DESCRIPTION

### Definitions

As used herein and in the appended claims, the singular forms “a,” “an,” and “the” include plural referents unless the context clearly dictates otherwise. Thus, for example, reference to “a prodrug” includes a plurality of such prodrugs and reference to “the chemotherapeutic agent” includes reference to one or more chemotherapeutic agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

RNAs are a powerful therapeutic class. However their inherent transience impacts their activity both as an interacting moiety as well as a template. Circularization of RNA improves their persistence, however simple and scalable approaches to achieve this are lacking. Utilizing autocatalytic RNA circularization, linear in situ circularized RNAs (icRNAs) were engineered and are described herein. This approach enables icRNA delivery as simple linear RNA that can be circularized upon delivery into the cell, thus making them compatible with routine synthesis, purification, and delivery formulations. Protein translation from circularized icRNAs was confirmed both in vitro and in vivo and their utility in three contexts is demonstrated herein: (1) the SARS-CoV-2 Omicron spike protein was delivered in vivo as linear icRNAs and showed corresponding induction of humoral immune responses; (2) robust genome targeting via zinc finger nucleases delivered as linear icRNAs was demonstrated; and (3) to enable compatibility between persistence of expression and immunogenicity, a long range multiplexed (LORAX) protein engineering methodology was developed to screen progressively deimmunized Cas9 proteins, and demonstrated efficient genome and epigenome targeting via their delivery as icRNAs.

The terms “administration” and “administering” refer to the act of giving a drug, prodrug, or other agent, or therapeutic treatment (e.g., DNA or RNA polynucleotide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “alphavirus” has its conventional meaning in the art, and includes the various species such as Venezuelan Equine Encephalitis (VEE) Virus, Eastern Equine Encephalitis (EEE) virus, Everglades Virus (EVE), Mucambo Virus (MUC), Pixuna Virus (PIX), and Western Equine Encephalitis Virus, all of which are members of the VEEEEE Group of alphaviruses. Other alphaviruses include, e.g., Semliki Forest Virus (SFV), Sindbis, Ross River Virus, Chikungunya Virus, S.A. AR86, Barmah Forest Virus, Middleburg Virus, O'nyong-nyong Virus, Getah Virus, Sagiyama Virus, Bebaru Virus, Mayaro Virus, Una Virus, Aura Virus, Whataroa Virus, Banbanki Virus, Kyzylagach Virus, Highlands J Virus, Fort Morgan Virus, Ndumu Virus, and Buggy Creek Virus. Alphaviruses particularly useful in the constructs and methods described herein are VEE/EEE group alphaviruses.

The terms “alphavirus RNA replicon”, “alphavirus replicon RNA”, “alphavirus RNA vector replicon”, “vector replicon RNA” and “self-replicating RNA construct” are used interchangeably to refer to an RNA molecule expressing nonstructural protein genes such that it can direct its own replication (amplification) and comprises, at a minimum, 5′ and 3′ alphavirus replication recognition sequences, coding sequences for alphavirus nonstructural proteins, and a polyadenylation tract. It may additionally contain one or more elements (e.g., IRES sequences, 2A peptide sequence and the like) to direct the expression, meaning transcription and translation, of a coding sequence of interest. The alphavirus replicon of the disclosure can comprise, in one embodiment, 5′ and 3′ alphavirus replication recognition sequences, coding sequences for alphavirus nonstructural proteins, a polyadenylation tract.

The term “adeno-associated virus” or “AAV” as used herein refers to a member of the class of viruses associated with this name and belonging to the genus depend parvovirus, family Parvoviridae. Multiple serotypes of this virus can be suitable for gene delivery. In some cases, serotypes can infect cells from various tissue types. Examples of AAV serotypes are AAV1, AAV2, AAV3, AAV4, AAV5, AAV6, AAV7, AAV8, AAV9, AAV10, and AAV11. Non-limiting exemplary serotypes useful for the purposes disclosed herein include any of the 11 serotypes, e.g., AAV2 and AAV8.

As used herein, the term “circularized” and/or “circular” used in the context of a nucleic acid molecule (e.g., an engineered guide RNA) can generally refer to a nucleic acid molecule that can be represented as a polynucleotide sequence in a circular 2-dimensional format with one nucleotide after the other wherein the represented polynucleotide is circular or a closed loop. In some embodiments, a circular nucleic acid molecule does not comprise a 5′ reducing hydroxyl, a 3′ reducing hydroxyl, or both capable of being exposed to a solvent.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that may not be designed to be complementary to or can be only partially complementary to any other nucleic acid sequence.

The term “encode” as it is applied to polynucleotides can refer to a polynucleotide which is said to “encode” a polypeptide if, in its native state or when manipulated, it can be transcribed and/or translated to produce the mRNA for the polypeptide and/or a fragment thereof. The antisense strand is the complement of such a nucleic acid, and the encoding sequence can be deduced therefrom. In a non-limiting example, an RNA molecule can encode a polypeptide during translation, whereas a DNA molecule can encode an RNA molecule during transcription. The term “encode” also includes the expression of a nucleic acid that when expressed has a biological effect (e.g., guide RNA, antisense molecule, siRNA and the like).

The terms “equivalent” or “biological equivalent” are used interchangeably when referring to a particular molecule, biological or cellular material having minimal homology while still maintaining desired structure or functionality.

As used herein, “expression” can refer to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

“Homology” or “identity” or “similarity” can refer to sequence similarity between two peptides or between two nucleic acid molecules. Homology can be determined by comparing a position in each sequence which can be aligned for purposes of comparison. For example, when a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the disclosure.

Homology can refer to a percent (%) identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein, such particular peptide, polypeptide or nucleic acid sequence can be determined conventionally using computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

For example, in a specific embodiment the identity between a reference sequence (query sequence, a sequence of the disclosure) and a subject sequence, also referred to as a global sequence alignment, can be determined using the FASTDB computer program. In some cases, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity can be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

The term “hybridization” as used herein refers to the complementary base-pairing interaction of one nucleic acid with another nucleic acid that results in formation of a duplex, triplex, or other higher-ordered structure. In some instances, the event, state, or process includes two complementary single-stranded RNA molecules or portions thereof bonded together to form a double-stranded complex. The term “self-hybridization” as used herein refers to an event or state in which a nucleic acid strand is hybridized to itself, such as hybridized to a portion of itself.

The term “hybridization” also includes an event, state, or process in which one or more polynucleotides react to form a complex that is stabilized via hydrogen bonding between the bases of the nucleotide residues. The hydrogen bonding can occur by Watson-Crick base pairing, Hoogstein binding, or in any other sequence-specific manner. A hybridization reaction can constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The terms “increased”, “increase”, “enhanced”, “enhance”, “elevate”, or “elevated” are all used herein to generally mean an increase by a statically significant amount. In some embodiments, the terms refer to an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “isolated” as used herein can refer to molecules or biologicals or cellular materials being substantially free from other materials. In one aspect, the term “isolated” can refer to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also can refer to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and may not be found in the natural state. In some cases, the term “isolated” is also used herein to refer to polypeptides which are isolated from other cellular proteins and is meant to encompass both purified and recombinant polypeptides. In some cases, the term “isolated” is also used herein to refer to cells or tissues that are isolated from other cells or tissues and is meant to encompass both cultured and engineered cells, or tissues.

A “ligation sequence”, “ligation stem”, or “ligation stem domain” are used interchangeably herein and refers to a nucleic acid sequence complementary to another nucleic acid sequence, which enables the formation of Watson-Crick base pairing to form suitable substrates for ligation by a ligase, e.g., an RNA ligase. In one embodiment, a 5′ ligation sequence and a 3′ ligation sequence are substrates for an RNA ligase such as, but not limited to RtcB. The 5′ and 3′ ligation sequences when ligated circularize an RNA molecule of the disclosure. Such circularization reduces RNA degradation and improves persistence in vivo.

As used herein, a “linearized ribozyme activated RNA construct” refers to a construct of the disclosure which has been activated by the activity of ribozymes. In contrast, a “linear ribozyme RNA construct” refers to an RNA construct prior to ribozyme activation. The difference can be identified via the presence of at least the ribozymes present on the RNA construct. For example, a linearized ribozyme activated RNA construct lacks ribozymes, while an unactivated construct includes at least one ribozyme domain.

“Operably linked” refers to an arrangement of elements where the components so described are configured so as to perform their usual function. Thus, control sequences (e.g., a promoter, enhancer and the like) operably linked to a coding sequence are capable of effecting the transcription, and in some cases, the translation, of a coding sequence. The control sequences need not be contiguous with the coding sequence so long as they function to direct the expression of the coding sequence. Similarly, and internal ribosome entry site can be “operably linked” to a downstream coding sequence such that the coding sequence is properly expressed.

The terms “polynucleotide” and “oligonucleotide” are used interchangeably and refer to a polymeric form of nucleotides of any length, either deoxyribonucleotides or ribonucleotides or analogs or combinations thereof. Polynucleotides can have any three-dimensional structure and can perform any function. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also can refer to both double and single stranded molecules. Unless otherwise specified or required, any embodiment of this disclosure that is a polynucleotide can encompass both the double stranded form and each of two complementary single stranded forms known or predicted to make up the double stranded form. In some embodiments, a polynucleotide can include both RNA and DNA nucleotides.

The term “polynucleotide sequence” can be the alphabetical representation of a polynucleotide molecule. This alphabetical representation can be input into databases in a computer having a central processing unit and used for bioinformatics applications such as functional genomics and homology searching. In any alphabetic representation, the disclosure contemplates both RNA and DNA (wherein “T” is replaced with “U” or vice-a-versa).

The term “pharmaceutical composition” refers to the combination of an active ingredient with a carrier, inert or active, making the composition especially suitable for therapeutic, prophylactic, or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

The term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethyl sulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21th Ed., MackPubl. Co., Easton, Pa. (2005), incorporated herein by reference in its entirety.

In certain embodiments, “promoters” may be used to drive transcription of an operably linked nucleic acid. As used herein “promoter” refers to a DNA sequence which contains the binding site for RNA polymerase and initiates transcription of a downstream nucleic acid sequence. A promoter for use in the disclosure can be a constitutive, inducible or tissue specific, or a temporal promoter. Suitable promoters can be derived from viruses, prokaryotes and eukaryotes. Suitable promoters can be used to drive expression by any RNA polymerase. Examples of inducible promoters include, but are not limited to, T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, and the like. As used herein, “promoter” includes pol-III promoters such as H1 and U6 promoters. Inducible promoters can be regulated by various molecules such as doxycycline. In one embodiment, the promoter is a prokaryotic promoter selected from the group consisting of T7, T3, SP6 and derivatives thereof.

As used herein, a “ribozyme” (ribonucleic acid enzyme) is an RNA molecule capable of catalyzing biochemical reactions. A “self-cleaving ribozyme” is a ribozyme capable of cleaving itself. The ribozyme used in the disclosure can be any small endonucleolytic ribozyme that will self-cleave in the target cell type including, for example, hammerhead, hairpin, the hepatitis delta virus, the Varkud satellite, twister, twister sister, pistol and hatchet. See, e.g., Roth et al., Nat Chem Biol. 10(1):56-60; and Weinberg et al., Nat Chem Biol. 2015 August; 11(8):606-10, both incorporated herein by reference. U.S. 2015/0056174 provides modified hammerhead ribozymes with enhanced endonucleolytic activity. Ribozymes cleave the substrate RNA in a sequence specific manner at a substrate cleavage site. Typically, a ribozyme contains a catalytic region flanked by two binding regions. The ribozyme binding regions hybridize to the substrate RNA, while the catalytic region cleaves the substrate RNA at a substrate cleavage site to yield a cleaved RNA product. In various embodiment, the 5′ or 3′ of various constructs can be a twister ribozyme or a twister sister ribozyme. For example, the 5′ and 3′ ribozymes of various constructs are either a P3 or P1 twister ribozyme but not both P3 or both P1.

The term “subject” broadly refers to any animal, including but not limited to, human and non-human animals (e.g., dogs, cats, cows, horses, sheep, pigs, poultry, fish, crustaceans, etc.).

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, CA), LIPOFECTAMINE® (Invitrogen), FUGENE® (Roche Applied Science, Basel, Switzerland), JETPEI™ (Polyplus-transfection Inc., New York, NY), EFFECTENE® (Qiagen, Valencia, CA), DREAMFECT™ (OZ Biosciences, France) and the like), or electroporation. Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals. Standard recombinant DNA and molecular cloning techniques used herein are well known in the art and are described in Sambrook, J., Fritsch, E. F. and Maniatis, T., Molecular Cloning: A Laboratory Manual, 2^{nd}ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety. Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

As used herein, the term “vector” can refer to a nucleic acid construct or lipid or molecule designed for transfer between different hosts or medium, including but not limited to a plasmid, a virus, a cosmid, a phage, a bacterial artificial chromosome (BAC), a yeast artificial chromosome (YAC), etc. In some embodiments, a “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves. Alphavirus vectors, such as Semliki Forest virus-based vectors and Sindbis virus-based vectors, have also been developed for use in gene therapy and immunotherapy. In aspects where gene transfer is mediated by a retroviral vector, a vector construct can refer to the polynucleotide comprising the retroviral genome or part thereof, and a gene of interest. Methods for the introduction of vectors or constructs into cells are known to those of skill in the art and include, but are not limited to, lipid-mediated transfer (i.e., liposomes, including neutral and cationic lipids), electroporation, nucleofection, direct injection, cell fusion, particle bombardment, calcium phosphate co-precipitation, DEAE-dextran-mediated transfer and/or viral vector-mediated transfer.

The terms “treat”, “treating” and “treatment”, as used herein, refers to ameliorating symptoms associated with a disease or disorder. Also, the terms “treat”, “treating” and “treatment” include preventing or delaying the onset of the disease or disorder symptoms, and/or lessening the severity or frequency of symptoms of the disease or disorder.

The disclosure demonstrates engineered in situ circularized RNAs (icRNAs). This approach enables icRNA delivery as linear RNA (linear icRNA), thus making them compatible with routine laboratory synthesis, purification, and delivery formulations. The disclosure demonstrates protein translation and persistence from circular icRNAs both in vitro and in vivo, and confirmed their versatility and activity in applications spanning from potential regenerative medicine applications to genome and epigenome targeting. Notably, the icRNA strategy provided for generation and delivery of large constructs, such as CRISPRoff, which would be more cumbersome to deploy via lentiviral and adeno-associated virus (AAVs) due to packaging limits.

The disclosure also provides a LORAX platform of protein engineering that can be applied iteratively to tackle particularly challenging multiplexed protein engineering tasks by exploring huge swaths of combinatorial mutation space unapproachable using previous techniques. The technique was used to create a Cas9 variant with seven simultaneously deimmunized epitopes which still retains robust functionality in a single round of screening. The platform provides for gene editing to long-persistence therapeutic modalities such as AAV or icRNA delivery. Furthermore, while this methodology is particularly suited to the unique challenges of protein deimmunization, it is also applicable to any potential protein engineering goal, so long as there exists an appropriate screening procedure to select for the desired functionality.

The versatility of the LORAX platform can be modified to includes protein structure, coevolutionary epistatic constraints, amino acid signaling motifs, or T-/B-cell receptor binding repertoires, among other possibilities. The disclosure provides a network-based method for differentiating spurious from bonafide hits leveraging known aspects of protein epistasis and fitness landscapes. Similar customizations and tweaks relevant to the specific biology of a given problem may yield substantial returns in applying LORAX or other large-scale combinatorial screening methods to various protein engineering challenges.

Using the LORAX platform Cas9 immunotolerant polypeptide were developed. The disclosure a Cas9 variant comprises a sequence that has at least 85% sequence identity (e.g., 85%, 87%, 90%, 92%, 95%, 98%, 99% or 100%) to SEQ ID NO:1439 and has a mutation selected from the group consisting of L513T, L622Q, and a combination of L513T and L622Q, wherein the polypeptide can perform editing activity (e.g., endonuclease activity) with CRISPR In a further embodiment, the polypeptide can have 5-6 additional mutations selected from the group consisting of (i) Y285Q, L726G, L815D, L1244G and L 1281A; and (b) Y285Q, S367C, L726G, L815D, L 1244G and L 1281A. In another embodiment, the Cas9 variant has a sequence of SEQ ID NO:1439 with 1-10 (e.g., 1, 2, 3, 4, 5, 6, 7, 8, 9, or 10) conservative amino acid substitutions and has an L513T, L622Q or an L513T and L622Q mutation, wherein the polypeptide can perform editing activity (e.g., endonuclease activity) with CRISPR In a further embodiment, the polypeptide can have 5-6 additional mutations selected from the group consisting of (i) Y285Q, L726G, L815D, L 1244G and L 1281A; and (b) Y285Q, S367C, L726G, L815D, L 1244G and L 1281A.

Disclosed herein are in situ gircularized RNAs (icRNAs) engineered utilizing autocatalytic RNA circularization (sometimes referred to as inducible ribozyme-mediated RNA-construct system). This approach enables icRNA delivery as a linear RNA polynucleotide (e.g., molecule) or a linear ribozyme-activated RNA construct. icRNAs are compatible with routine laboratory synthesis, purification, and delivery formulations. Uses of icRNAs in both in vitro and in vivo methods provided increased protein translation and persistence. In some embodiments, an icRNA comprises different combinations of regulatory domains (e.g., IRESs) and 3′ domains (e.g., 3′ UTRs) for increased protein translation. Their versatility and activity in applications spanning from gene therapy and RNA vaccines to genome and epigenome targeting are described herein.

A circular RNA polynucleotide (also referred to as a circular ribozyme-activated RNA construct) can be generated by the circularization of a linear RNA polynucleotide comprising ligation sequences that can be ligated together by an RtcB protein, thereby producing the circular RNA polynucleotide. The linear RNA polynucleotide (also referred to as a hybridization construct where the ligation sequences hybridize to each other; a linearized ribozyme activated RNA polynucleotide; linearized ribozyme activated RNA construct; a linear icRNA; or a linear version of the circular icRNA, which are used interchangeably) is a substrate for the RtcB enzyme.

In some embodiments, the linearized ribozyme activated RNA construct (linear icRNA) includes one or more components selected from the group consisting of: two ligation sequences, an IRES or self-cleaving peptide coding sequence, a WPRE or a 3′ UTR sequence with an optional poly(A) sequence, and a polynucleotide of interest. In some embodiments, the linearized ribozyme activated RNA construct includes one or more components selected from the group consisting of: two hybridized ligation sequences, an IRES, a WPRE, a 3′ UTR sequence, a poly(A), and a polynucleotide of interest (e.g., encoding a payload or transgene). In some embodiments, a spacer or linker sequence is present between any components of the linearized ribozyme activated RNA construct. In some embodiments, the polynucleotide of interest encodes a polypeptide of interest such as, but not limited to, a fill-length protein, a fusion protein, a chimeric protein, a recombinant protein, a therapeutic protein, a protein fragment, a truncated protein, and the like.

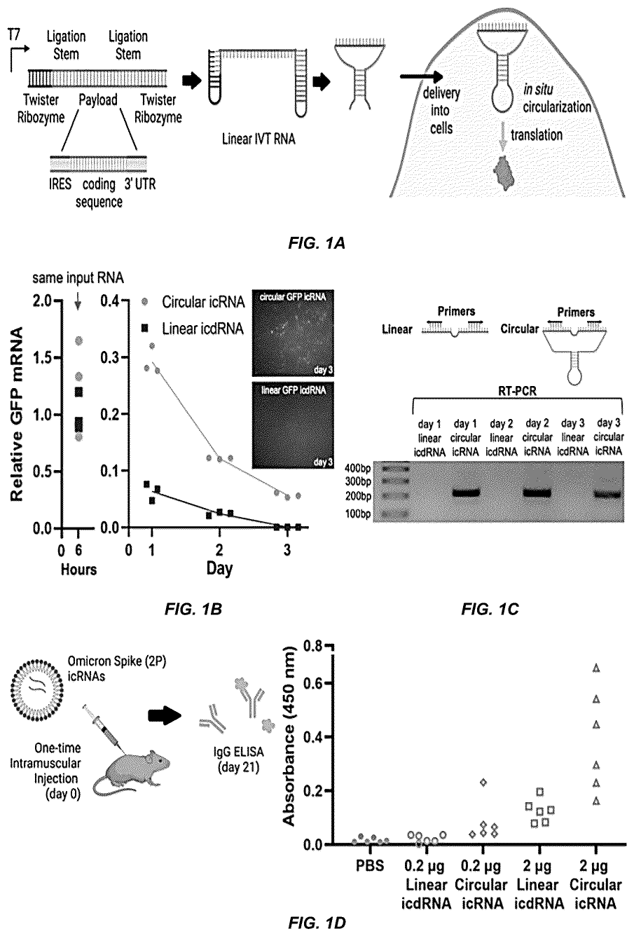

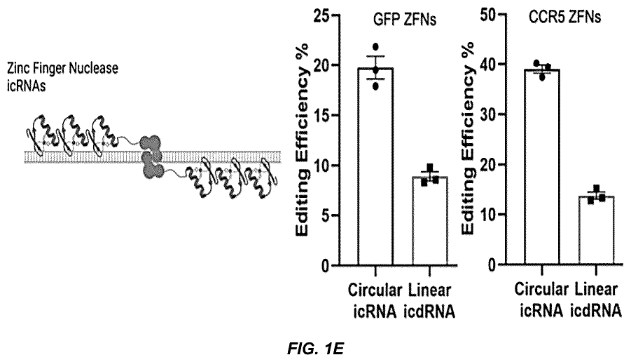

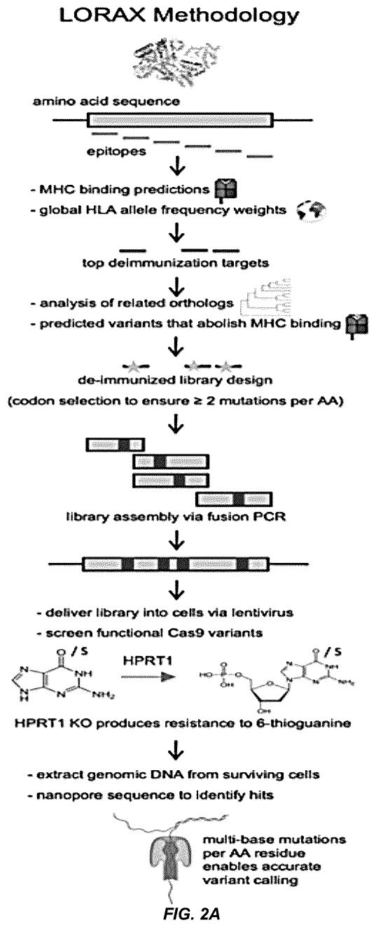

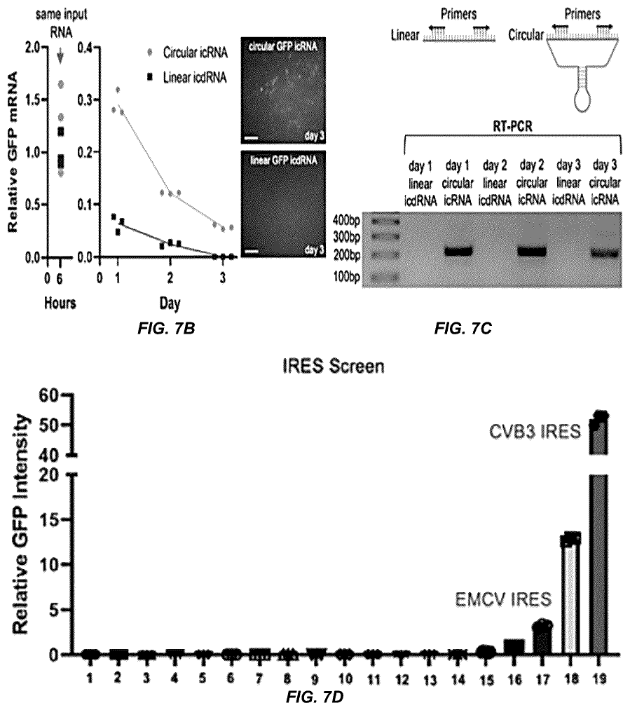

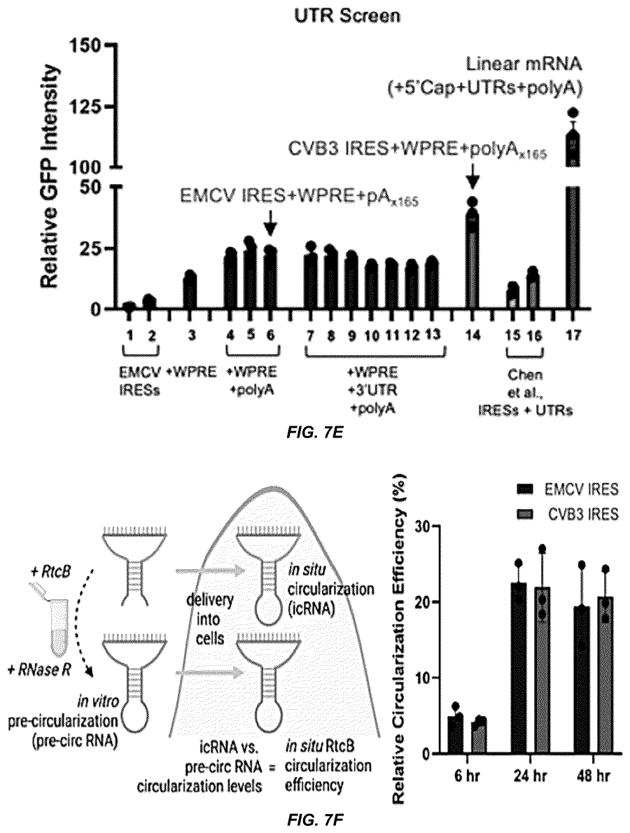

In various embodiments, the linearized ribozyme activated RNA construct comprises in 5′ to 3′ order: a ligation sequence (e.g., 5′ ligation sequence), a regulatory sequence (e.g., an IRES sequence and the like), a polynucleotide sequence of interest, a WPRE sequence, a poly(A) sequence, and a ligation sequence (e.g., 3′ ligation sequence). In some embodiments, the linearized ribozyme activated RNA construct includes in 5′ to 3′ order: a ligation sequence (e.g., 5′ ligation sequence), an IRES sequence, a polynucleotide sequence of interest, a WPRE sequence or a 3′ UTR sequence, and an optional poly(A) sequence, and a ligation sequence (e.g., 3′ ligation sequence). The complementary 5′ ligation sequence and 3′ ligation sequence can hybridize together and the linear RNA polynucleotide can form a stem structure (e.g., a ligation stem structure). See, FIGS. 1A, 7A, and 7G for an example of a linearized ribozyme activated RNA construct with a stem structure (in particular, the RNA polynucleotide shown immediately upstream of the action of delivery into cells).

In some embodiments, the ligation sequences are located at opposite ends of the linearized ribozyme activated RNA construct and one ligation sequence includes a 5′ hydroxyl end and the other ligation sequence includes a 2′,3′-cyclic phosphate end. In some embodiments, the ligation sequences are or include sections that are complementary to each other. In some embodiments, the ligation sequences are at least partially complementary to each other. In some embodiments, the ligation sequences or a portion thereof hybridize together in a cell or in standard in vitro conditions. In some embodiments, the ligation sequences are at least 85%, e.g., at least 85%, 86%, 87%, 88%, 89%, 90%, 91%, 92%, 93%, 94%, 95%, 96%, 97%, 98%, 99% or more complementary over the length of one of the sequences. In some embodiments, the ligation sequences are 100% complementary over the length of one of the sequences. In some instances, the 5′ ligation sequence is at least 85%, e.g., at least 85%, 86%, 87%, 88%, 89%, 90%, 91%, 92%, 93%, 94%, 95%, 96%, 97%, 98%, 99% or more complementary to the 3′ ligation sequence. In various instances, the 3′ ligation sequence is at least 85%, e.g., at least 85%, 86%, 87%, 88%, 89%, 90%, 91%, 92%, 93%, 94%, 95%, 96%, 97%, 98%, 99% or more complementary to the 5′ ligation sequence. In some embodiments, a linear RNA polynucleotide hybridizes to itself (e.g., self-hybridizes) at the 5′ and 3′ ligation sequences.

In some embodiments, the 5′ ligation sequence or the 3′ ligation sequence comprises the nucleic acid sequence of SEQ ID NO: 1413 (5′-AACCAUGCCGACUGAUGGCAG-3′). In some embodiments, the 5′ ligation sequence or the 3′ ligation sequence comprises a nucleic acid sequence having at least 90%, e.g., at least 90%, 91%, 92%, 93%, 94%, 95%, 96%, 97%, 98%, 99% or more sequence identity to SEQ ID NO: 1413. In some cases, the 5′ and 3′ ligation sequences both comprise the nucleic acid sequence of SEQ ID NO: 1413. In several embodiments, the 5′ ligation sequence or the 3′ ligation sequence comprises the nucleic acid sequence of SEQ ID NO: 1415 (5′-CUGCCAUCAGUCGGCGUGGACUGUAG-3′). In various embodiments, the 5′ ligation sequence or the 3′ ligation sequence comprises a nucleic acid sequence having at least 90%, e.g., at least 90%, 91%, 92%, 93%, 94%, 95%, 96%, 97%, 98%, 99% or more sequence identity to SEQ ID NO: 1415. In some instances, the 5′ and 3′ ligation sequences both include the nucleic acid sequence of SEQ ID NO: 1415. The 5′ and 3′ ligation sequences can comprise the same nucleic acid sequence. The 5′ and 3′ ligation sequences can have the same nucleic acid sequence. The 5′ and 3′ ligation sequences can have different nucleic acid sequences. The 5′ and 3′ ligation sequences can comprise different nucleic acid sequences. In some embodiments, the 5′ ligation sequence is SEQ ID NO: 1413 and the 3′ ligation sequence is SEQ ID NO: 1415.

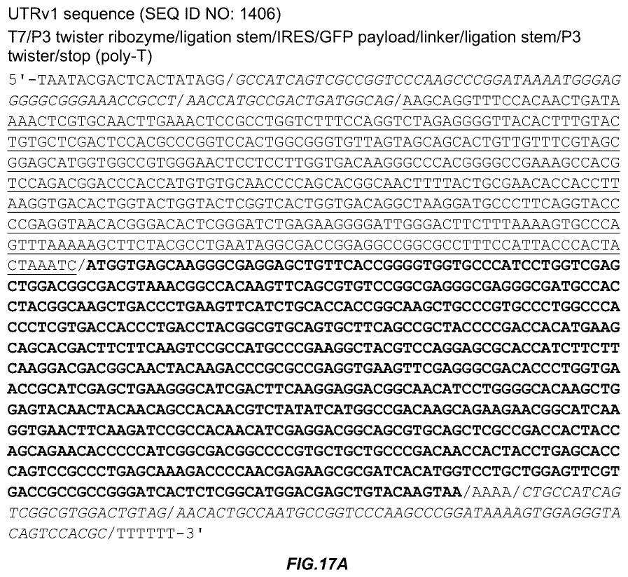

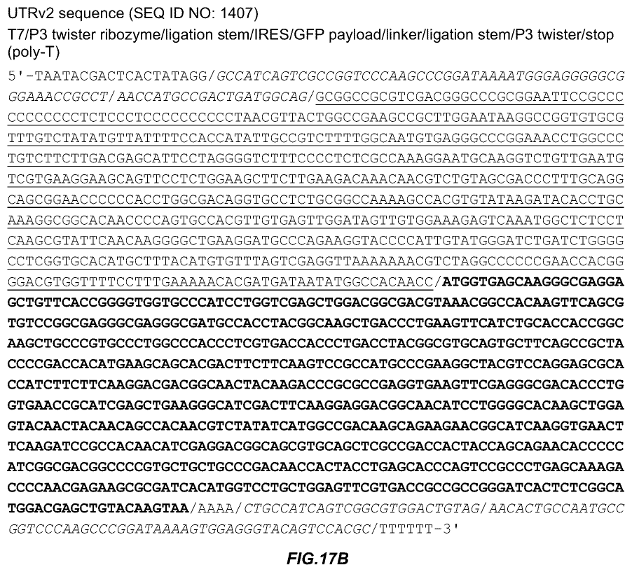

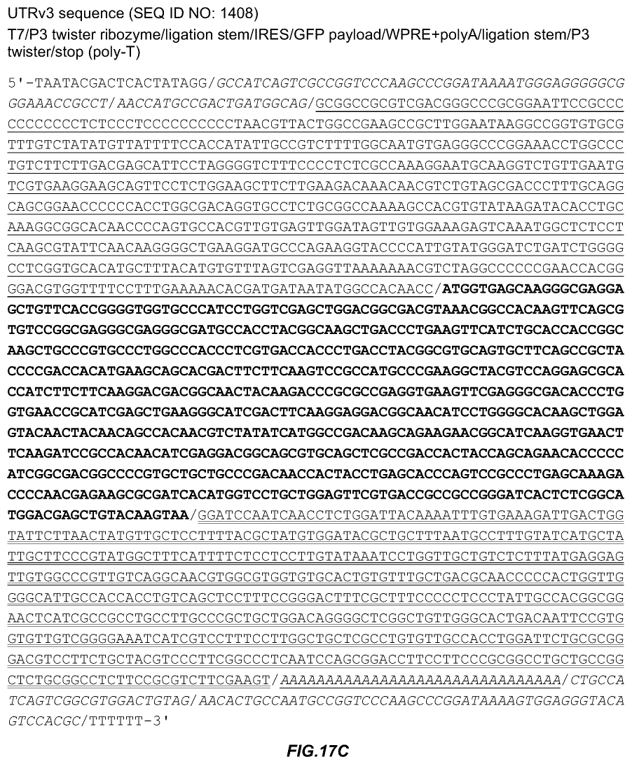

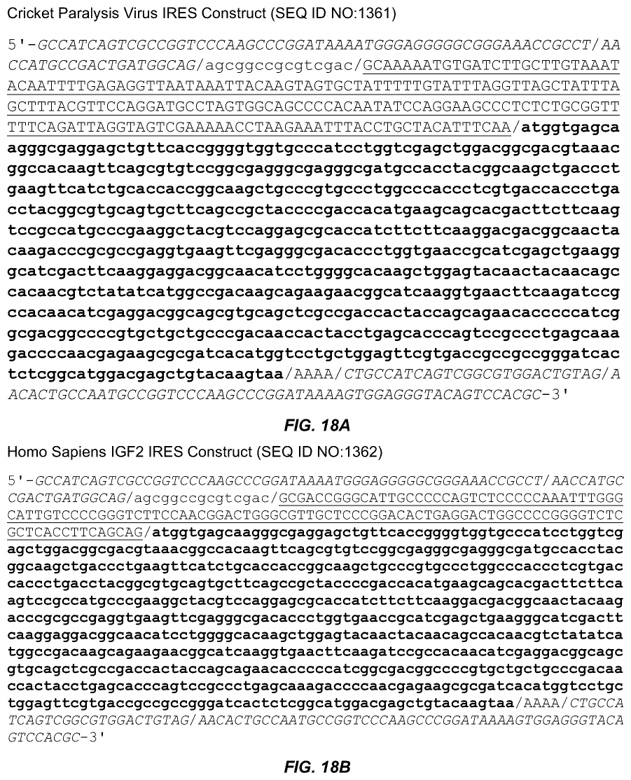

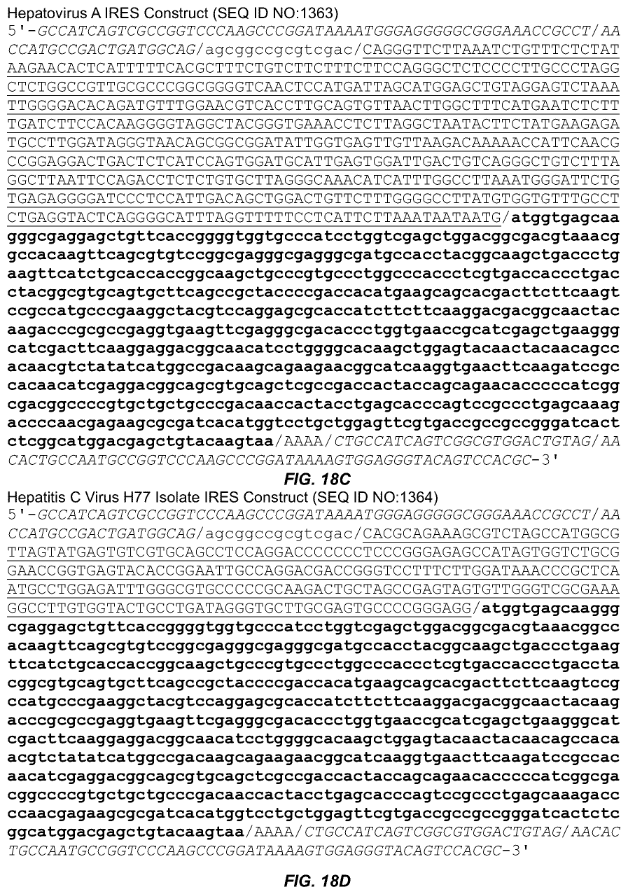

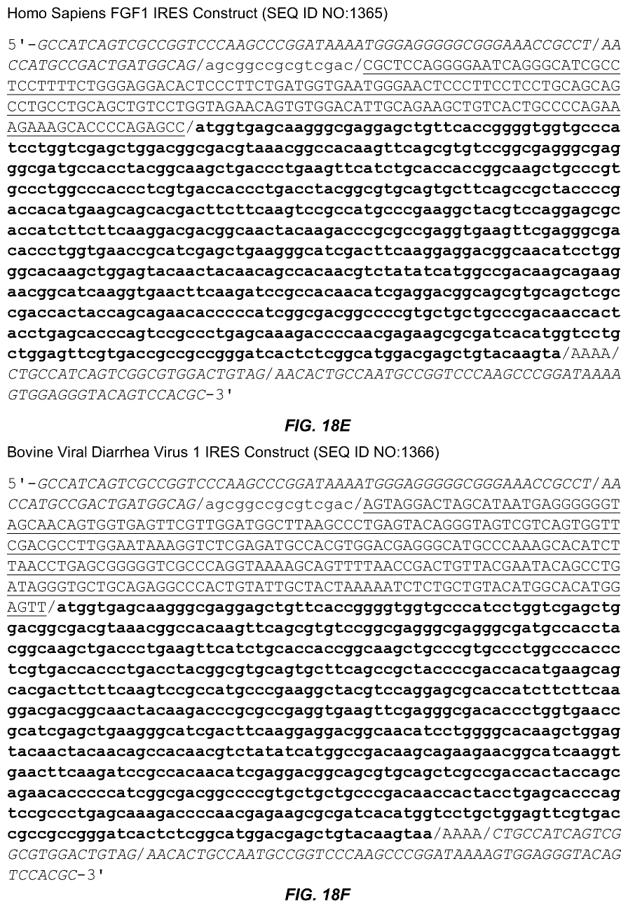

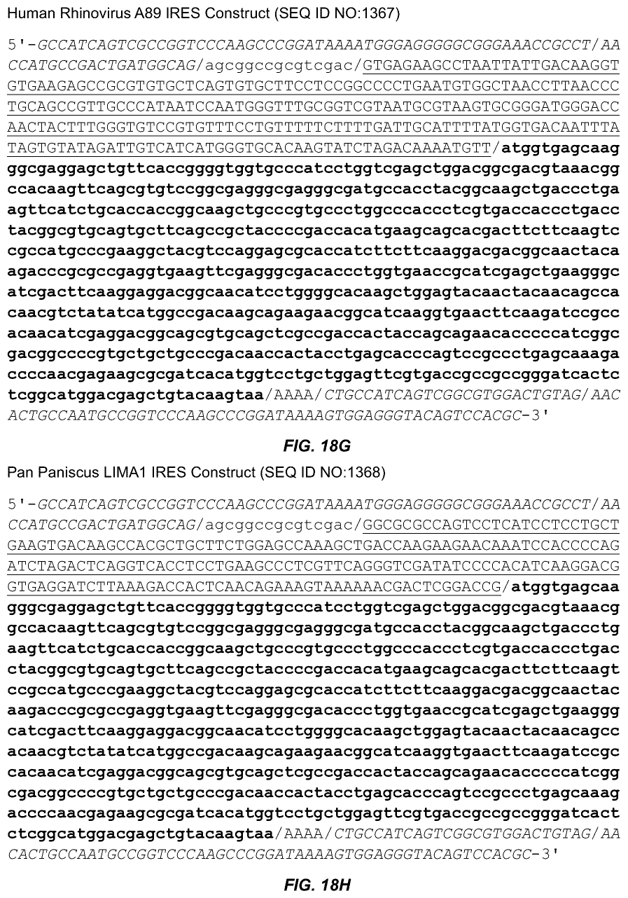

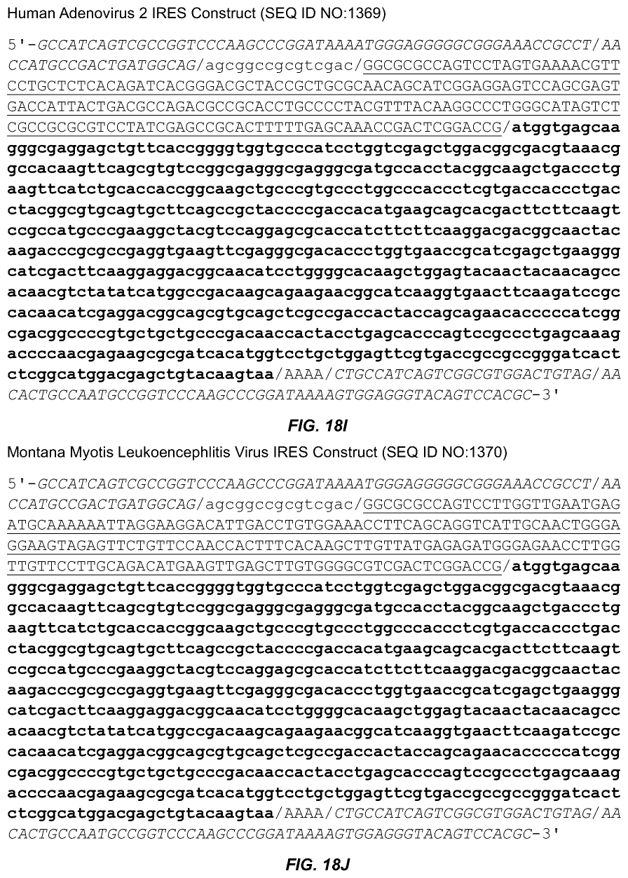

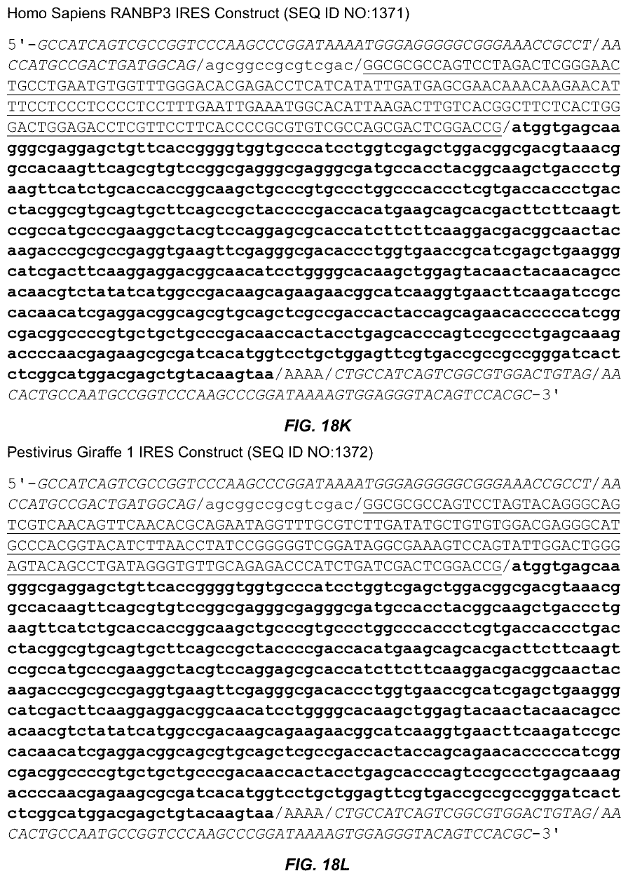

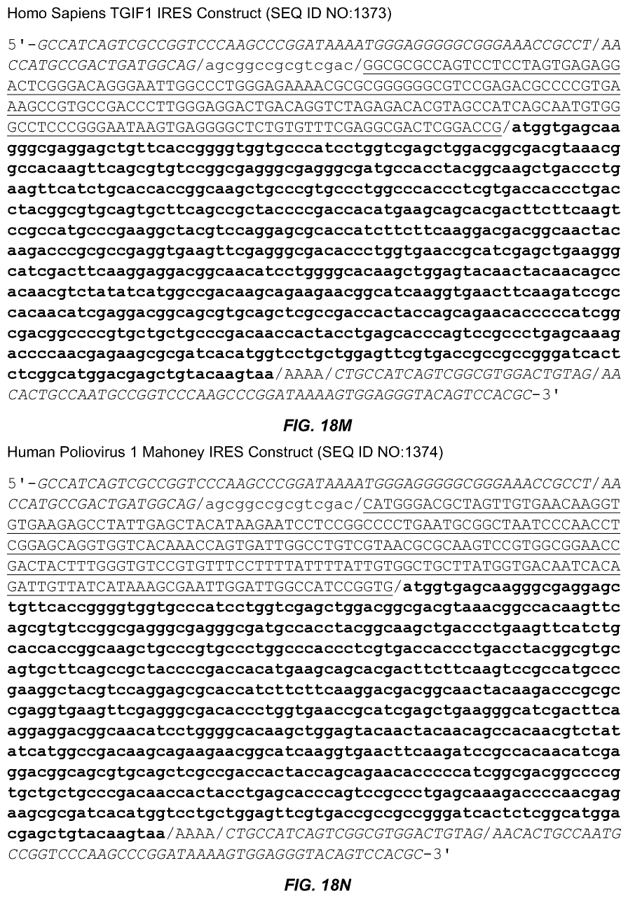

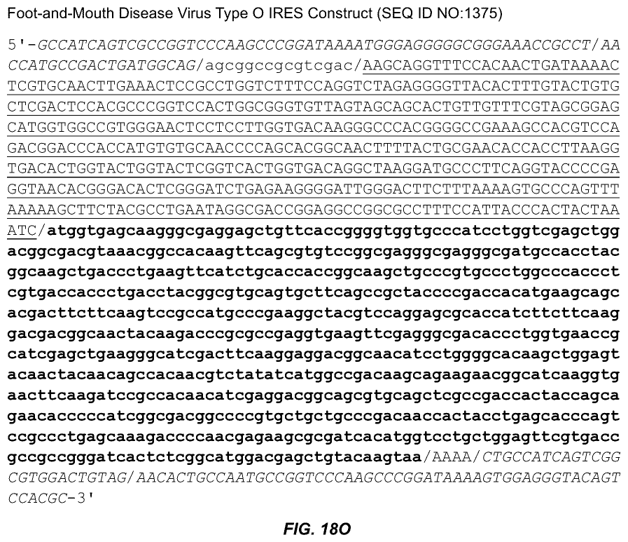

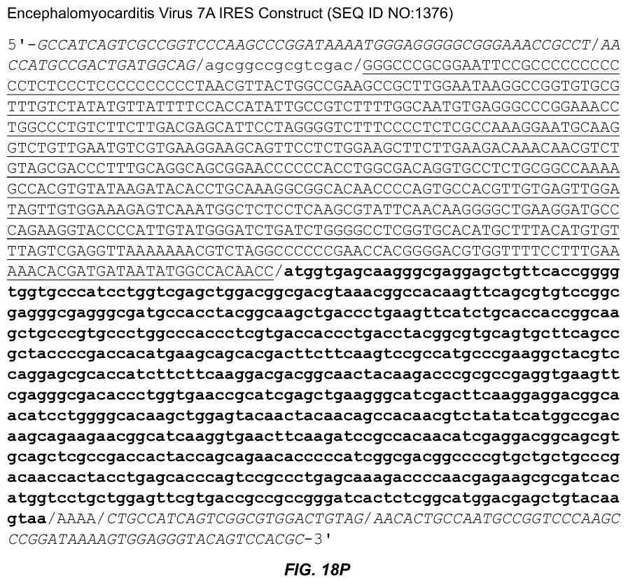

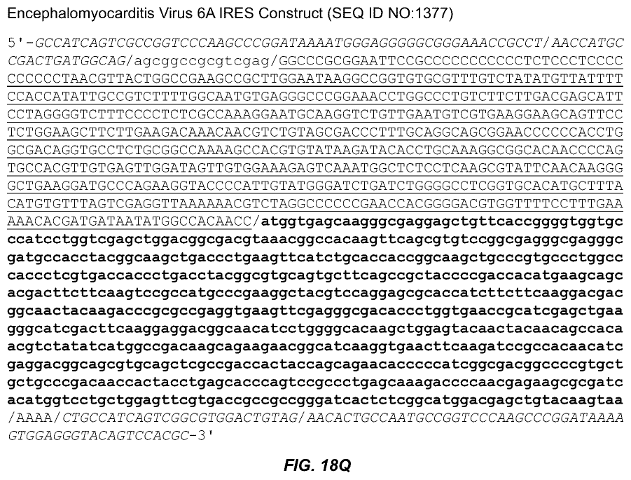

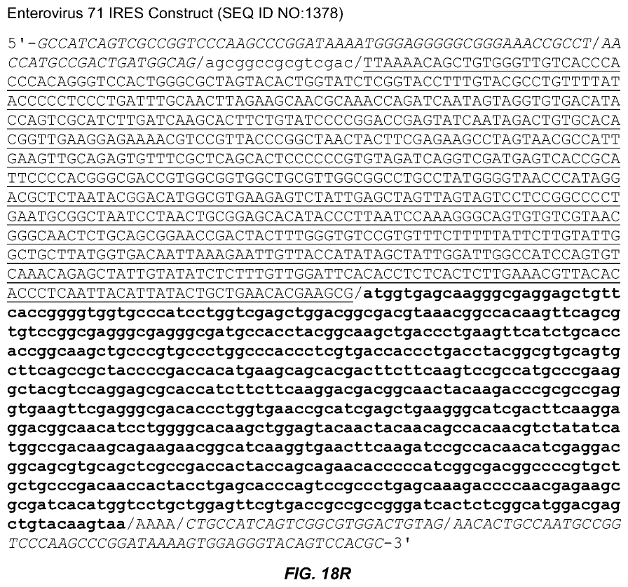

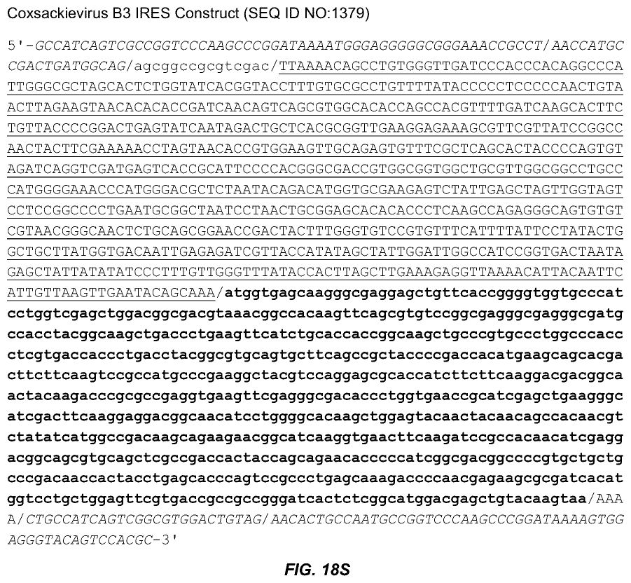

In some embodiments, the IRES is selected from the group consisting of a cricket paralysis virus IRES, a Homo sapiens IGF2 IRES, a hepatovirus A IRES, a hepatitis C virus H77 isolate IRES, a Homo sapiens FGF1 IRES, a bovine viral diarrhea virus 1 IRES, a human rhinovirus A89 IRES, a pan paniscus LIMA1, a human adenovirus 2 IRES, a Montana myotis leukoencephalitis virus IRES, a Homo sapiens RANBP3 IRES, a pestivirus giraffe 1 IRES, a Homo sapiens TGIF1 IRES, a human poliovirus 1 mahoney IRES, a foot-and-mouth disease virus type O IRES, an encephalomyocarditis virus 7A IRES, an encephalomyocarditis virus 6A IRES, an enterovirus 71 IRES, a coxsackievirus B3 IRES, and an IRES sequence provided in the sequence listing including in SEQ ID NOS: 1-1348, and 1361-1391 and the figures such as FIGS. 17-19, where the T nucleotides can be U nucleotides in an RNA polynucleotide. In some embodiments, the IRES sequence is a nucleic acid sequence having at least 90% (e.g., at least 90%, 91%, 92%, 93%, 94%, 95%, 96%, 97%, 98%, 99% or more) sequence identity to any one of SEQ ID NOS: 1-1348, and 1361-1391 and any one shown in FIGS. 17-19, where the T nucleotides can be U nucleotides in an RNA polynucleotide.

In some embodiments, the WPRE sequence comprises the sequence of SEQ ID NO:1353, wherein the T nucleotides can also be U nucleotides in an RNA polynucleotide. In some embodiments, the WPRE comprises a nucleic acid sequence having at least 90% (e.g., at least 90%, 91%, 92%, 93%, 94%, 95%, 96%, 97%, 98%, 99% or more) sequence identity to of SEQ ID NO:1353, where the T nucleotides can be U nucleotides in an RNA polynucleotide.

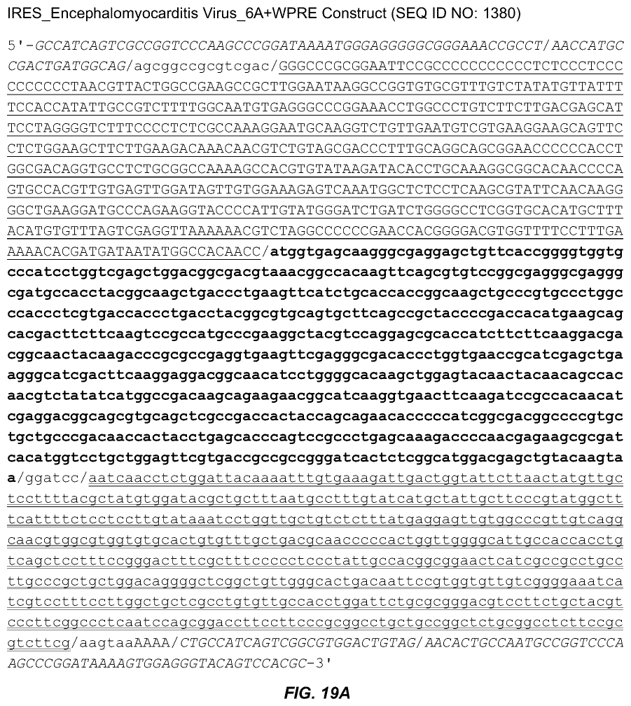

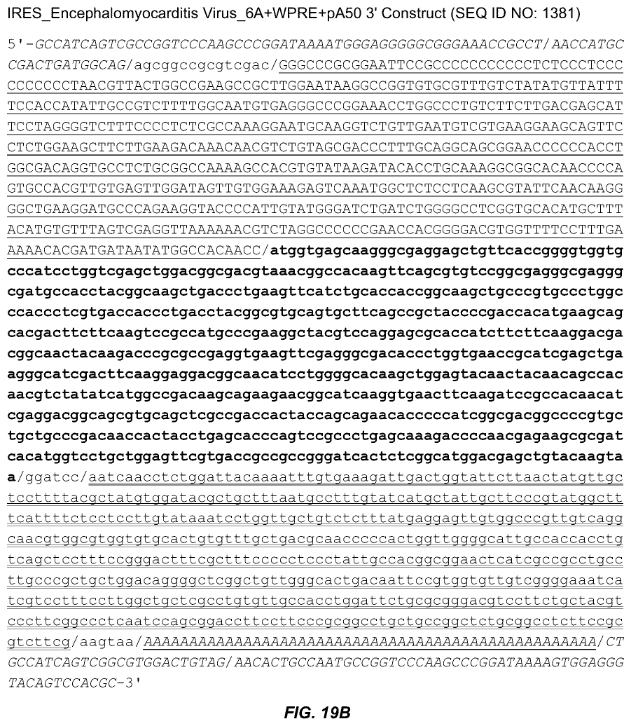

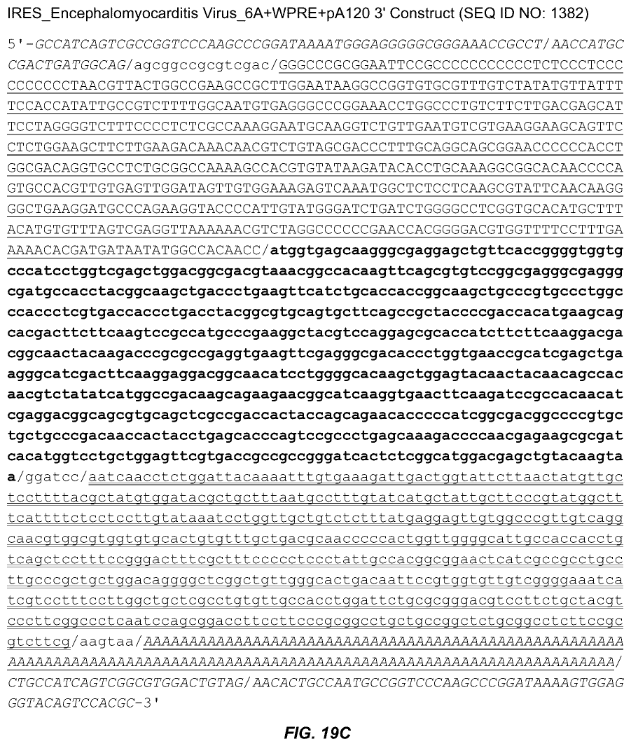

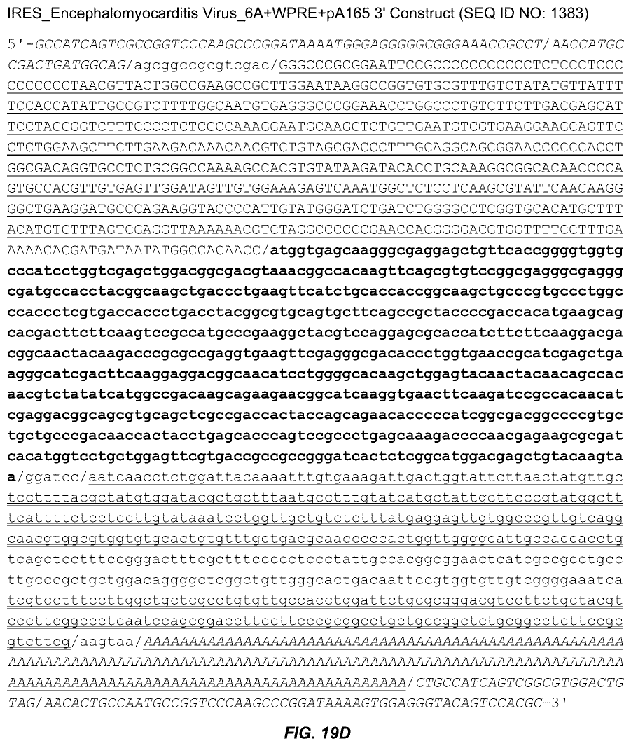

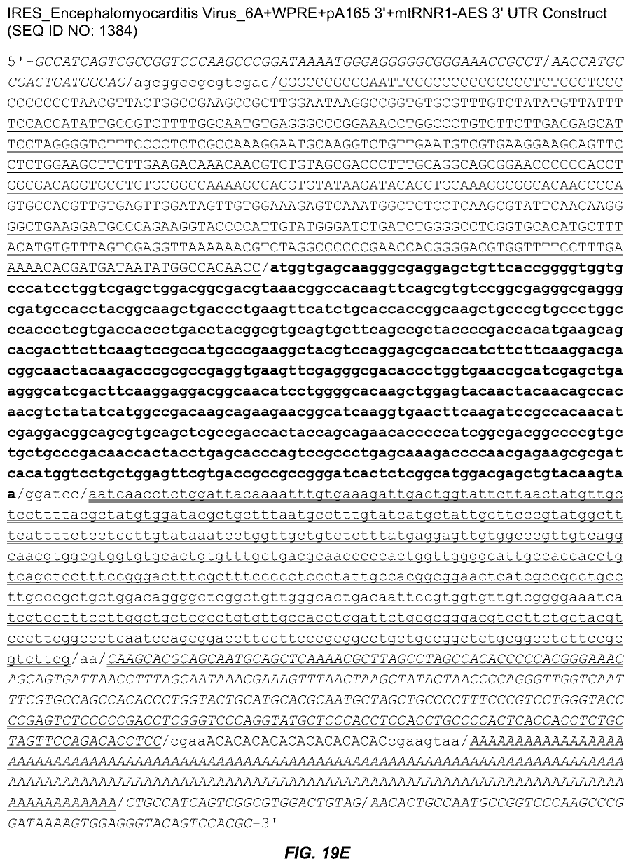

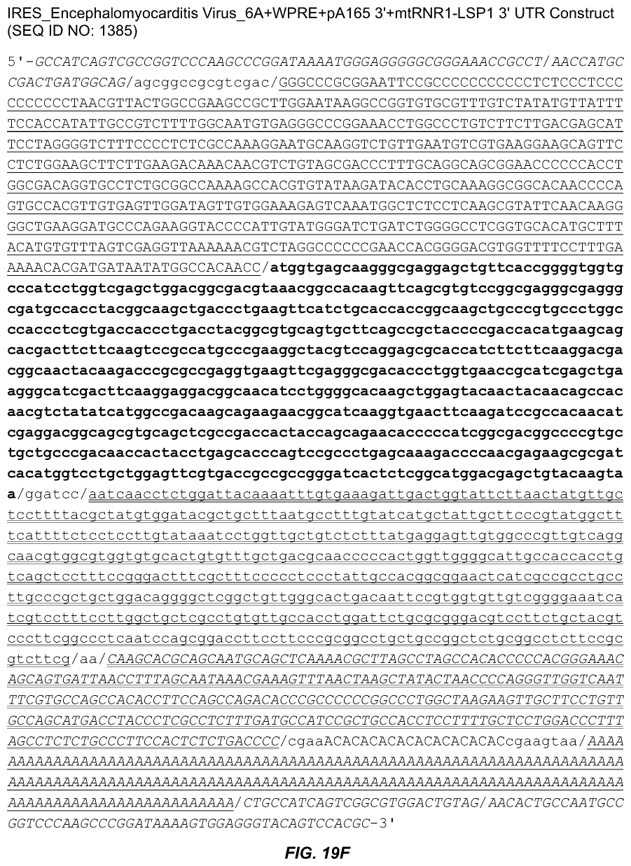

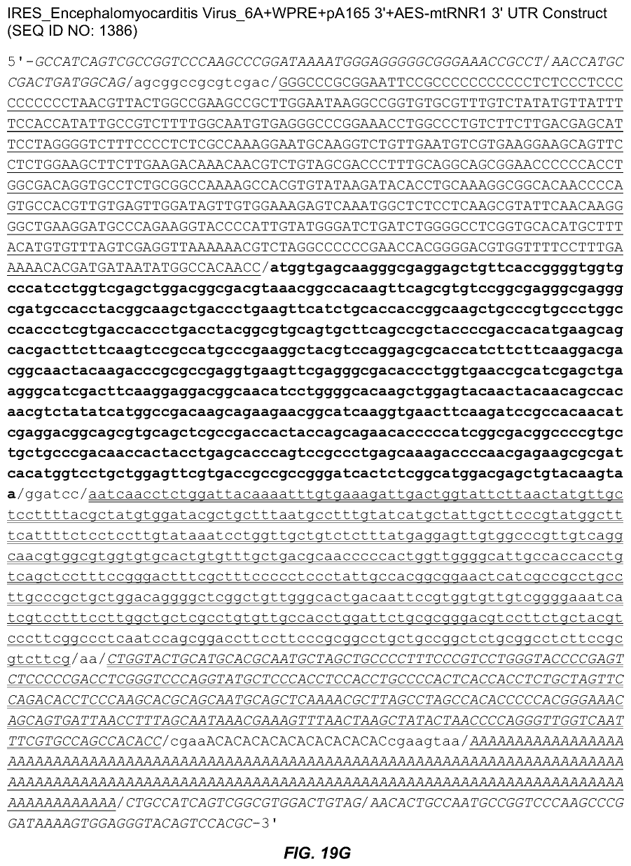

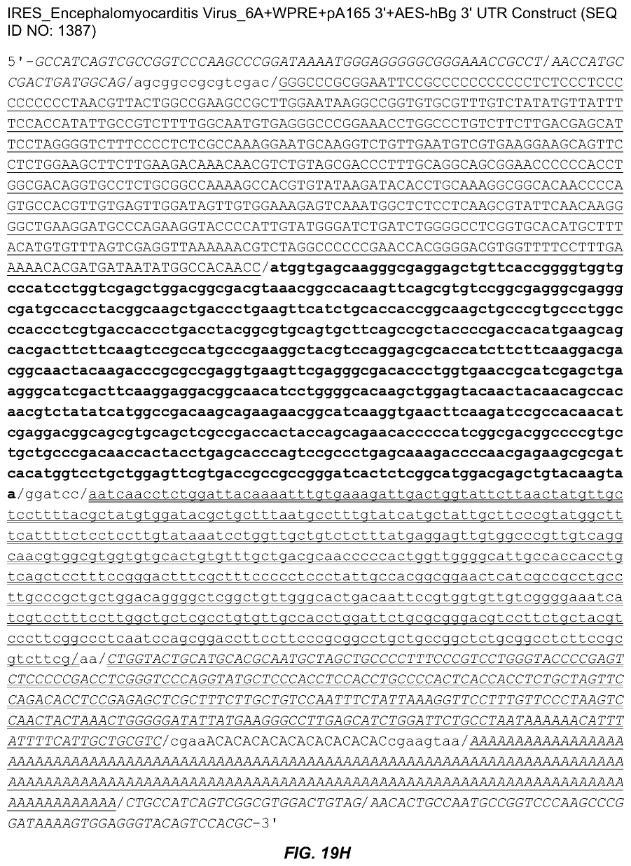

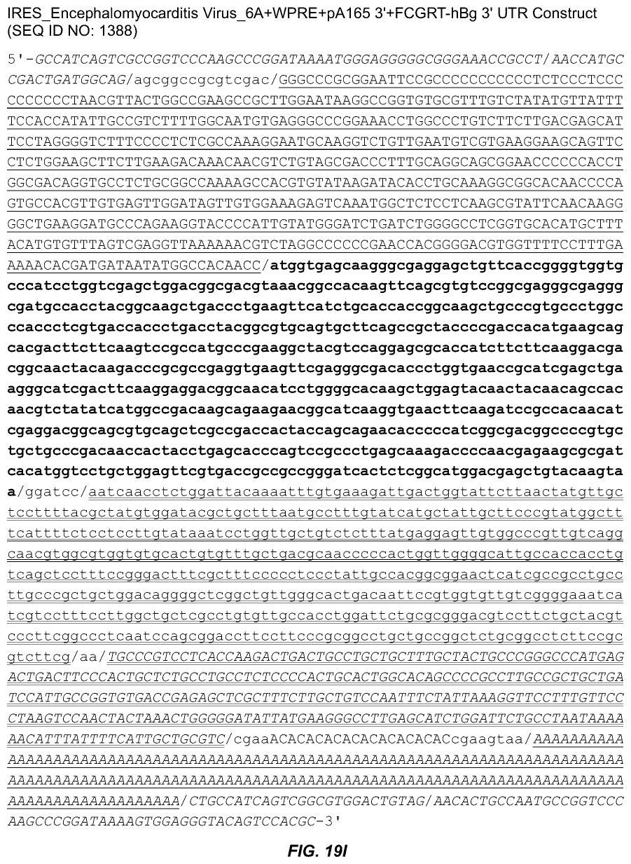

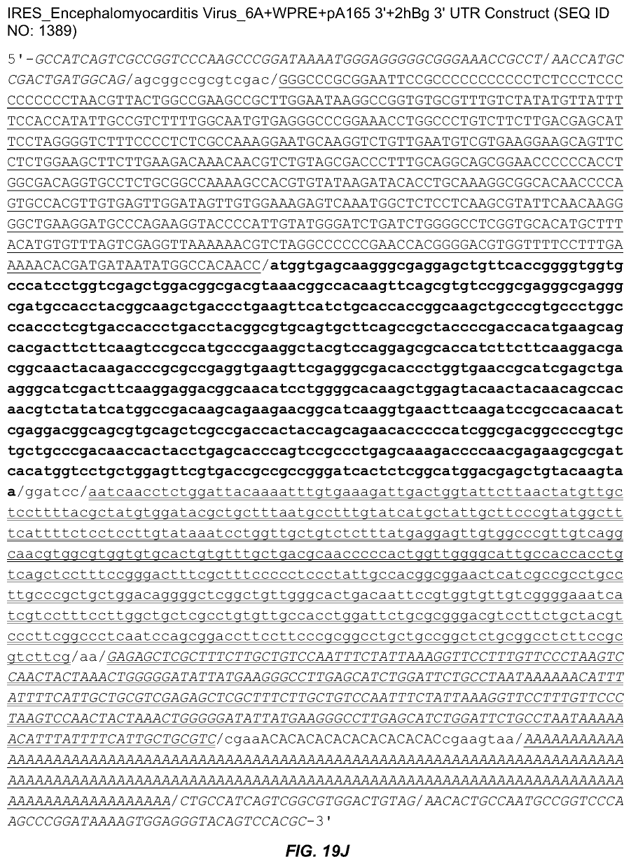

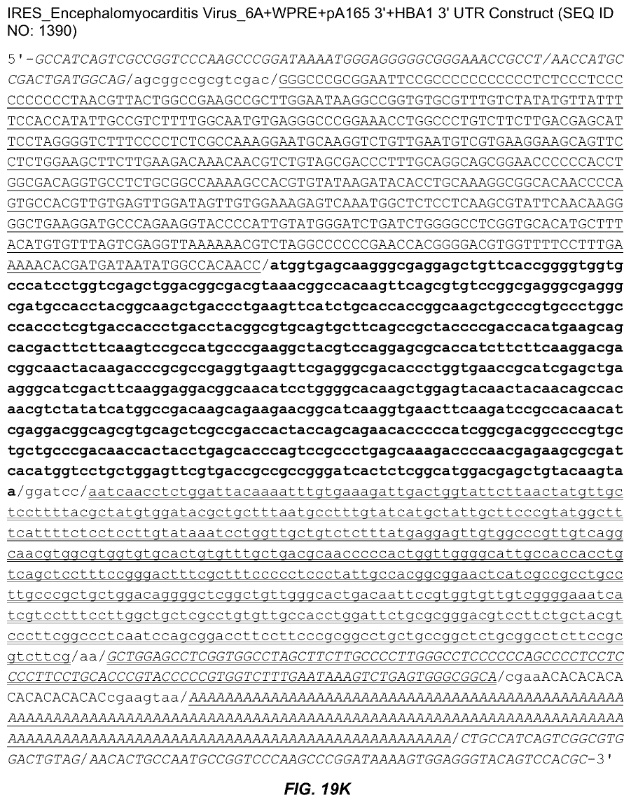

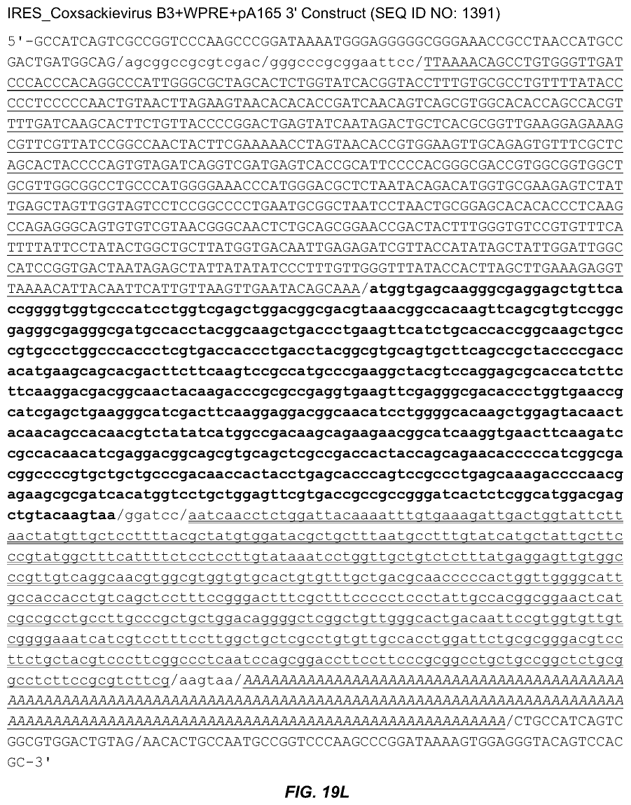

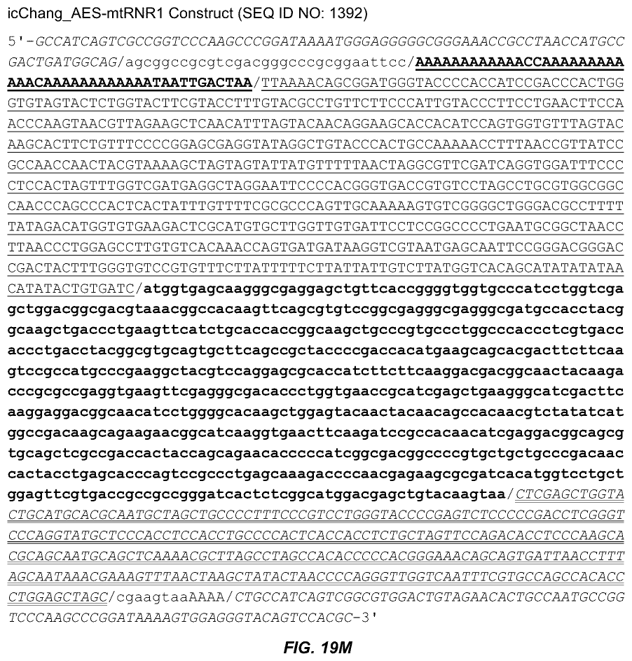

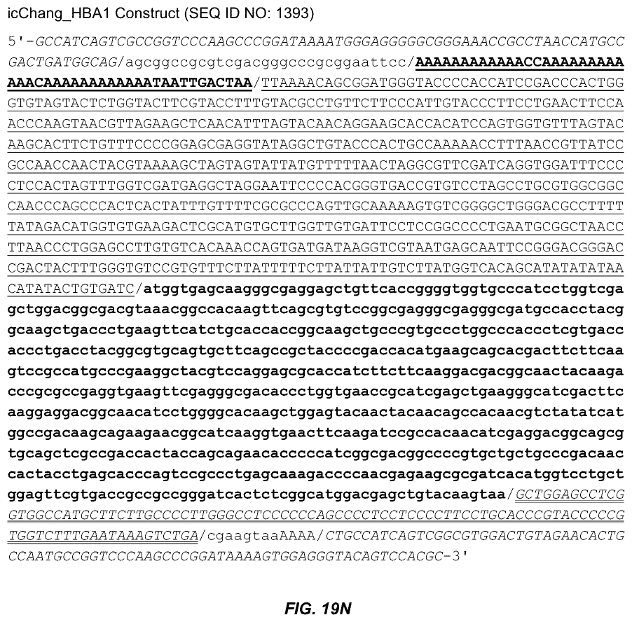

In some embodiments, the 3′UTR sequence is selected from the group consisting of an mtRNR1-AES 3′ UTR, an mtRNR1-LSP1 3′ UTR, an AES-mtRNR1 3′ UTR, an AES-hBg 3′ UTR, an FCGRT-hBg 3′ UTR, a 2hBg 3′ UTR, and a HBA1 3′ UTR. A 3′ UTR can comprise a sequence selected from the group consisting of SEQ ID NOS: 1354-1360 and 1384-1390 and the figures such as FIG. 19, where the T nucleotides can be U nucleotides in a RNA polynucleotide. In some embodiments, the 3′UTR sequence is a nucleic acid sequence having at least 90% (e.g., at least 90%, 91%, 92%, 93%, 94%, 95%, 96%, 97%, 98%, 99% or more) sequence identity to any one of SEQ ID NOS: 1354-1360 and 1384-1390 and any one shown in FIG. 19, where the T nucleotides can be U nucleotides in a RNA polynucleotide.

In some embodiments, the poly(A) sequence is a stretch or chain of about 5-500, 10-500, 20-500, 50-500, 70-500, 80-500, 90-500, 100-500, 110-500, 120-500, 130-500, 140-500, 150-500, 160-500, 165-500, 170-500, 180-500, 190-500, 200-500, 10-200, 20-200, 30-200, 40-200, 50-200, 60-200, 70-200, 80-200, 90-200, 100-200, 50, 120, 150, 165, 200, 500, or more (or any value between a preceding range of values) adenine nucleotides. In some embodiments, the poly(A) sequence is a stretch or chain of 5-500, 10-500, 20-500, 50-500, 70-500, 80-500, 90-500, 100-500, 110-500, 120-500, 130-500, 140-500, 150-500, 160-500, 165-500, 170-500, 180-500, 190-500, 200-500, 10-200, 20-200, 30-200, 40-200, 50-200, 60-200, 70-200, 80-200, 90-200, 100-200, 50, 120, 150, 165, 200, 500, or more (or any value between a range of preceding values) adenine nucleotides.

In some embodiments, the linearized ribozyme activated RNA construct comprises an IRES sequence, a WPRE sequence, and a poly(A) sequence of any depicted in SEQ ID NOs: 1381-1391 and FIG. 19 (please note that the sequences in FIG. 19 include the eGFP coding sequence (bold) as the payload sequence or polypeptide of interest sequence). In some embodiments, the linearized ribozyme activated RNA construct comprises the IRES sequence, the WPRE sequence, a 3′ UTR sequence, and the poly(A) sequence of any depicted in SEQ ID NOs: 1384-1391 and FIG. 19, where the T nucleotides are U nucleotides in the RNA polynucleotide.

In some embodiments, the linearized ribozyme activated RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) a poly(A) sequence (e.g., 10-180 adenine nucleotides), and (vi) a 3′ ligation sequence of SEQ ID NO: 1415, where the T nucleotides are U nucleotides in the RNA polynucleotide.

In some embodiments, the linearized ribozyme activated RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) any 3′ UTR sequence provided herein, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In some embodiments, the linearized ribozyme activated RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) mtRNR1-AES 3′ UTR of SEQ ID NO: 1354, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In some embodiments, the linearized ribozyme activated RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) mtRNR1-LSP1 3′ UTR of SEQ ID NO: 1355, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In particular embodiments, the linearized ribozyme activated RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) AES-mtRNR1 3′ UTR of SEQ ID NO: 1356, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In particular embodiments, the linearized ribozyme activated RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) AES-hBg 3′ UTR of SEQ ID NO: 1357, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In various embodiments, the linearized ribozyme activated RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) FCGRT-hBg 3′ UTR of SEQ ID NO: 1358, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In many embodiments, the linearized ribozyme activated RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) 2hBG 3′ UTR of SEQ ID NO: 1359, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In some embodiments, the linearized ribozyme activated RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) HBA1 3′ UTR of SEQ ID NO: 1360, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In other embodiments, the linearized ribozyme activated RNA construct comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iii) a WPRE sequence of SEQ ID NO: 1353, (iv) a poly(A) sequence (e.g., 10-180 adenine nucleotides), and (v) a 3′ ligation sequence of SEQ ID NO: 1415, where the T nucleotides are U nucleotides in the RNA polynucleotide.

In some embodiments, the linearized ribozyme activated RNA construct comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) any 3′ UTR sequence provided herein, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In some embodiments, the linearized ribozyme activated RNA construct comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) mtRNR1-AES 3′ UTR of SEQ ID NO: 1354, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In some embodiments, the linearized ribozyme activated RNA construct comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) mtRNR1-LSP1 3′ UTR of SEQ ID NO: 1355, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In particular embodiments, the linearized ribozyme activated RNA construct comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) AES-mtRNR1 3′ UTR of SEQ ID NO: 1356, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In particular embodiments, the linearized ribozyme activated RNA construct comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) AES-hBg 3′ UTR of SEQ ID NO: 1357, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In various embodiments, the linearized ribozyme activated RNA construct comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) FCGRT-hBg 3′ UTR of SEQ ID NO: 1358, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In many embodiments, the linearized ribozyme activated RNA construct comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) 2hBG 3′ UTR of SEQ ID NO: 1359, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

In some embodiments, the linearized ribozyme activated RNA construct comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) HBA1 3′ UTR of SEQ ID NO: 1360, (vi) a poly(A) sequence (e.g., 10-180 adenine nucleotides), and (vii) a 3′ ligation sequence of SEQ ID NO: 1415, where the T nucleotides are U nucleotides in the RNA polynucleotide.

The linearized ribozyme activated RNA construct can form a circular RNA polynucleotide. In other words, a linear icRNA can circularize to form a circular icRNA. In some embodiments, the circular RNA polynucleotide is formed in the presence of a linearized ribozyme activated RNA construct such as a linear ribozyme activated RNA construct that lacks ribozymes, such as twister ribozymes. In some embodiments, the circular RNA polynucleotide is formed in a cell and in the presence of a linear RNA polynucleotide such as a linear ribozyme activated RNA construct that lacks ribozymes, such as twister ribozymes. In some embodiments, the circular RNA polynucleotide is formed in the absence of a corresponding DNA construct encoding the linearized ribozyme activated RNA construct. In some embodiments, the circular RNA polynucleotide is formed in the absence of a corresponding DNA construct encoding the linearized ribozyme activated RNA construct in a cell. In some embodiments, the circular RNA polynucleotide is formed in cell lacking a corresponding DNA construct encoding the linearized ribozyme activated RNA construct. In some embodiments, the circular RNA polynucleotide is formed in the absence of a linear ribozyme-RNA polynucleotide such as a corresponding RNA construct that further comprises one or more ribozymes (e.g., twister ribozymes) encoding the linearized ribozyme activated RNA construct. In some embodiments, the circular RNA polynucleotide is formed in the absence of a linear ribozyme-RNA polynucleotide such as a corresponding RNA construct that further comprises one or more ribozymes (e.g., twister ribozymes) encoding the linearized ribozyme activated RNA construct in a cell. In some embodiments, the circular RNA polynucleotide is formed in cell lacking a linear ribozyme-RNA polynucleotide such as a corresponding RNA construct that further comprises one or more ribozymes (e.g., twister ribozymes) encoding the linearized ribozyme activated RNA construct. A linear ribozyme-RNA construct can be formed from transcription of a corresponding DNA construct encoding the linear ribozyme-RNA construct. In some embodiments, the circular RNA polynucleotide is formed in the presence of an RtcB protein. In some embodiments, the linearized ribozyme activated RNA construct is contacted or incubated with an RtcB protein in vitro or in vivo. In some embodiments, the linearized ribozyme activated RNA construct is contacted or incubated with an RtcB protein in a cell. In some embodiments, the linearized ribozyme activated RNA construct is circularized in a cell such as a cell expressing an RtcB protein. The RtcB protein in the cell can be an endogenous RtcB protein to that cell. The RtcB protein in the cell can be an exogenous RtcB protein that is delivered to that cell or expressed from a nucleic acid construct delivered to that cell. In many embodiments, the linearized ribozyme activated RNA construct is introduced (e.g., delivered or transfected) into a cell or an organism (e.g., a subject including a human subject) by any standard method known to one skilled in the art. In some embodiments, the linearized ribozyme activated RNA construct is delivered to a cell or organism using a lipid nanoparticle, a liposome, a charged polymer, an uncharged polymer, a nanoparticle, a polymer nanoparticle, a surfactant, a penetrating enhancer (including penetrating peptides), a gene transfer agent, a phospholipid, a micelle, a synthetic vector, a macromolecule, a dendrimer, a biopolymer, a viral particle, or any combination thereof. In some instances, the linearized ribozyme activated RNA construct is formulated with a lipid nanoparticle for delivery into a cell or organism. The linearized ribozyme activated RNA construct can be encapsulated within or associated with a lipid nanoparticle. In some embodiment, the RNA polynucleotide-lipid nanoparticle complex is administered to a cell, organism, or human subject according to standard methods known to those skilled in the art.

Detailed descriptions of lipid nanoparticles for RNA delivery can be found in, for example, Hou et al., Nat Rev Mater, 6, 1078-1094 (10 Aug. 2021). In some embodiments, a lipid nanoparticle comprises one or more components selected from the group consisting of: a cationic lipid, a neutral lipid, a steroid, a polymer conjugated lipid, a polymer, and a biodegradable agent. In some embodiments, the lipid nanoparticle comprises (6Z,9Z,28Z,31Z)-heptatriaconta-6,9,28,31-tetraen-19-yl-4-(dimethylamino) butanoate (DLin-MC3-DMA); cholesterol; 1,2-distearoyl-sn-glycero-3-phosphocholine (DSPC); and 1,2-dimyristoyl-rac-glycero-3-methoxypolyethylene glycol-2000 (DMG-PEG-2000). In some embodiments, the lipid nanoparticle has a mole ratio of DLin-MC3-DMA: cholesterol: DSPC: DMG-PEG-2000 is 50:38.5:10:1.5. In some instances, the mole ratio is different and optimized for the target cell or organism. In certain embodiments, the lipid nanoparticle has an N/P ratio ranging from 1-10. In some embodiments, the lipid nanoparticle has an N/P ratio of 5.4. In some instances, the N/P ratio is different and optimized for the target cell or organism.

A linearized ribozyme activated RNA construct described herein comprising a ligation sequence comprising a 5′-OH end and a ligation sequence comprises a 2′, 3′-cyclic phosphate end can be circularized in situ such as in a cell to generate a circular RNA polynucleotide (also referred to herein as circular icRNAs). As such, the resulting in situ circularized RNA (circular icRNA) includes the components of the linearized ribozyme activated RNA construct. In some embodiments, the circular RNA polynucleotide comprises one or more components including an IRES, WPRE, 3′ UTR, poly(A) stretch, or any combination thereof. These one or more components including an IRES, WPRE, 3′ UTR, poly(A) stretch, or any combination thereof of can increase protein translation, protein translation efficiency, and/or protein yield of any polypeptide of interest encoded in the circular RNA polynucleotide as compared to a circular RNA polynucleotide lacking such component(s). In some instances, cells comprising a circular RNA polynucleotide described herein produce a higher level of a polypeptide of interest encoded on the circular RNA as compared to a circular RNA that does not include an IRES, a WPRE, a 3′ UTR, a poly(A) stretch, or a combination thereof.

In some embodiments, the circular RNA polynucleotide comprises one or more components selected from the group consisting of: an IRES, a WPRE, a 3′ UTR sequence, a poly(A), and a polynucleotide of interest. In some embodiments, a spacer or linker sequence is present between any components of the circular RNA polynucleotide. In some embodiments, the polynucleotide of interest encodes a polypeptide of interest such as, but not limited to, a fill-length protein, a fusion protein, a chimeric protein, a recombinant protein, a therapeutic protein, a protein fragment, a truncated protein, and the like. In some embodiments, the circular RNA polynucleotide comprises in 5′ to 3′ order: a ligated sequence, an IRES sequence, a polynucleotide sequence of interest, a WPRE sequence, and a poly(A) sequence, where the ligated sequence is formed from the ligation of the 5′ and 3′ ligation sequences of the linear RNA polynucleotide. See, FIG. 7G of a linear RNA polynucleotide and its ligation sequences and a Sanger sequencing trace corresponding to a resulting circular RNA with the ligation junction. In some embodiments, the circular RNA polynucleotide comprises in 5′ to 3′ order: a ligated sequence, an IRES sequence, a polynucleotide sequence of interest, a WPRE sequence, a 3′ UTR sequence, a poly(A) sequence, where the ligated sequence is formed from the ligation of the 5′ and 3′ ligation sequences of the linear RNA polynucleotide. In some embodiments, the circular RNA polynucleotide comprises in 5′ to 3′ order: an IRES sequence, a polynucleotide sequence of interest, a WPRE sequence, an optional 3′ UTR sequence, and a poly(A) sequence.

In some embodiments, the IRES of the circular RNA polynucleotide is any selected from the group consisting of a cricket paralysis virus IRES, a Homo sapiens IGF2 IRES, a hepatovirus A IRES, a hepatitis C virus H77 isolate IRES, a Homo sapiens FGF1 IRES, a bovine viral diarrhea virus 1 IRES, a human rhinovirus A89 IRES, a pan paniscus LIMA1, a human adenovirus 2 IRES, a Montana myotis leukoencephalitis virus IRES, a Homo sapiens RANBP3 IRES, a pestivirus giraffe 1 IRES, a Homo sapiens TGIF1 IRES, a human poliovirus 1 mahoney IRES, a foot-and-mouth disease virus type O IRES, an encephalomyocarditis virus 7A IRES, an encephalomyocarditis virus 6A IRES, an enterovirus 71 IRES, a coxsackievirus B3 IRES, and an IRES sequence provided in the sequence listing including in SEQ ID NOS: 1-1348, and 1361-1391 and the figures such as FIGS. 17-19, where the T nucleotides are U nucleotides in a RNA polynucleotide. In some embodiments, the IRES sequence is a nucleic acid sequence having at least 90%, e.g., at least 90%, 91%, 92%, 93%, 94%, 95%, 96%, 97%, 98%, 99% or more, sequence identity to any one of SEQ ID NOS: 1-1348, and 1361-1391 and any one shown in FIGS. 17-19, where the T nucleotides are U nucleotides in a RNA polynucleotide. In some embodiments, the WPRE sequence of the circular RNA polynucleotide is the nucleic acid sequence of SEQ ID NO:1353, wherein the T nucleotides are U nucleotides in an RNA polynucleotide. In some embodiments, the WPRE sequence is a nucleic acid sequence having at least 90%, e.g., at least 90%, 91%, 92%, 93%, 94%, 95%, 96%, 97%, 98%, 99% or more sequence identity to of SEQ ID NO:1353, where the T nucleotides are U nucleotides in an RNA polynucleotide. In some embodiments, the 3′UTR sequence of the circular RNA polynucleotide is any selected from the group consisting of an mtRNR1-AES 3′ UTR, an mtRNR1-LSP1 3′ UTR, an AES-mtRNR1 3′ UTR, an AES-hBg 3′ UTR, an FCGRT-hBg 3′ UTR, a 2hBg 3′ UTR, and a HBA1 3′ UTR, a 3′UTR sequence provided in the sequence listing including in SEQ ID NOS: 1354-1360 and 1384-1390 and the figures such as FIG. 19, where the T nucleotides are U nucleotides in a RNA polynucleotide. In some embodiments, the 3′UTR sequence is a nucleic acid sequence having at least 90%, e.g., at least 90%, 91%, 92%, 93%, 94%, 95%, 96%, 97%, 98%, 99% or more, sequence identity to any one of SEQ ID NOS: 1354-1360 and 1384-1390 and any one shown in FIG. 19, where the T nucleotides are U nucleotides in a RNA polynucleotide. In some embodiments, the poly(A) sequence of the circular RNA polynucleotide is a stretch or chain of about 5-500, 10-500, 20-500, 50-500, 70-500, 80-500, 90-500, 100-500, 110-500, 120-500, 130-500, 140-500, 150-500, 160-500, 165-500, 170-500, 180-500, 190-500, 200-500, 10-200, 20-200, 30-200, 40-200, 50-200, 60-200, 70-200, 80-200, 90-200, 100-200, 50, 120, 150, 165, 200, 500, or more adenine nucleotides.

In some embodiments, the circular RNA polynucleotide comprises the IRES sequence, the WPRE sequence, and the poly(A) sequence of any depicted in SEQ ID NOs: 1381-1391 and FIG. 19. In some embodiments, the circular RNA polynucleotide comprises the IRES sequence, the WPRE sequence, a 3′ UTR sequence, and the poly(A) sequence of any depicted in SEQ ID NOs: 1384-1391 and FIG. 19, where the T nucleotides are U nucleotides in the RNA polynucleotide. In some embodiments, the circular RNA polynucleotide comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) a poly(A) sequence (e.g., 10-180 adenine nucleotides), and (vi) a 3′ ligation sequence of SEQ ID NO: 1415, where the ligation sequences are ligated together and the T nucleotides are U nucleotides in the circular RNA polynucleotide.

In some embodiments, the circular RNA polynucleotide comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) any 3′ UTR sequence provided herein, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In some embodiments, the circular RNA polynucleotide comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) mtRNR1-AES 3′ UTR of SEQ ID NO: 1354, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In some embodiments, the circular RNA polynucleotide comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) mtRNR1-LSP1 3′ UTR of SEQ ID NO: 1355, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In particular embodiments, the circular RNA polynucleotide comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) AES-mtRNR1 3′ UTR of SEQ ID NO: 1356, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In particular embodiments, the circular RNA polynucleotide comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) AES-hBg 3′ UTR of SEQ ID NO: 1357, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In various embodiments, the circular RNA polynucleotide comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) FCGRT-hBg 3′ UTR of SEQ ID NO: 1358, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In many embodiments, the circular RNA polynucleotide comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) 2hBG 3′ UTR of SEQ ID NO: 1359, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In some embodiments, the circular RNA polynucleotide includes in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) HBA1 3′ UTR of SEQ ID NO: 1360, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide.

In other embodiments, the circular RNA polynucleotide comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iii) a WPRE sequence of SEQ ID NO: 1353, (iv) a poly(A) sequence (e.g., 10-180 adenine nucleotides), and (v) a 3′ ligation sequence of SEQ ID NO: 1415, where the ligation sequences are ligated together and the T nucleotides are U nucleotides in the circular RNA polynucleotide. In some embodiments, the circular RNA polynucleotide comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) any 3′ UTR sequence provided herein, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In some embodiments, the circular RNA polynucleotide comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) mtRNR1-AES 3′ UTR of SEQ ID NO: 1354, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In some embodiments, the circular RNA polynucleotide comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) mtRNR1-LSP1 3′ UTR of SEQ ID NO: 1355, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In particular embodiments, the circular RNA polynucleotide comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) AES-mtRNR1 3′ UTR of SEQ ID NO: 1356, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In particular embodiments, the circular RNA polynucleotide comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) AES-hBg 3′ UTR of SEQ ID NO: 1357, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In various embodiments, the circular RNA polynucleotide comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) FCGRT-hBg 3′ UTR of SEQ ID NO: 1358, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In many embodiments, the circular RNA polynucleotide comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) 2hBG 3′ UTR of SEQ ID NO: 1359, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide. In some embodiments, the circular RNA polynucleotide comprises in 5′ to 3′ order: (i) a 5′ ligation sequence of SEQ ID NO: 1413, (ii) an IRES sequence of SEQ ID NO: 1348 (a coxsackievirus B3 IRES), (iii) a polynucleotide sequence of interest, (iv) a WPRE sequence of SEQ ID NO: 1353, (v) HBA1 3′ UTR of SEQ ID NO: 1360, (vi) a poly(A) sequence (e.g., 10-180 adenine nucleotides), and (vii) a 3′ ligation sequence of SEQ ID NO: 1415, where the ligation sequences are ligated together and the T nucleotides are U nucleotides in the circular RNA polynucleotide.

As described above, a linearized ribozyme activated RNA construct that is a precursor form of a circular RNA polynucleotide includes two ligation sequences located at opposite ends of the linearized ribozyme activated RNA construct, where one ligation sequence includes a 5′ hydroxyl end and the other ligation sequence includes a 2′,3′-cyclic phosphate end. In some embodiments, the ligation sequences are complementary. In some embodiments, the ligation sequences are partially complementary. In some embodiments, the ligation sequences or a portion thereof hybridize together in a cell and/or in standard in vitro conditions. In some embodiments, the ligation sequences are at least 85%, e.g., at least 85%, 86%, 87%, 88%, 89%, 90%, 91%, 92%, 93%, 94%, 95%, 96%, 97%, 98%, 99% or more complementary over the length of one of the sequences. In some embodiments, the ligation sequences are 100% complementary over the length of at least one of the sequences. In some instances, the 5′ ligation sequence is at least 85%, e.g., at least 85%, 86%, 87%, 88%, 89%, 90%, 91%, 92%, 93%, 94%, 95%, 96%, 97%, 98%, 99% or more complementary to the 3′ ligation sequence. In various instances, the 3′ ligation sequence is at least 85%, e.g., at least 85%, 86%, 87%, 88%, 89%, 90%, 91%, 92%, 93%, 94%, 95%, 96%, 97%, 98%, 99% or more complementary to the 5′ ligation sequence. In some embodiments, the linearized ribozyme activated RNA construct is found in a structure where the 5′ ligation sequence and the 3′ ligation sequence hybridize (see, for example, the linearized ribozyme activated RNA construct without ribozymes in FIGS. 1A, 7A, and 7G). In some embodiments, the 5′ ligation sequence or the 3′ ligation sequence includes the nucleic acid sequence of SEQ ID NO: 1413. In some embodiments, the 5′ ligation sequence or the 3′ ligation sequence includes a nucleic acid sequence having at least 90%, e.g., at least 90%, 91%, 92%, 93%, 94%, 95%, 96%, 97%, 98%, 99% or more sequence identity to SEQ ID NO: 1413. In some cases, the 5′ and 3′ ligation sequences both comprise the nucleic acid sequence of SEQ ID NO: 1413. In several embodiments, the 5′ ligation sequence or the 3′ ligation sequence includes the nucleic acid sequence of SEQ ID NO: 1415. In various embodiments, the 5′ ligation sequence or the 3′ ligation sequence comprises a nucleic acid sequence having at least 90%, e.g., at least 90%, 91%, 92%, 93%, 94%, 95%, 96%, 97%, 98%, 99% or more sequence identity to SEQ ID NO: 1415. In some instances, the 5′ and 3′ ligation sequences both comprise the nucleic acid sequence of SEQ ID NO: 1415. The 5′ and 3′ ligation sequences can comprise the same nucleic acid sequence. The 5′ and 3′ ligation sequences can have the same nucleic acid sequence. The 5′ and 3′ ligation sequences can comprise different nucleic acid sequences. The 5′ and 3′ ligation sequences can have different nucleic acid sequences. In some embodiments, the 5′ ligation sequence is SEQ ID NO: 1413 and the 3′ ligation sequence is SEQ ID NO: 1415. In some instances, a portion of the 5′ ligation sequence is complementary to a portion the 3′ ligation sequence of a linear RNA polynucleotide. See, for example, FIGS. 7F and 7G.

icRNAs including icRNAs in a linear or circular RNA format can be produced from a DNA construct encoding the icRNAs. In some embodiments, the DNA construct (such as a linear or circular DNA construct) comprises a nucleic acid sequence encoding any of the linear RNA polynucleotides described herein. In some embodiments, the DNA construct (such as a linear or circular DNA construct) comprises a nucleic acid sequence encoding any of the circular RNA polynucleotides described herein. In some embodiments, the DNA construct (such as a linear or circular DNA construct) comprises a nucleic acid sequence encoding any of the linear RNA polynucleotides described herein, include a linear RNA polynucleotide comprising a ribozyme (also referred to as a linearized ribozyme-RNA construct) or a linear RNA polynucleotide lacking a ribozyme (also referred to as linearized ribozyme activated RNA construct or linear icRNA).

In some embodiments, a DNA construct comprises in 5′ to 3′ order: a promoter (e.g., a T7 promoter), a 5′ ribozyme sequence, a 5′ ligation sequence, an IRES, a polynucleotide sequence of interest, a WPRE, a poly(A), a 3′ ligation sequence, and a 3′ ribozyme sequence. In some embodiments, a spacer or linker sequence is present between any components of the DNA construct.

In various embodiments, the DNA construct comprises in 5′ to 3′ order: a ribozyme (e.g., 5′ ribozyme), a ligation sequence (e.g., 5′ ligation sequence), an IRES sequence, a polynucleotide sequence of interest, a WPRE sequence, a poly(A) sequence, a ligation sequence (e.g., 3′ ligation sequence), and a ribozyme (e.g., 3′ ribozyme). In some embodiments, the DNA construct includes in 5′ to 3′ order: a ribozyme (e.g., 5′ ribozyme), a ligation sequence (e.g., 5′ ligation sequence), an IRES sequence, a polynucleotide sequence of interest, a WPRE sequence, a 3′ UTR sequence, a poly(A) sequence, a ligation sequence (e.g., 3′ ligation sequence), and a ribozyme (e.g., 3′ ribozyme).

In some embodiments, a ribozyme of the DNA construct is selected from the group consisting a twister ribozyme, twister sister (TS) ribozyme, a hammerhead ribozyme, a hairpin ribozyme, a hepatitis delta virus (HDV) ribozyme, a Varkud satellite (VS) ribozyme, a glucosamine-6-phosphate (GimS) ribozyme, a pistol ribozyme, and a hatchet ribozyme. In some embodiments, the 5′ ribozyme and the 3′ ribozyme are the same type of ribozymes. In other embodiments, the 5′ ribozyme and the 3′ ribozyme are different types of ribozymes. In some embodiments, the 5′ ribozyme or the 3′ ribozyme is a P1 twister ribozyme, an equivalent, and variant thereof. In some embodiments, the 5′ ribozyme or the 3′ ribozyme is a P3 twister ribozyme, an equivalent, and variant thereof. Both of the 5′ and 3′ ribozymes can be P1 twister ribozymes. In some cases, both of the 5′ and 3′ ribozymes are P1 twister ribozymes. In some instances, the 5′ ribozyme sequence comprises the nucleic acid of SEQ ID NO: 1349. In some instances, the 3′ ribozyme sequence comprises the nucleic acid of SEQ ID NO: 1350. In some instances, the 5′ ligation sequence comprises the nucleic acid of SEQ ID NO: 1351. In some instances, the 3′ ligation sequence comprises the nucleic acid of SEQ ID NO: 1352.

In some embodiments, the IRES of the DNA construct is any selected from the group consisting of a cricket paralysis virus IRES, a Homo sapiens IGF2 IRES, a hepatovirus A IRES, a hepatitis C virus H77 isolate IRES, a Homo sapiens FGF1 IRES, a bovine viral diarrhea virus 1 IRES, a human rhinovirus A89 IRES, a pan paniscus LIMA1, a human adenovirus 2 IRES, a Montana myotis leukoencephalitis virus IRES, a Homo sapiens RANBP3 IRES, a pestivirus giraffe 1 IRES, a Homo sapiens TGIF1 IRES, a human poliovirus 1 mahoney IRES, a foot-and-mouth disease virus type O IRES, an encephalomyocarditis virus 7A IRES, an encephalomyocarditis virus 6A IRES, an enterovirus 71 IRES, a coxsackievirus B3 IRES, and an IRES sequence provided in the sequence listing including in SEQ ID NOS: 1-1329, 1330-1348, and 1361-1391 and the figures such as FIGS. 17-19. In some embodiments, the IRES sequence is a nucleic acid sequence having at least 90%, e.g., at least 90%, 91%, 92%, 93%, 94%, 95%, 96%, 97%, 98%, 99% or more, sequence identity to any one of SEQ ID NOS: 1-1329, 1330-1348, and 1361-1391 and any one shown in FIGS. 17-19. In some embodiments, the WPRE sequence of the DNA construct is the nucleic acid sequence of SEQ ID NO:1353. In some embodiments, the WPRE sequence is a nucleic acid sequence having at least 90%, e.g., at least 90%, 91%, 92%, 93%, 94%, 95%, 96%, 97%, 98%, 99% or more sequence identity to of SEQ ID NO:1353. In some embodiments, the 3′UTR sequence of the DNA construct is any selected from the group consisting of an mtRNR1-AES 3′ UTR, an mtRNR1-LSP1 3′ UTR, an AES-mtRNR1 3′ UTR, an AES-hBg 3′ UTR, an FCGRT-hBg 3′ UTR, a 2hBg 3′ UTR, and a HBA1 3′ UTR, a 3′UTR sequence provided in the sequence listing including in SEQ ID NOS: 1354-1360 and 1384-1390 and the figures such as FIG. 19. In some embodiments, the 3′UTR sequence is a nucleic acid sequence having at least 90%, e.g., at least 90%, 91%, 92%, 93%, 94%, 95%, 96%, 97%, 98%, 99% or more, sequence identity to any one of SEQ ID NOS: 1354-1360 and 1384-1390 and any one shown in FIG. 19. In some embodiments, the poly(A) sequence of DNA construct is a stretch or chain of about 5-500, 10-500, 20-500, 50-500, 70-500, 80-500, 90-500, 100-500, 110-500, 120-500, 130-500, 140-500, 150-500, 160-500, 165-500, 170-500, 180-500, 190-500, 200-500, 10-200, 20-200, 30-200, 40-200, 50-200, 60-200, 70-200, 80-200, 90-200, 100-200, 50, 120, 150, 165, 200, 500, or more adenine nucleotides.

In some embodiments, the DNA construct comprises the IRES sequence, the WPRE sequence, and the poly(A) sequence of any depicted in SEQ ID NOs: 1381-1391 and FIG. 19. In some embodiments, the DNA construct comprises the IRES sequence, the WPRE sequence, a 3′ UTR sequence, and the poly(A) sequence of any depicted in SEQ ID NOs: 1384-1391 and FIG. 19. In some embodiments, the DNA construct comprises in 5′ to 3′ order: (i) a 5′ ligation sequence of SEQ ID NO: 1351, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) a poly(A) sequence of 165 adenine nucleotides, and (vi) a 3′ ligation sequence of SEQ ID NO: 1352.

In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) any 3′ UTR sequence provided herein, (vii) a poly(A) sequence of 165 adenine nucleotides, (viii) a 3′ ligation sequence of SEQ ID NO: 1352, and (ix) a 3′ ribozyme of SEQ ID NO:1350.

In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) mtRNR1-AES 3′ UTR of SEQ ID NO: 1354, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) mtRNR1-LSP1 3′ UTR of SEQ ID NO: 1355, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) AES-mtRNR1 3′ UTR of SEQ ID NO: 1356, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct includes in 5′ to 3′ order: (i) a 5′ ribozyme of SEQ ID NO:1349, (ii) a 5′ ligation sequence of SEQ ID NO: 1351, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) AES-hBg 3′ UTR of SEQ ID NO: 1357, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) FCGRT-hBg 3′ UTR of SEQ ID NO: 1358, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) 2hBG 3′ UTR of SEQ ID NO: 1359, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) HBA1 3′ UTR of SEQ ID NO: 1360, (vii) a poly(A) sequence of 165 adenine nucleotides, (viii) a 3′ ligation sequence of SEQ ID NO: 1352, and (ix) a 3′ ribozyme of SEQ ID NO:1350.

In other embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO: 1348 (a coxsackievirus B3 IRES), (iv) a polynucleotide sequence of interest, (v) a WPRE sequence of SEQ ID NO: 1353, (vi) a poly(A) sequence of 165 adenine nucleotides, (vii) a 3′ ligation sequence of SEQ ID NO: 1352, and (viii) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO: 1348 (a coxsackievirus B3 IRES), (iv) a polynucleotide sequence of interest, (v) a WPRE sequence of SEQ ID NO: 1353, (vi) any 3′ UTR sequence provided herein, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO: 1348 (a coxsackievirus B3 IRES), (iv) a polynucleotide sequence of interest, (v) a WPRE sequence of SEQ ID NO: 1353, (vi) mtRNR1-AES 3′ UTR of SEQ ID NO: 1354, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO: 1348 (a coxsackievirus B3 IRES), (iv) a polynucleotide sequence of interest, (v) a WPRE sequence of SEQ ID NO: 1353, (vi) mtRNR1-LSP1 3′ UTR of SEQ ID NO: 1355, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO: 1348 (a coxsackievirus B3 IRES), (iv) a polynucleotide sequence of interest, (v) a WPRE sequence of SEQ ID NO: 1353, (vi) AES-mtRNR1 3′ UTR of SEQ ID NO: 1356, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO: 1348 (a coxsackievirus B3 IRES), (iv) a polynucleotide sequence of interest, (v) a WPRE sequence of SEQ ID NO: 1353, (vi) AES-hBg 3′ UTR of SEQ ID NO: 1357, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO: 1348 (a coxsackievirus B3 IRES), (iv) a polynucleotide sequence of interest, (v) a WPRE sequence of SEQ ID NO: 1353, (vi) FCGRT-hBg 3′ UTR of SEQ ID NO: 1358, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO: 1348 (a coxsackievirus B3 IRES), (iv) a polynucleotide sequence of interest, (v) a WPRE sequence of SEQ ID NO: 1353, (vi) 2hBG 3′ UTR of SEQ ID NO: 1359, (vii) a poly(A) sequence of 165 adenine nucleotides, (viii) a 3′ ligation sequence of SEQ ID NO: 1352, and (ix) a 3′ ribozyme of SEQ ID NO:1350. In some embodiments, the DNA construct comprises in 5′ to 3′ order: (i) a 5′ ribozyme of SEQ ID NO:1349, (ii) a 5′ ligation sequence of SEQ ID NO: 1351, (iii) an IRES sequence of SEQ ID NO: 1348 (a coxsackievirus B3 IRES), (iv) a polynucleotide sequence of interest, (v) a WPRE sequence of SEQ ID NO: 1353, (vi) HBA1 3′ UTR of SEQ ID NO: 1360, (vii) a poly(A) sequence of 165 adenine nucleotides, (viii) a 3′ ligation sequence of SEQ ID NO: 1352, and (ix) a 3′ ribozyme of SEQ ID NO:1350.

In some embodiments, a linear RNA polynucleotide molecule (such as a linear icRNA) is produced by in vitro transcription of the DNA construct to generate a linearized ribozyme-RNA construct, and then self-cleavage of the ribozyme containing RNA construct by way of the ribozymes. In some embodiments, a linear icRNA as well as a corresponding circular icRNA is produced in a cell expressing the DNA construct (including vector described below). Standard methods for introducing the DNA construct into a cell can be used.

In some embodiments, a linear icRNA is produced by from in vitro transcription of the DNA construct (or vector described below) to produce a linear ribozyme containing RNA polynucleotide (also referred to as a ribozyme-RNA polynucleotide or construct), and then the linear ribozyme containing RNA polynucleotide self-cleaves at the ribozyme sequences to produce a linear icRNA. The linear icRNA can then be administered to a subject, and after introduction of the linear icRNA into the cell, the linear icRNA can ligate and circularize in the presences of RNA ligase to form circular icRNA. As such, the cell can contain a linear icRNA as well as the corresponding circular icRNA. Methods for administering the DNA construct are described below.

In some embodiments, a linear icRNA is produced by administering the DNA construct (or vector described below) into a subject, such that a cell in the subject produces a linear ribozyme containing RNA polynucleotide by transcription, and then the linear ribozyme containing RNA polynucleotide self-cleaves at the ribozyme sequences to produce a linear icRNA. The linear icRNA in the cell can ligate and circularize in the presences of RNA ligase to form circular icRNA. As such, the cell can contain a linear icRNA as well as the corresponding circular icRNA. Methods for administering the DNA construct are described below.

Provided herein are linear RNA constructs comprising a ribozyme. In some embodiments, the linear RNA construct comprises a ribozyme at the 5′ end and a ribozyme at the 3′ end. Such linear RNA constructs can be referred to as linear or linearized ribozyme-RNA constructs. A linearized ribozyme-RNA construct can undergo self-cleavage to form a linear icRNA.

A linearized ribozyme-RNA construct can be produced from in vitro transcription of a linear or circular DNA construct described herein. In some embodiments, a linear linearized ribozyme-RNA construct is chemically or synthetically synthesized by standard method known in the art.

In some embodiments, when a linear or circular DNA construct described herein is introduced to a cell, the linearized ribozyme-RNA construct is produced by the cell. Furthermore, linear and circular icRNAs can be formed by the cell.

In some embodiments, the linearized ribozyme-RNA construct comprises in 5′ to 3′ order: a ribozyme (e.g., 5′ ribozyme), a ligation sequence (e.g., 5′ ligation sequence), an IRES sequence, a polynucleotide sequence of interest, a WPRE sequence, a poly(A) sequence, a ligation sequence (e.g., 3′ ligation sequence), and a ribozyme (e.g., 3′ ribozyme). In some embodiments, the DNA construct includes in 5′ to 3′ order: a ribozyme (e.g., 5′ ribozyme), a ligation sequence (e.g., 5′ ligation sequence), an IRES sequence, a polynucleotide sequence of interest, a WPRE sequence, a 3′ UTR sequence, a poly(A) sequence, a ligation sequence (e.g., 3′ ligation sequence), and a ribozyme (e.g., 3′ ribozyme).

In some embodiments, a ribozyme of the linearized ribozyme-RNA construct are selected from the group consisting a twister ribozyme, twister sister (TS) ribozyme, a hammerhead ribozyme, a hairpin ribozyme, a hepatitis delta virus (HDV) ribozyme, a Varkud satellite (VS) ribozyme, a glucosamine-6-phosphate (GimS) ribozyme, a pistol ribozyme, and a hatchet ribozyme. In some embodiments, the 5′ ribozyme and the 3′ ribozyme are the same type of ribozymes. In other embodiments, the 5′ ribozyme and the 3′ ribozyme are different types of ribozymes. In some embodiments, the 5′ ribozyme or the 3′ ribozyme is a P1 twister ribozyme, an equivalent, and variant thereof. In some embodiments, the 5′ ribozyme or the 3′ ribozyme is a P3 twister ribozyme, an equivalent, and variant thereof. Both of the 5′ and 3′ ribozymes can be P1 twister ribozymes. In some cases, both of the 5′ and 3′ ribozymes are P1 twister ribozymes. In some instances, the 5′ ribozyme sequence comprises the nucleic acid of SEQ ID NO: 1349, wherein the T nucleotides are U nucleotides in the RNA construct. The 5′ ribozyme sequence can comprise the sequence of SEQ ID NO: 1412. In some instances, the 3′ ribozyme sequence comprises the nucleic acid of SEQ ID NO: 1350 wherein the T nucleotides are U nucleotides in the RNA construct. The 3′ ribozyme sequence can comprise the sequence of SEQ ID NO: 1414. In some instances, the 5′ ligation sequence comprises the nucleic acid of SEQ ID NO: 1351 wherein the T nucleotides are U nucleotides in the RNA construct. In some instances, the 3′ ligation sequence comprises the nucleic acid of SEQ ID NO: 1352 wherein the T nucleotides are U nucleotides in the RNA construct.

In some embodiments, the IRES of the linearized ribozyme-RNA construct is any selected from the group consisting of a cricket paralysis virus IRES, a Homo sapiens IGF2 IRES, a hepatovirus A IRES, a hepatitis C virus H77 isolate IRES, a Homo sapiens FGF1 IRES, a bovine viral diarrhea virus 1 IRES, a human rhinovirus A89 IRES, a pan paniscus LIMA1, a human adenovirus 2 IRES, a Montana myotis leukoencephalitis virus IRES, a Homo sapiens RANBP3 IRES, a pestivirus giraffe 1 IRES, a Homo sapiens TGIF1 IRES, a human poliovirus 1 mahoney IRES, a foot-and-mouth disease virus type O IRES, an encephalomyocarditis virus 7A IRES, an encephalomyocarditis virus 6A IRES, an enterovirus 71 IRES, a coxsackievirus B3 IRES, and an IRES sequence provided in the sequence listing including in SEQ ID NOS: 1-1329, 1330-1348, and 1361-1391 and the figures such as FIGS. 17-19, wherein the T nucleotides are U nucleotides in the RNA construct. In some embodiments, the IRES sequence is a nucleic acid sequence having at least 90%, e.g., at least 90%, 91%, 92%, 93%, 94%, 95%, 96%, 97%, 98%, 99% or more, sequence identity to any one of SEQ ID NOS: 1-1329, 1330-1348, and 1361-1391 and any one shown in FIGS. 17-19, wherein the T nucleotides are U nucleotides in the RNA construct. In some embodiments, the WPRE sequence of the linearized ribozyme-RNA construct is the nucleic acid sequence of SEQ ID NO:1353, wherein the T nucleotides are U nucleotides in the RNA construct. In some embodiments, the WPRE sequence is a nucleic acid sequence having at least 90%, e.g., at least 90%, 91%, 92%, 93%, 94%, 95%, 96%, 97%, 98%, 99% or more sequence identity to of SEQ ID NO:1353, wherein the T nucleotides are U nucleotides in the RNA construct. In some embodiments, the 3′UTR sequence of the linearized ribozyme-RNA construct is any selected from the group consisting of an mtRNR1-AES 3′ UTR, an mtRNR1-LSP1 3′ UTR, an AES-mtRNR1 3′ UTR, an AES-hBg 3′ UTR, an FCGRT-hBg 3′ UTR, a 2hBg 3′ UTR, and a HBA1 3′ UTR, a 3′UTR sequence provided in the sequence listing including in SEQ ID NOS: 1354-1360 and 1384-1390 and the figures such as FIG. 19, wherein the T nucleotides are U nucleotides in the RNA construct. In some embodiments, the 3′UTR sequence is a nucleic acid sequence having at least 90%, e.g., at least 90%, 91%, 92%, 93%, 94%, 95%, 96%, 97%, 98%, 99% or more, sequence identity to any one of SEQ ID NOS: 1354-1360 and 1384-1390 and any one shown in FIG. 19. In some embodiments, the poly(A) sequence of the linearized ribozyme-RNA construct is a stretch or chain of about 5-500, 10-500, 20-500, 50-500, 70-500, 80-500, 90-500, 100-500, 110-500, 120-500, 130-500, 140-500, 150-500, 160-500, 165-500, 170-500, 180-500, 190-500, 200-500, 10-200, 20-200, 30-200, 40-200, 50-200, 60-200, 70-200, 80-200, 90-200, 100-200, 50, 120, 150, 165, 200, 500, or more adenine nucleotides.

In some embodiments, the linearized ribozyme-RNA construct comprises the IRES sequence, the WPRE sequence, and the poly(A) sequence of any depicted in SEQ ID NOs: 1381-1391 and FIG. 19, wherein the T nucleotides are U nucleotides in the RNA construct. In some embodiments, the linearized ribozyme-RNA construct comprises the IRES sequence, the WPRE sequence, a 3′ UTR sequence, and the poly(A) sequence of any depicted in SEQ ID NOs: 1384-1391 and FIG. 19, wherein the T nucleotides are U nucleotides in the RNA construct. In some embodiments, the linearized ribozyme-RNA construct comprises in 5′ to 3′ order: (i) a 5′ ligation sequence of SEQ ID NO: 1413, (ii) an IRES sequence of SEQ ID NO:1346 (encephalomycarditis virus 6A IRES), (iii) a polynucleotide sequence of interest, (iv) a WPRE sequence of SEQ ID NO: 1353, (v) a poly(A) sequence of 165 adenine nucleotides, and (vi) a 3′ ligation sequence of SEQ ID NO: 1415, wherein the T nucleotides are U nucleotides in the RNA construct.

In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) any 3′ UTR sequence provided herein, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct.

In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) mtRNR1-AES 3′ UTR of SEQ ID NO: 1354, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) mtRNR1-LSP1 3′ UTR of SEQ ID NO: 1355, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) AES-mtRNR1 3′ UTR of SEQ ID NO: 1356, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct includes in 5′ to 3′ order: (i) a 5′ ribozyme of SEQ ID NO:1349, (ii) a 5′ ligation sequence of SEQ ID NO: 1413, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) AES-hBg 3′ UTR of SEQ ID NO: 1357, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) FCGRT-hBg 3′ UTR of SEQ ID NO: 1358, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) 2hBG 3′ UTR of SEQ ID NO: 1359, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO:1346 (encephalomycarditis virus 6A IRES), (iv) a polynucleotide sequence of interest, (v) a WPRE sequence of SEQ ID NO: 1353, (vi) HBA1 3′ UTR of SEQ ID NO: 1360, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct.

In other embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO: 1348 (a coxsackievirus B3 IRES), (iv) a polynucleotide sequence of interest, (v) a WPRE sequence of SEQ ID NO: 1353, (vi) a poly(A) sequence of 165 adenine nucleotides, (vii) a 3′ ligation sequence of SEQ ID NO: 1415, and (viii) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO: 1348 (a coxsackievirus B3 IRES), (iv) a polynucleotide sequence of interest, (v) a WPRE sequence of SEQ ID NO: 1353, (vi) any 3′ UTR sequence provided herein, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO: 1348 (a coxsackievirus B3 IRES), (iv) a polynucleotide sequence of interest, (v) a WPRE sequence of SEQ ID NO: 1353, (vi) mtRNR1-AES 3′ UTR of SEQ ID NO: 1354, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO: 1348 (a coxsackievirus B3 IRES), (iv) a polynucleotide sequence of interest, (v) a WPRE sequence of SEQ ID NO: 1353, (vi) mtRNR1-LSP1 3′ UTR of SEQ ID NO: 1355, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO: 1348 (a coxsackievirus B3 IRES), (iv) a polynucleotide sequence of interest, (v) a WPRE sequence of SEQ ID NO: 1353, (vi) AES-mtRNR1 3′ UTR of SEQ ID NO: 1356, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO: 1348 (a coxsackievirus B3 IRES), (iv) a polynucleotide sequence of interest, (v) a WPRE sequence of SEQ ID NO: 1353, (vi) AES-hBg 3′ UTR of SEQ ID NO: 1357, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1413, (iii) an IRES sequence of SEQ ID NO: 1348 (a coxsackievirus B3 IRES), (iv) a polynucleotide sequence of interest, (v) a WPRE sequence of SEQ ID NO: 1353, (vi) FCGRT-hBg 3′ UTR of SEQ ID NO: 1358, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1415, (iii) an IRES sequence of SEQ ID NO: 1348 (a coxsackievirus B3 IRES), (iv) a polynucleotide sequence of interest, (v) a WPRE sequence of SEQ ID NO: 1353, (vi) 2hBG 3′ UTR of SEQ ID NO: 1359, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct. In some embodiments, the linearized ribozyme-RNA construct comprises in 5′ to 3′ order: (i) a 5′ ribozyme of SEQ ID NO:1349, (ii) a 5′ ligation sequence of SEQ ID NO: 1415, (iii) an IRES sequence of SEQ ID NO: 1348 (a coxsackievirus B3 IRES), (iv) a polynucleotide sequence of interest, (v) a WPRE sequence of SEQ ID NO: 1353, (vi) HBA1 3′ UTR of SEQ ID NO: 1360, (vii) a poly(A) sequence of 165 adenine nucleotides, (viii) a 3′ ligation sequence of SEQ ID NO: 1415, and (ix) a 3′ ribozyme of SEQ ID NO:1350, wherein the T nucleotides are U nucleotides in the RNA construct.

As described above, a linearized ribozyme-RNA construct that is a precursor form of a linearized ribozyme activated RNA construct includes two ligation sequences located at opposite ends of the linearized ribozyme-RNA construct (e.g., a 5′ ligation sequence and a 3′ ligation sequence), as well as two ribozyme sequences located at opposite ends of the linearized ribozyme-RNA construct (e.g., a 5′ ribozyme sequence and a 3′ ribozyme sequence). In some embodiments, the ligation sequence and ribozyme sequence are complementary. In some embodiments, the 5′ ligation sequence and 5′ ribozyme sequence are complementary. In some embodiments, the 3′ ligation sequence and 3′ ribozyme sequence are complementary. In some embodiments, the 5′ ligation sequence and 5′ ribozyme sequence are complementary, and the 3′ ligation sequence and 3′ ribozyme sequence are complementary. In some embodiments, the ligation sequence and ribozyme sequence are partially complementary. In some embodiments, the 5′ ligation sequence and 5′ ribozyme sequence are partially complementary. In some embodiments, the 3′ ligation sequence and 3′ ribozyme sequence are partially complementary. In some embodiments, the 5′ ligation sequence and 5′ ribozyme sequence are partially complementary, and the 3′ ligation sequence and 3′ ribozyme sequence are partially complementary. In some embodiments, the ligation sequence or a portion and the ribozyme sequence or a portion thereof hybridize together in a cell and/or in standard in vitro conditions. In some embodiments, the ligation sequence or a portion and the ribozyme sequence or a portion are at least 85%, e.g., at least 85%, 86%, 87%, 88%, 89%, 90%, 91%, 92%, 93%, 94%, 95%, 96%, 97%, 98%, 99% or more complementary over the length of one of the sequences. In some embodiments, the ligation sequence or a portion and the ribozyme sequence or a portion are 100% complementary over the length of at least one of the sequences. In some embodiments, the 5′ ligation sequence or a portion and the 5′ ribozyme sequence or a portion thereof hybridize together in a cell and/or in standard in vitro conditions. In some embodiments, the 5′ ligation sequence or a portion and the 5′ ribozyme sequence or a portion are at least 85%, e.g., at least 85%, 86%, 87%, 88%, 89%, 90%, 91%, 92%, 93%, 94%, 95%, 96%, 97%, 98%, 99% or more complementary over the length of one of the sequences. In some embodiments, the 5′ ligation sequence or a portion and the 5′ ribozyme sequence or a portion are 100% complementary over the length of at least one of the sequences. In some embodiments, the 3′ ligation sequence or a portion and the 3′ ribozyme sequence or a portion thereof hybridize together in a cell and/or in standard in vitro conditions. In some embodiments, the 3′ ligation sequence or a portion and the 3′ ribozyme sequence or a portion are at least 85%, e.g., at least 85%, 86%, 87%, 88%, 89%, 90%, 91%, 92%, 93%, 94%, 95%, 96%, 97%, 98%, 99% or more complementary over the length of one of the sequences. In some embodiments, the 3′ ligation sequence or a portion and the 3′ ribozyme sequence or a portion are 100% complementary over the length of at least one of the sequences. In some instances, the 5′ ligation sequence is at least 85%, e.g., at least 85%, 86%, 87%, 88%, 89%, 90%, 91%, 92%, 93%, 94%, 95%, 96%, 97%, 98%, 99% or more complementary to the 5′ ribozyme sequence. In some instances, the 3′ ligation sequence is at least 85%, e.g., at least 85%, 86%, 87%, 88%, 89%, 90%, 91%, 92%, 93%, 94%, 95%, 96%, 97%, 98%, 99% or more complementary to the 3′ ribozyme sequence. In some embodiments, the linearized ribozyme activated RNA construct is found in a structure where the 5′ ligation sequence and the 5′ ribozyme sequence hybridize (see, for example, the linearized ribozyme-RNA construct in FIGS. 1A and 7A). In some embodiments, the linearized ribozyme activated RNA construct is found in a structure where the 3′ ligation sequence and the 3′ ribozyme sequence hybridize (see, for example, the linearized ribozyme-RNA construct in FIGS. 1A and 7A). In some embodiments, the 5′ ribozyme sequence or the 3′ ribozyme sequence comprises the nucleic acid sequence of SEQ ID NO: 1349. In some embodiments, the 5′ ribozyme sequence or the 3′ ribozyme sequence comprises a nucleic acid sequence having at least 90%, e.g., at least 90%, 91%, 92%, 93%, 94%, 95%, 96%, 97%, 98%, 99% or more sequence identity to SEQ ID NO: 1349. In some cases, the 5′ and 3′ ribozyme sequences both comprise the nucleic acid sequence of SEQ ID NO: 1349. In several embodiments, the 5′ ribozyme sequence or the 3′ ribozyme sequence comprises the nucleic acid sequence of SEQ ID NO: 1350. In various embodiments, the 5′ ribozyme sequence or the 3′ ribozyme sequence comprises a nucleic acid sequence having at least 90%, e.g., at least 90%, 91%, 92%, 93%, 94%, 95%, 96%, 97%, 98%, 99% or more sequence identity to SEQ ID NO: 1350. In some instances, the 5′ and 3′ ribozyme sequences both comprise the nucleic acid sequence of SEQ ID NO: 1350. The 5′ and 3′ ribozyme sequences can comprise the same nucleic acid sequence. The 5′ and 3′ ribozyme sequences can comprise different nucleic acid sequences. In some embodiments, the 5′ ribozyme sequence is SEQ ID NO: 1349 and the 3′ ribozyme sequence is SEQ ID NO: 1350. In some instances, a portion of the 5′ ribozyme sequence is complementary to a portion the 5′ ligation sequence of a linearized ribozyme-RNA construct. In some instances, a portion of the 3′ ribozyme sequence is complementary to a portion the 3′ ligation sequence of a linearized ribozyme-RNA construct. In some embodiments, the 5′ ligation sequence or the 3′ ligation sequence includes the nucleic acid sequence of SEQ ID NO: 1413. In some embodiments, the 5′ ligation sequence or the 3′ ligation sequence includes a nucleic acid sequence having at least 90%, e.g., at least 90%, 91%, 92%, 93%, 94%, 95%, 96%, 97%, 98%, 99% or more sequence identity to SEQ ID NO: 1413. In some cases, the 5′ and 3′ ligation sequences both comprise the nucleic acid sequence of SEQ ID NO: 1413. In several embodiments, the 5′ ligation sequence or the 3′ ligation sequence includes the nucleic acid sequence of SEQ ID NO: 1415. In various embodiments, the 5′ ligation sequence or the 3′ ligation sequence comprises a nucleic acid sequence having at least 90%, e.g., at least 90%, 91%, 92%, 93%, 94%, 95%, 96%, 97%, 98%, 99% or more sequence identity to SEQ ID NO: 1415. In some instances, the 5′ and 3′ ligation sequences both comprise the nucleic acid sequence of SEQ ID NO: 1415. The 5′ and 3′ ligation sequences can comprise the same nucleic acid sequence. The 5′ and 3′ ligation sequences can have the same nucleic acid sequence. The 5′ and 3′ ligation sequences can comprise different nucleic acid sequences. The 5′ and 3′ ligation sequences can have different nucleic acid sequences. In some embodiments, the 5′ ligation sequence is SEQ ID NO: 1413 and the 3′ ligation sequence is SEQ ID NO: 1415. In some instances, a portion of the 5′ ligation sequence is complementary to a portion the 3′ ligation sequence of a linearized ribozyme-RNA construct.

Concurrently, to enable compatibility between persistence of expression and immunogenicity, the LORAX protein engineering platform was developed, the platform can be applied iteratively to tackle particularly challenging multiplexed protein engineering tasks by exploring huge swaths of combinatorial mutation space unapproachable using previous techniques. Furthermore, while this methodology is particularly suited to the unique challenges of protein de-immunization, it is also applicable to any potential protein engineering goal, so long as there exists an appropriate screening procedure to select for the desired protein functionality.

Provided herein are in situ circularized RNAs (icRNAs) including circular icRNAs, linear icRNAs comprising hybridized ligation sequences, and linear ribozyme-RNA constructs comprising ribozymes as well as DNA constructs encoding such icRNAs and ribozyme-RNA constructs that are useful for applications such as, but not limited to, those based on RNA replacement, RNA editing, RNA regulation, RNA vaccines, protein production, and protein replacement. In some embodiments, the icRNAs are used in RNA-based therapies for the treatment and prophylactic treatment of a disease or condition in a subject, e.g., a human subject. In some embodiments, the linear icRNAs are used in RNA-based therapies for the treatment and prophylactic treatment of a disease or condition in a subject, e.g., a human subject. In some embodiments, the circular icRNAs are used in RNA-based therapies for the treatment and prophylactic treatment of a disease or condition in a subject, e.g., a human subject.

In some embodiments, linear icRNAs can be introduced or delivered to in vitro cells such as in vitro human cells. In certain embodiments, linear icRNAs can be introduced or delivered to ex vivo cells such as ex vivo human cells. In certain embodiments, linear icRNAs can be introduced or delivered to cells in vivo. In several embodiments, linear icRNAs can be introduced, delivered, or administered to a subject such as a human subject.

In some embodiments, linear ribozyme-RNA constructs can be introduced or delivered to in vitro cells such as in vitro human cells. In many embodiments, linear ribozyme-RNA constructs can be introduced or delivered to ex vivo cells such as ex vivo human cells. In certain embodiments, linear ribozyme-RNA constructs can be introduced or delivered to cells in vivo. In several embodiments, linear ribozyme-RNA constructs can be introduced, delivered, or administered to a subject such as a human subject.

In some embodiments, DNA constructs encoding linear icRNAs can be introduced or delivered to in vitro cells such as in vitro human cells. In many embodiments, DNA constructs encoding linear icRNAs can be introduced or delivered to ex vivo cells such as ex vivo human cells. In certain embodiments, DNA constructs encoding linear icRNAs can be introduced or delivered to cells in vivo. In several embodiments, DNA constructs encoding linear icRNAs can be introduced, delivered, or administered to a subject such as a human subject.

Looking ahead, in addition to its core utility in applications entailing transgene delivery, it is anticipated that icRNAs will be particularly useful in scenarios where a longer duration pulse of protein production is required. These applications include, but are not limited to, for instance, (1) epigenome engineering, (2) cellular reprogramming, (3) transient healing, and (4) rejuvenation applications.

Additionally, icRNA activity and versatility could be further bolstered by: inserting into a construct described herein a self-amplifying RNA payloads and/or inserting a Gag RNA fragment such as SEQ ID NO: 1416, wherein T nucleotides are U nucleotides to enable the packaging of icRNAs into viral like particles via co-expression of Gag protein. Taken together, it is expected that the simple and scalable icRNA methodology could have broad utility in basic science and therapeutic applications.

The RNA constructs of the disclosure have great utility in gene therapy space to treat widespread diseases. In both type 1 and type 2 diabetes, insulin production is limited and therefore patients commonly must exogenously administer insulin when their blood glucose levels rise. The inducible ribozyme-mediated RNA-construct (icRNA) system described herein can be adapted to contain two halves of the insulin gene fused to intronic sequences. The two constructs are constitutively present in muscular tissue, but one half would only be transcribed upon additional of an aptamer-binding ligand such as a synthetic sugar. This would lead to the rapid upregulation of ribozyme-mediated hybridization and splicing to generate the full length, functional insulin protein. Upon degradation of the inducer, the one fusion fragment would become repressed and no more insulin would be produced until more of the ligand is administered, thus replacing the need for painful and burdensome exogenous administration of insulin with an endogenous system with precise temporal control.

The inducible ribozyme-mediated RNA-construct system described herein can be applied to generate an inducible gene expression system for the clotting factor IX for patients with hemophilia, the cystic fibrosis transmembrane conductance regulator protein for patients with cystic fibrosis, and the dystrophin protein for patients with Duchenne's muscular dystrophy. Broadly, any disease that results from a poorly expressed or mutated protein could benefit from the inducible ribozyme-mediated RNA-construct system disclosed herein. This includes, but is not limited to, disease such as β-thalassemia, severe combined immunodeficiency, spinal muscle atrophy, and age-related macular degeneration.

The inducible ribozyme-mediated RNA-construct system described herein can be broadly applied to gene therapies using the CRISPR/Cas toolset. CRISPR/Cas genome editing is highly adaptable and has been engineered to investigate and treat genetic diseases, cancers, immunological diseases, and infectious diseases. A major limitation in the translation of these therapies is the inability to control the expression of the Cas protein in vivo. The inducible ribozyme-mediated RNA-construct system described herein can overcome this limitation by fusing two portions of the Cas protein to intronic sequences in separate RNA constructs. One of these would be under the control of an inducer, making the expression of the Cas protein and its subsequent function completely inducible. This would enable precise control over the genome editing that is mediated by the CRISPR/Cas system. It is not limited to gene knockouts and could be broadly adapted to aid in controlled and inducible non-homologous end joining, homology directed repair, single-base exchanges, transcriptional regulation, base editors, PRIME editors, and RNA editing.

This system is further tunable as the AAV serotype used can be altered without having to alter the expression plasmid. Various serotypes can be used which specifically target tissues such as AAV8 for the liver, AAV9 for skeletal muscle, or AAV-PHP.B for the central nervous system. Furthermore, engineered recombinant AAVs which specifically target distinct cell types can also be utilized in addition to the broad range of serotypes already available to further enhance the specificity of the partial reprogramming system.

Utilizing the system described herein for the in vivo control of reprogramming factors, the system can be harnessed for a broad range of applications.

Generally, transient expression of the Yamanaka factors in vivo has been demonstrated to ameliorate aging hallmarks. The system of the disclosure with OSKM and the 3′-UTR aptazyme could be packaged into an AAV, designed to either have broad tropism across the body, or targeted to a specific organ via an engineered AAV. This could then be administered to the subject and allowed to transduce its target organs for a short period of time. Subsequently, the ligand that is specific for the aptamer sequence could be administered at the desired dose and treatment regimen in order to achieve cyclic expression of OSKM. The physiological alterations induced by this approach could include a reduction in the DNA damage response associated with aging, downregulation of senescence and stress-related genes, and alterations to the epigenetic modifications that occur with aging. These molecular alterations at the cellular level have important implications for reducing the systematic aging issues. Furthermore, in the context of specific diseases related to aging, such as Hutchinson-Gilford Progeria syndrome, this strategy can be an important therapeutic option to systematically reduce physiological hallmarks of aging while also prolonging the lifespan of those affected.

On the tissue-specific level, the system of the disclosure can demonstrate an important therapeutic benefit as engineering of the AAV capsid can be utilized for cell-specific targeting of the inducible-reprogramming strategy. In the central nervous system, transient expression of OSK could be utilized to restore youthful DNA methylation patterns and transcriptomes in the retinal ganglion cells in order to promote axonal regeneration after injury and promote vision restoration for the aging population or those afflicted with visual impairments such as glaucoma. Similarly, targeting the system of the disclosure to specific brain regions (e.g., hippocampus) can be an important tool for improving memory through specific targeting of dentate gyrus cells. In the cardiovascular system, targeting the system of the disclosure to cardiomyocytes can lead to dedifferentiation of these post-mitotic cells. This enabled regenerative capacity has the potential to broadly improve cardiac function with the potential to greatly improve cardiomyocyte recovery following traumatic events such as myocardial infarction. Administration of the inducible OSKM construct as described herein and then treating the afflicted individual with the inducing ligand could drastically improve recovery from cardiovascular events. Furthermore, myofiber- and liver-specific transient expression mediated by the system of the disclosure has the ability to promote muscle regeneration in vivo, which has broad implications in both the aging and diseased setting.

Aside from induced AAV-aptazyme mediated expression of OSKM, the methods and compositions of the disclosure can be applied to other reprogramming transcription factors (TFs) as well. Depending on the outcome desired, TFs could be delivered either individually or in combination with either matching aptazyme sequences or separate aptazymes to enable temporal control of gene expression. These engineered TFs can be applied to the healthy and diseased settings with even broader implications for the whole field of regenerative medicine. The iAAV-partial reprogramming approach of the disclosure has broad applications across a diverse array of organ systems and disease settings.

RNA is inherently transient and this transience impacts their activity both as an interacting moiety as well as a template. Circularization of RNA polynucleotides improves their persistence, however simple and scalable approaches to achieve the same are lacking. Utilizing autocatalytic RNA circularization as described herein, the disclosure provides compositions and methods of in situ circularized RNAs (icRNAs) for durable protein translation. Specifically, an in vitro transcribed linear RNA that bears an internal ribosome entry site coupled to a messenger RNA of interest that is in turn flanked by ribozymes is provided. Once transcribed, the flanking twister ribozymes rapidly self-cleave, enabling hybridization of the complementary ligation stems to one another to generate circular RNAs. For example, delivery of linear RNAs into cells yields in situ circularized molecules upon autocatalytic cleavage of the ribozymes that leave termini which are ligated by endogenous RNA ligases. This scalable icRNA system has broad utility in basic science and therapeutic applications.

Compositions herein can be used to treat a disease or condition in a subject. For example, a ribozyme-activated RNA construct (e.g., a linear icRNA or a circular icRNA) of the disclosure can be administered to treat a disease described herein.

A pharmaceutical composition can comprise a first active ingredient. The first active ingredient can comprise a ribozyme-activated RNA construct of the disclosure. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient.

A composition described herein can compromise an excipient. In some cases, an excipient can comprise a pharmaceutically acceptable excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some cases, a carrier or a diluent can comprise an excipient. In some cases, a carrier or diluent can comprise a water, a salt solution (e.g., a saline), an alcohol or any combination thereof.

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent or any combination thereof.

In some cases, an excipient can be a buffering agent. Non-limiting examples of suitable buffering agents can include sodium citrate, magnesium carbonate, magnesium bicarbonate, calcium carbonate, and calcium bicarbonate. Other buffering agent include, but are not limited to, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some cases, an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include, but not limited to, EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some cases, a pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The binders that can be used in a pharmaceutical formulation can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some cases, a pharmaceutical formulation can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some cases, a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isomorphous silicate, and microcrystalline cellulose as high HLB emulsifier surfactants.

In some cases, a pharmaceutical formulation can comprise a disintegrant as an excipient. In some cases, a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some cases, a disintegrant can be an effervescent disintegrant. Non-limiting examples of suitable effervescent disintegrants can include sodium bicarbonate in combination with citric acid, and sodium bicarbonate in combination with tartaric acid.

In some cases, an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some cases, a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some cases, an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

A composition may comprise a combination of the active agent, e.g., a ribozyme-activated RNA construct of the disclosure, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

In the instance where the ribozyme-activated RNA construct, linear icRNA or circular icRNA are present in an RNA form (vs. a DNA encoding such construct), the preparation may include suitable RNAse inhibitors. Such RNAse inhibitors can prevent degradation of the constructs prior to use.

In some embodiments, a pharmaceutical composition can be formulated in milligrams (mg), milligram per kilogram (mg/kg), copy number, or number of molecules. In some cases, a composition can comprise about 0.01 mg to about 2000 mg of the active agent. In some cases, a composition can comprise about: 0.01 mg, 0.1 mg, 1 mg, 10 mg, 100 mg, 500 mg, 1000 mg, 1500 mg, or about 2000 mg of the active agent.

The terms “subject,” “host,” “individual,” and “patient” may be used interchangeably herein to refer to any organism eukaryotic or prokaryotic. In some cases, “subject” may refer to an animal, such as a mammal. A mammal can be administered a ribozyme-activated RNA construct of the disclosure or composition as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder. In other embodiments, a subject has or is suspected of having a disease or disorder associated with aberrant protein expression or protein activity. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

In some embodiments, a method of treating a human in need thereof can comprise administering to the human a ribozyme-activated RNA construct of the disclosure. In some embodiments, compositions herein can be used to treat disease and conditions. A disease or condition can comprise a neurodegenerative disease, a muscular disorder, a metabolic disorder, an ocular disorder, or any combination thereof. The disease or condition can comprise cystic fibrosis, albinism, alpha-1-antitrypsin deficiency, Alzheimer disease, Amyotrophic lateral sclerosis (ALS), Asthma, β-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Parkinson's disease, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, various forms of cancer (e.g. BRCA1 and 2 linked breast cancer and ovarian cancer). In some cases, a disease or condition can comprise Mucopoysaccharidosis type I (MPSI). In some cases, the MPSI can comprise Hurler syndrome, Hurler-Scheie syndrome, Scheie syndrome, or any combination thereof. The disease or condition can comprise a muscular dystrophy, an omithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, or any combination thereof.

In some embodiments, naked RNA constructs can be delivered to cells or subjects. In other embodiments, a vector can be employed to deliver a ribozyme-activated RNA construct of the disclosure. A vector can comprise DNA, such as double stranded DNA or single stranded DNA. A vector can comprise RNA. In some cases, the RNA can comprise one or more base modifications. The vector can comprise a recombinant vector. In some cases, the vector can be a vector that is modified from a naturally occurring vector. The vector can comprise at least a portion of a non-naturally occurring vector. Any vector can be utilized. In some cases, the vector can comprise a viral vector, a liposome, a nanoparticle, an exosome, an extracellular vesicle, or any combination thereof. In some embodiments, plasmid vectors can be prepared from commercially available vectors. In other embodiments, viral vectors can be produced from baculoviruses, retroviruses, adenoviruses, AAVs, or a combination thereof. Examples of viral vectors include retroviral vectors, adenovirus vectors, adeno-associated virus vectors, alphavirus vectors and the like. In one embodiment, the viral vector is a lentiviral vector. Infectious tobacco mosaic virus (TMV)-based vectors can be used to manufacturer constructs (e.g., icRNAs) in tobacco leaves. Alphavirus vectors, such as Semliki Forest virus-based vectors and Sindbis virus-based vectors, have also been developed for use in gene therapy and immunotherapy. Such vectors can remain episomally. In aspects where gene transfer is mediated by a retroviral vector, a vector construct can refer to the polynucleotide comprising the retroviral genome or part thereof, and a gene of interest. In some cases, a vector can contain both a promoter and a cloning site into which a polynucleotide (e.g., a ribozyme-activated RNA construct) can be operatively linked. Such vectors are capable of transcribing RNA in vitro or in vivo and are commercially available. In some cases, a viral vector can comprise an adenoviral vector, an adeno-associated viral vector (AAV), a lentiviral vector, a retroviral vector, a portion of any of these, or any combination thereof. In some cases, a nanoparticle vector can comprise a polymeric-based nanoparticle, an aminolipid based nanoparticle, a metallic nanoparticle (such as gold-based nanoparticle), a portion of any of these, or any combination thereof. In some cases, a vector can comprise an AAV vector. A vector can be modified to include a modified VP1 protein (such as an AAV vector modified to include a VP1 protein). An AAV can comprise a serotype—such as an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, a derivative of any of these, or any combination thereof.

In some embodiments, a vector can comprise a nucleic acid that encodes a linear precursor of a ribozyme-activated RNA construct of the disclosure. In some embodiments, a nucleic acid can comprise a linear precursor of a ribozyme-activated RNA construct of the disclosure. In some cases, the nucleic acid can be double stranded. In some instances, the nucleic acid can be DNA or RNA. In some cases, a nucleic acid can comprise more than one copy of a ribozyme-activated RNA construct of the disclosure. For example, a nucleic acid can comprise 2, 3, 4, 5, or more copies of a ribozyme-activated RNA construct of the disclosure. In some instances, the nucleic acid can comprise a U6 promoter, a CMV promotor or any combination thereof.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and ‘Vector” can be used interchangeably. However, the disclosure is intended to include such other forms of expression vectors, such as viral vectors (e.g., replication defective retroviruses, adenoviruses and adeno-associated viruses), which serve equivalent functions. Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Typically, the vector or plasmid contains sequences directing transcription and translation of a gene fragment, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant coding regions in the desired host cell are numerous and familiar to those skilled in the art. Virtually any promoter capable of driving these genetic elements is suitable for use in the disclosure. For example, a pol III promoter, a U6 promoter, a CMV promoter, a T7 promoter, an H1 promoter, can be used to drive expression. Termination control regions may also be derived from various genes native to the preferred hosts.

Administration of a ribozyme-activated RNA construct of the disclosure can be effected in one dose, continuously or intermittently throughout the course of treatment. Methods of determining the most effective means and dosage of administration can vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can vary and depend on the disease or condition. Routes of administration can vary with the composition used for treatment, the purpose of the treatment, the health condition or disease stage of the subject being treated, and target cell or tissue. Non-limiting examples of routes of administration include oral administration, nasal administration, injection, and topical application.

Administration can refer to methods that can be used to enable delivery of compounds or compositions to the desired site of biological action (such as DNA constructs, viral vectors, or others). These methods can include topical administration (such as a lotion, a cream, an ointment) to an external surface of a surface, such as a skin. These methods can include parenteral administration (including intravenous, subcutaneous, intrathecal, intraperitoneal, intramuscular, intravascular or infusion), oral administration, inhalation administration, intraduodenal administration, and rectal administration. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition. In some cases, a cosmetic professional can administer the composition.

Administration or application of a composition disclosed herein can be performed for a treatment duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration or application of compositions disclosed herein can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a composition can be administered or applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

### Kits

Kits and articles of manufacture are also described herein that contain ribozyme-mediated RNA-constructs. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more RNA fusion constructs described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### EXAMPLES

### Example 1: Extensive In Vivo and In Vivo Protein Translation Via In Situ Circularized RNAs

Cell culture: HEK293T and HeLa cells were cultured in DMEM supplemented with 10% FBS and 1% Antibiotic-Antimycotic (Thermo Fisher). K562 cells were cultured in RPMI supplemented with 10% FBS and 1% Antibiotic-Antimycotic (Thermo Fisher). All cells were cultured in an incubator at 37° C. and 5% CO₂.

DNA transfections were performed by seeding HEK293T cells in 12 well plates at 25% confluency and adding 1 μg of each DNA construct and 4 μL of Lipofectamine 2000 (Thermo Fisher). RNA transfections were performed by adding 1 μg of each RNA construct and 3.5 μL of Lipofectamine MessengerMax (ThermoFisher). Electroporations were performed in K562 cells using the SF Cell Line 4D-Nucleofector X Kit S (Lonza) per manufacturer's protocol.

In vitro transcription: DNA templates for generating desired RNA products were created by PCR amplification from plasmids or gBlock gene fragments (IDT) and purified using a PCR purification kit (Qiagen). Plasmids were then generated with these templates containing a T7 promoter followed by 5′ ribozyme sequence, a 5′ ligation sequence, an IRES sequence linked to the product of interest, a 3′ ligation sequence, a 3′ ribozyme sequence, and lastly a poly-T tail to terminate transcription. Linear RNA products were then produced using the HiScribe T7 Quick High Yield RNA Synthesis Kit (NEB) per manufacturer's protocol.

Flow cytometry experiments: To assess persistence of RNA constructs in vitro, HEK293T cells were transfected with circular or mutated GFP RNA and GFP intensity, defined as the median intensity of the cell population, was quantified over the next three days using a BD LSRFortessa Cell Analyzer.

Lipid nanoparticle formulations: (6Z,9Z,28Z,31Z)-heptatriaconta-6,9,28,31-tetraen-19-yl-4-(dimethylamino) butanoate (DLin-MC3-DMA) was purchased from BioFine International Inc. 1,2-distearoyl-sn-glycero-3-phosphocholine (DSPC) and 1,2-dimyristoyl-rac-glycero-3-methoxypolyethylene glycol-2000 (DMG-PEG-2000) were purchased from Avanti Polar Lipids. Cholesterol was purchased from Sigma-Aldrich. mRNA LNPs were formulated with DLin-MC3-DMA:cholesterol:DSPC:DMG-PEG at a mole ratio of 50:38.5:10:1.5 and a N/P ratio of 5.4. To prepare LNPs, lipids in ethanol and mRNA in 25 mM acetate buffer, pH 4.0 were combined at a flow rate of 1:3 in a PDMS staggered herringbone mixer (PMID: 23344179, 22475086). The dimensions of the mixer channels were 200 by 100 um, with herringbone structures 30 um high and 50 um wide. Immediately after formulation, 3-fold volume of PBS was added and LNPs were purified in 100 kDa MWCO centrifugal filters by exchanging the volume 3 times. Final formulations were passed through a 0.2 um filter. LNPs were stored at 4° C. for up to 4 days before use. LNP hydrodynamic diameter and polydispersity index were measured by dynamic light scattering (Malvern NanoZS Zetasizer). The mRNA content and percent encapsulation were measured with a Quant-it RiboGreen RNA Assay (Invitrogen) with and without Triton X-100 according to the manufacturer's protocol.

Animal experiments: All animal procedures were performed in accordance with protocols approved by the Institutional Animal Care and Use Committee of the University of California, San Diego. All mice were acquired from Jackson Labs.

To assess persistence of RNA constructs in vivo, 10 μg of circular GFP or mutated GFP mRNA-LNPs were injected retro-orbitally into C57BL/6J mice. After 3 days and 7 days, livers were isolated and placed in RNAlater (Sigma-Aldrich). RNA was later isolated using QIAzol Lysis Reagent and purified using RNeasy Mini Kit (Qiagen) according to the manufacturer's protocol. mRNA expression of circular RNA and GFP was assessed by RT-qPCR.

To investigate the ability of circular and mutated COVID RNA to elicit an immune response, Balb/c mice were injected intramuscularly into the gastrocnemius muscle with PBS, 0.2 μg of mutated or circular mRNA-LNPs, or 2 μg of mutated or circular mRNA-LNPs. Blood draws were performed on days 0, 9, and 21, serum was separated using blood collection tubes (Sarstedt), and antibody production was then assessed by a sandwich enzyme-linked immunosorbent assay (ELISA). ELISA was performed using the ELISA Starter Accessory Kit (Bethyl, E101) per manufacturer's instructions. Briefly, 96-well MaxiSorp well plates were coated with recombinant SARS-COV-2 Spike protein 51, Omicron variant (GenScript Biotech) diluted in 1× coating buffer (Bethyl) to a concentration of 2 μg/mL overnight at 4 C. Plates were washed five times with 1× washing buffer (Bethyl), followed by the addition of 1× blocking buffer for 1 hour at RT. Samples were diluted 1:10 in sample/conjugate diluent (Bethyl) and added to the plate for 2 hours at RT. Sample/conjugate diluent was used as a blank. Plates were washed five times with 1× washing buffer and incubated in secondary antibody (horseradish peroxidase (HRP)-conjugated goat anti-mouse IgG antibody, Southern Biotech 1036-05, diluted 1:5000 in sample/conjugate diluent) for 1 hour at RT. After five washes, 50 μL/well TMB One Component HRP Microwell Substrate (Bethyl) was added and incubated for 15 min at RT in the dark. 50 μL/well of 0.2M H₂SO₄ was then added to terminate color development and absorbance was measured at 450 nm in a SpectraMax iD5 Multi-Mode Microplate Reader (Molecular Devices).

Identification of SpCas9 MHC binding epitopes: Two approaches were used to identify MHC binding epitopes. First, large amounts of available sequencing data were analyzed to identify low-frequency single nucleotide polymorphism, which represent mutational changes that are unlikely to induce non-functional variants. Secondly, potential mutations were screened in silico using the netMHC epitope prediction software. Using these strategies, 23 different mutations across 17 immunogenic epitopes were identified.

Identification of HPRT1 Guide: The lentiCRISPR-v2 plasmid (Addgene #52961) was first digested with Esp3I and a guide targeting the HPRT1 gene was cloned in via Gibson assembly. After lentivirus production, HeLa cells were seeded at 25% confluency in 96 well plates and transduced with virus (lentiCRISPR-v2 with or without HPRT1 guide) and 8 μg/mL polybrene (Millipore). Virus was removed the next day and 2.5 μg/mL puromycin was added to remove cells that did not receive virus two days later. After 2 days of puromycin selection, 0-14 μg/mL 6-TG was added. After 5 days, cells were stained with crystal violet, solubilized using 1% sodium dodecyl sulfate, and absorbance was measured at 595 nm on a plate reader. 6 μg/mL was chosen due to the lack of cells in the negative control.

Generation of variant Cas9 library: Cas9 variant sequences were generated by separating the full-length gene sequence into small sections, where each section contained wildtype or variant Cas9 sequences. gBlocks were PCR amplified and blocks annealed together, yielding a final library size of about 1.5 million elements. The lentiCRISPR-v2 plasmid containing the HPRT1 guide was digested with BamHI and XbaI and Gibson assembly was used to clone elements into the vector. The Gibson reactions were then transformed into electrocompetent cells and cultured at 37C overnight. Plasmid DNA was isolated using the Qiagen Plasmid Maxi Kit and library coverage was estimated by calculating the number of colonies found on LB-carbencillin plates. DNA was then used to create lentivirus containing the variant Cas9 library.

Cas9 Sreen: HeLa cells were seeded in 15 15-cm plates and transduced with virus containing the variant Cas9 library and 8 μg/mL polybrene. Media was changed the next day and 2.5 μg/mL puromycin was added to remove cells that did not receive virus two days later. 6 μg/mL 6-TG was added to media once cells reached 90% confluency. Media was changed every other day for ten days to allow for selection of cells containing functional Cas9 variants. After ten days, cells were lifted from the plates and DNA was isolated using the DNeasy Blood & Tissue Kit per manufacturer's protocol.

Nanopore Sequencing: Pre-screen analysis of the Cas9 variant library elements was performed by amplifying the sequence from the plasmid. 1 μg of the variant Cas sequences was used for library preparation using the Ligation Sequencing Kit (Oxford Nanopore Technologies, SQK-LSK109) per manufacturer's instructions. DNA was then loaded into a MinION flow cell (Oxford Nanopore Technologies, R9.4.1). Post-screen analysis of library elements was performed by amplifying the Cas9 sequences from 75 μg of genomic DNA. 1 μg of the variant Cas sequences was similarly prepared using the Ligation Sequencing Kit and sequenced on a MinION flow cell.

HDR validation: Lentivirus was produced from a plasmid containing a GFP sequence with a stop codon and 68 bp AAVS1 fragment and used to transduce HEK293T cells. After puromycin selection to create a stable line, cells were transduced with lentiCRISPR-v2 plasmids containing both variant Cas9 sequences and a guide targeting the AAVS locus and a GFP repair donor plasmid or separate plasmids containing Cas9 variant in a pZac 2.1 backbone, AAVS1 guide, and the GFP repair donor plasmid. After 3 days, FACS was performed and percent GFP positive was quantified.

Editing experiments: To validate variant Cas9 functional cutting, variant Cas9 and guides were transfected into HEK293T cells. After two days, genomic DNA was isolated. Genomic DNA was also isolated after two days from K562 cells after electroporation. To assess mutated and circular zinc finger and Cas activity, HEK293T cells were transfected with RNA and guide RNA, in the case of Cas, and genomic DNA was isolated after three days.

Epigenome experiments: dCas9 and CRISPRoff experiments were performed by transfecting HEK293T cells with DNA or RNA and isolating RNA three days later. Silencing or activation of genes was assessed by qPCR.

Quantification of editing using NGS: After extraction of genomic DNA, PCR was performed to amplify the target site. Amplicons were then indexed using the NEBNext Multiplex Oligos for Illumina kit (NEB). Amplicons were then pooled and sequenced using a Miseq Nano with paired end 150 bp reads. Editing efficiency was quantified using CRISPResso2.

Lentivirus production: HEK293FT cells were seeded in 1 15-cm plate and transfected with 36 μL Lipofectamine 2000, 3 μg pMD2.G (Addgene #12259), 12 μg pCMV delta R8.2 (Addgene #12263), and 9 μg of the lentiCRISPR-v2 plasmid. Supernatant containing viral particles was harvested after 48 and 72 hours, filtered with 0.45 μm Steriflip filters (Millipore), concentrated to a final volume of 1 mL using an Amicon Ultra-15 centrifugal filter unit with a 100,000 NMWL cutoff (Millipore), and frozen at −80 C.

RT-qPCR: cDNA was synthesized from RNA using the Protoscript II First Strand cDNA Synthesis Kit (NEB). qPCR was performed using a CFX Connect Real Time PCR Detection System (Bio-Rad). All samples were run in triplicates and results were normalized against GAPDH expression.

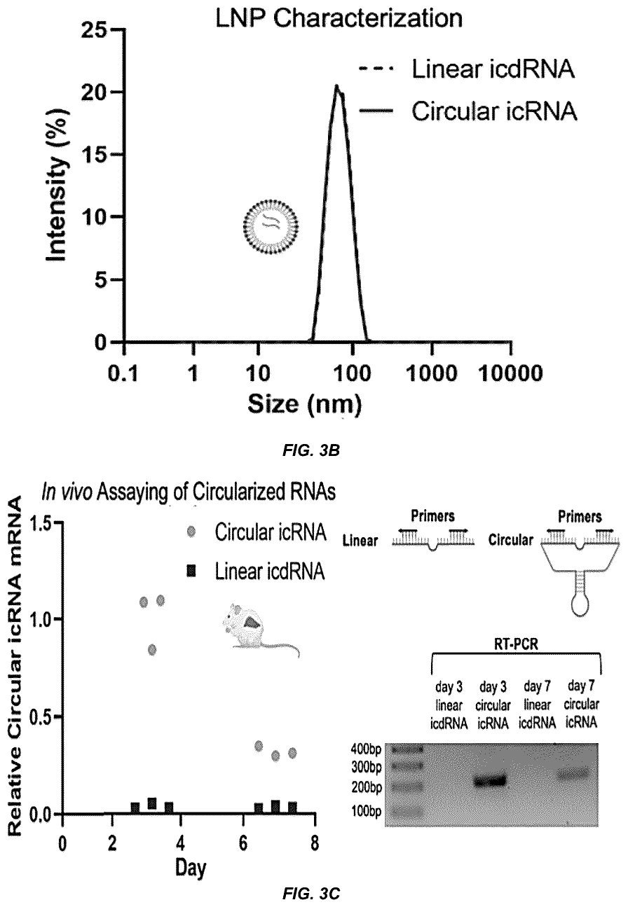

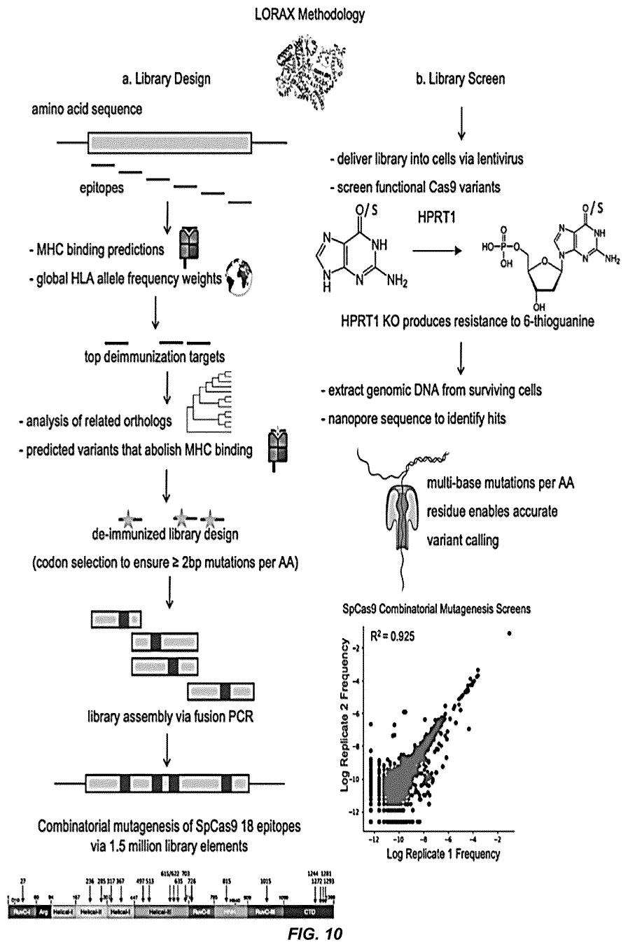

To engineer icRNAs, in vitro transcribed linear RNAs were generated that bear a twister ribozyme (also referred to as linearized ribozyme-RNAs) flanked internal ribosome entry site (IRES) coupled to a messenger RNA of interest (FIG. 1A). Once transcribed, the flanking twister ribozymes rapidly self-cleave, enabling hybridization of the complementary ligation stems to one another, and upon delivery into cells, these linear RNAs (also referred to herein as linear icRNAs) are then circularized in situ by the ubiquitous RNA ligase RtcB. To evaluate this approach, green fluorescent protein (GFP) translation was assayed via flow cytometry in the icRNA format in vitro in HEK293T cells. Towards this, a panel of IRES sequences, ligation stems, and 3′ untranslated regions (UTRs) (sequences shown in FIGS. 17A, 17B, and 17C) were screened to optimize protein translation (FIG. 3A). Next, studies were performed to investigate whether linear icRNAs were covalently circularized in cells upon delivery via RT-PCR by designing outward facing primers that selectively amplified only the circularized RNA molecules. As a negative control, linear in situ circularization defective RNAs (icdRNAs) were engineered by utilizing catalytically inactive mutants of the twister ribozymes. Indeed, only a PCR product for linear icRNAs was observed, confirming circularization to produce corresponding circular icRNAs (FIG. 1C). icRNA mRNA expression appeared to be quite consistent, reducing each day at roughly the rate of cell division (FIG. 3C). To investigate persistence of GFP expression over time, HEK293T cells were transfected with equal amounts of linear-GFP icRNA (linear GFP icRNA delivered into the cells and then circularized in the cell to produce circular GFP icRNAs) and linear-GFP icdRNA and performed FACS over three days. The former resulted in significantly robust and durable protein translation over time (FIG. 1B). Furthermore, persistence of circular-GFP icRNA (generated from the introduced linear GFP icRNAs) is significantly higher than commercial products (FIG. 3C). Ibis result was also validated via RT-PCR, as an increased persistence of GFP mRNA over time from cells transfected with linear icRNA compared to icdRNA was observed (FIG. 1B). To assess the durability of linear icRNA and corresponding circular icRNA in vivo, linear GFP icRNA and GFP icdRNA were encapsulated in lipid nanoparticles (LNPs) (FIG. 3A) and 10 μg were retro-orbitally injected into C57BL/6 mice. Persistence of circular GFP mRNA was observed in mice livers injected with linear icRNA encapsulated LNPs for at least seven days (FIG. 3A).

Studies were then performed to explore icRNA application in two distinct therapeutic transgene delivery contexts: one, to enable immunization via proteins delivered in the linear icRNA format, and two, to enable genome targeting via delivery of proteins. Towards the former, the production of IgG binding antibodies against SARS-CoV-2 Omicron variant spike protein was assessed in BALB/c mice via ELISA. Linear icRNAs and icdRNAs bearing the Omicron spike (K986P, V987P) protein were generated, encapsulated in LNPs, and delivered via a single intramuscular injection at doses of 0.2 μg linear icRNA or icdRNA/mouse and 2 μg icRNA or icdRNA/mouse. Robust induction of anti-spike IgG in the sera of animals receiving 2 μg linear icRNA at 3 weeks post injection compared to other groups (FIG. 1D) was confirmed. Towards the latter, linear zinc finger nuclease (ZFN) icRNAs and icdRNAs targeting the GFP and CCR5 genes were engineered, and again confirmed robust genome editing via icRNAs compared to icdRNAs via their delivery into HEK293T cells (FIG. 1E).

Spurred by these results studies were performed to explore if icRNAs could be used to deliver the CRISPR-Cas9 systems. It was conjectured that the prolonged expression via icRNAs could facilitate genome and especially epigenome targeting. However, this same feature of persistence could also aggravate immune responses in therapeutic settings as CRISPR systems are derived from prokaryotes. Thus, to enable compatibility between persistence of expression and immunogenicity, a methodology was developed to screen progressively deimmunized SpCas9 proteins by combinatorially mutating particularly immunogenic epitopes.

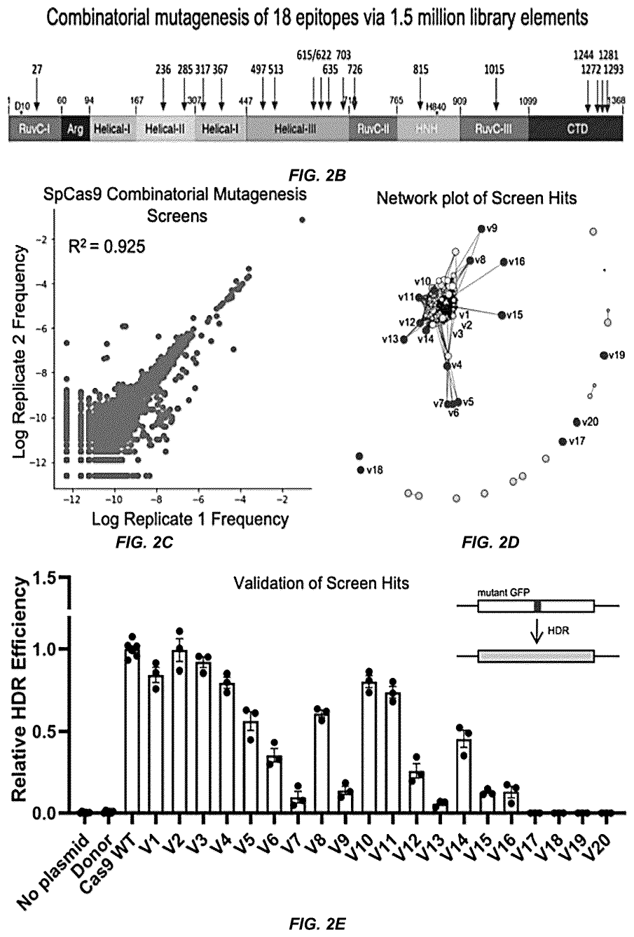

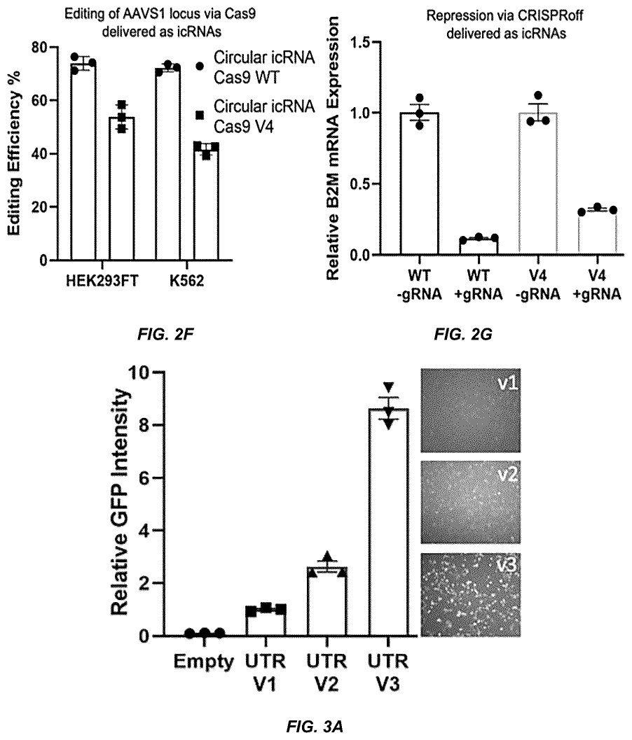

While variant library screening has proven to be an effective approach to protein engineering, applying it to deimmunization faces several technical challenges, namely: one, the need to mutate multiple sites simultaneously across the full length of the protein; two, reading out the associated combinatorial mutations scattered across large (>1 kb) regions of the protein via typical short read sequencing platforms; and three, engineering fully degenerate combinatorial libraries which can very quickly balloon to unmanageable numbers of variants. To overcome these challenges several methodological innovations were developed which, taken together, comprise a novel long range multiplexed (LORAX) protein engineering platform capable of screening millions of combinatorial variants simultaneously with mutations spread across the full length of arbitrarily large proteins (FIG. 2a).

Towards library design, in order to narrow down the vast mutational space associated with combinatorial libraries, an approach guided by evolution and natural variation was used. As de-immunizing protein engineering seeks to alter the amino acid sequence of a protein without disrupting functionality, it is extremely useful to narrow down mutations to those less likely to result in non-functional variants. To identify these mutants large amounts of sequencing data that was available was used to identify low-frequency SNPs that have been observed in natural environments. Such variants are likely to have limited effect on protein function, as highly deleterious alleles would likely be quickly selected out of natural populations and therefore not appear in sequencing data. Additionally, resulting potential mutations were evaluated for immunogenicity in silico using the netMHC epitope prediction software, in order to determine to what degree the mutations are likely to result in the de-immunization of that particular epitope. This is a useful step as some mutations may have little effect on overall immunogenicity. Screening thus filtered likely neutral amino acid substitutions, in combinatorial libraries should thus substantially increase the likelihood of functional hits with enough epitope variation to evade immune induction.

Next, to enable readout of screens, long-read nanopore sequencing was applied to measure the results of the screens of our combinatorial libraries. This circumvents the limit of short target regions and obviates the need for barcodes altogether by single-molecule sequencing of the entire target gene, enabling library design strategies which can explore any region of the protein in combination with any other region without any complicated cloning procedures required to facilitate barcoding. To date, the adoption of nanopore sequencing has been limited by its high error rate, around 95% accuracy per DNA base, as compared to established short read techniques which are multiple orders of magnitude more accurate. To address this challenge, libraries were designed such that each variant that was engineered would have multiple nucleotide changes for each single target amino acid change, effectively increasing the sensitivity of nanopore based readouts with increasing numbers of nucleotide changes per library member. The large majority of amino acid substitutions are amenable to a library design paradigm in which each substitution is encoded by two, rather than one, nucleotide changes, due to the degeneracy of the genetic code and the highly permissive third “wobble” position of codons.

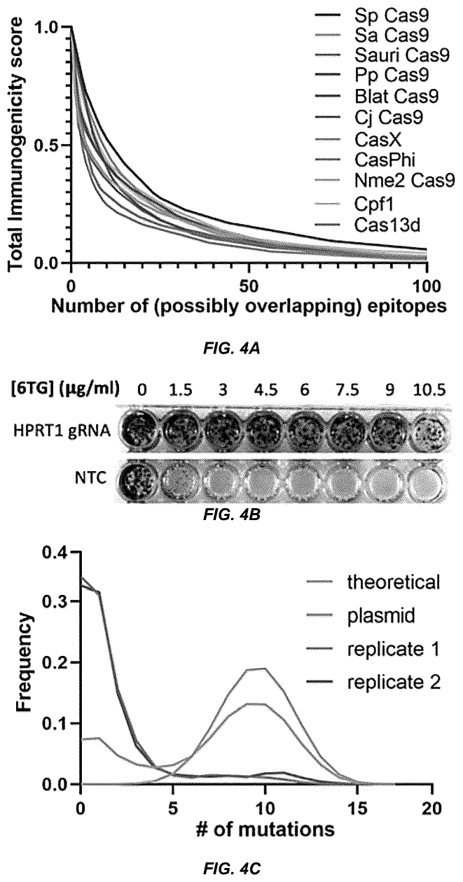

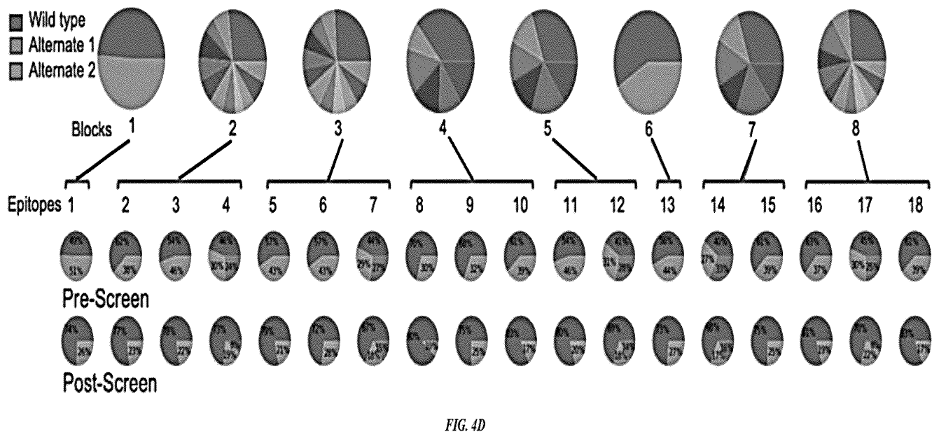

The scale of engineering which would be required to generate an effectively de-immunized Cas9 is not fully understood, as combinatorial de-immunization efforts at the scale of proteins thousands of amino acids long have not yet been possible. Therefore, to roughly estimate these parameters an immunogenicity scoring metric was developed, which takes into account all epitopes across a protein and the known diversity of MHC variants in a species weighted by population frequency to generate a single combined score representing the average immunogenicity of a full-length protein as a function of each of its immunogenic epitopes. Formally, this score is calculated as:

$${I}_{x} = \frac{{\sum }_{i}^{m} {\sum }_{j}^{n} {w}_{j} ( 1 - log ( {k}_{i j} * j ) )}{y}$$

where Iₓ=Immunogenicity score of protein x, i=epitopes, j=HLA alleles, j=allele specific standardization coefficient, wⱼ=HLA allele weights, kᵢⱼ=predicted binding affinity of epitope i to allele j, p=percentile rank of epitope i binding to allele j, and y=protein specific scaling factor. The overall effect of mutating the top epitopes in several Cas9 orthologs was then predicted (FIG. 4A). As might be expected, this analysis suggests that single-epitope strategies are woefully inadequate to de-immunize a whole protein, and also that there are diminishing returns as more and more epitopes are de-immunized. Our analysis suggests that it may require on the order of tens of de-immunized epitopes to make a significant impact on overall protein immunogenicity. The scale of engineering demanded by these immunological facts has previously been intractable, but by applying LORAX one could now make substantial steps, several mutations at a time, through the mutational landscape of the Cas9 protein.

Specifically, applying the procedure above, a library of Cas9 variants was designed based on the SpCas9 backbone containing 23 different mutations across 17 immunogenic epitopes (FIG. 2B). Combining these in all possible combinations yields a library of 1,492,992 unique elements. With this design, the library was constructed in a stepwise process. First, the full-length gene was broken up into short blocks of no more than 1000 bp, which overlap by 30 bp on each end. Each block is designed such that it contains no more than 3 or 4 target epitopes to mutagenize. With few epitopes per block and few variant mutations per epitope, it becomes feasible to chemically synthesize each combination of mutations for each block. Each of these combinations was then synthesized and mixed at equal ratios to make a degenerate block mix. This was repeated for each of the blocks necessary to complete the full-length protein sequence. Oxford Nanopore (ONT) MinION sequencing confirmed the majority of the pre-screened library consists of Cas9 sequences with significant numbers of mutations, with most falling into a broad peak between 6 and 14 mutations per sequence (FIG. 4C).

To identify functional variants still capable of editing DNA, a positive screen targeting the hypoxanthine phosphoribosyltransferase 1 (HPRT1) gene was designed and carried out. In the context of the screen, HPRT1 converts 6-thioguanine (6TG), an analogue of the DNA base guanine, into 6-thioguanine nucleotides that are cytotoxic to cells via incorporation into the DNA during S-phase. Thus, only cells containing functional Cas9 variants capable of disrupting the HPRT1 gene can survive in 6TG. To first identify the optimal 6TG concentration, HeLa cells were transduced with lentivirus particles containing wild-type Cas9 and either a HPRT1-targeting guide RNA (gRNA) or a non-targeting guide. After selection with puromycin, cells were treated with 6TG concentrations ranging from 0-14 μg/mL for one week. Cells were stained with crystal violet at the end of the experiment and imaged. 6 μg/mL was selected as all cells containing non-targeting guide had died while cells containing the HPRT1 guide remained viable (FIG. 4B).

To perform the screen, HeLa cells were transduced with lentiviral particles containing variant library along with the HPRT1-targeting gRNA at 0.3 MOI and at greater than 75-fold coverage of the library elements. Cells were selected using puromycin after two days and 6TG was added once cells reached 75% confluency. After two weeks, genomic DNA was extracted from remaining cells and full-length Cas9 sequences were PCR amplified. Nanopore-compatible sequencing libraries were prepared per manufacturer's instructions and sequenced on the MinION platform. This screening procedure was performed in two replicates.

Sequencing revealed that the library was significantly shifted in the mutation density distribution, suggesting that the majority of the library with large (>4) numbers of mutations resulted in non-functional proteins which were unable to survive the screen. Meanwhile, wild-type, single, and double mutants were generally enriched as these proteins proved more likely to retain functionality and pass through the screen (FIG. 4C). Additionally, the two independent replicates of the screen showed strong correlation (R²=0.925) providing further evidence of the robustness of the screen (FIG. 2C). In addition, the change in overall frequency of mutations in the pre- and post-screen libraries was analyzed to see if a pattern of mutation effects could be inferred. Although the wild-type allele was enriched at every site in the post-screen sequences, nearly every site retained a significant fraction of mutated alleles, suggesting that the mutations, at least individually, are fairly well-tolerated and do not disrupt Cas9 functionality (FIG. 4D).

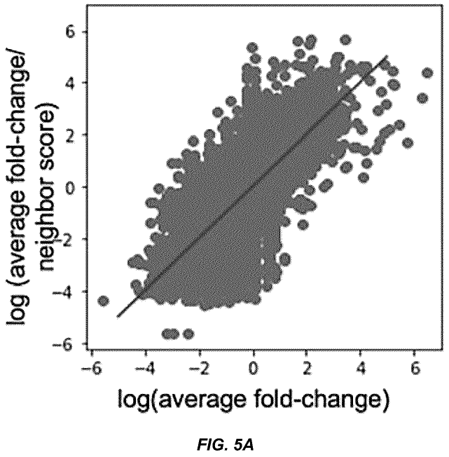

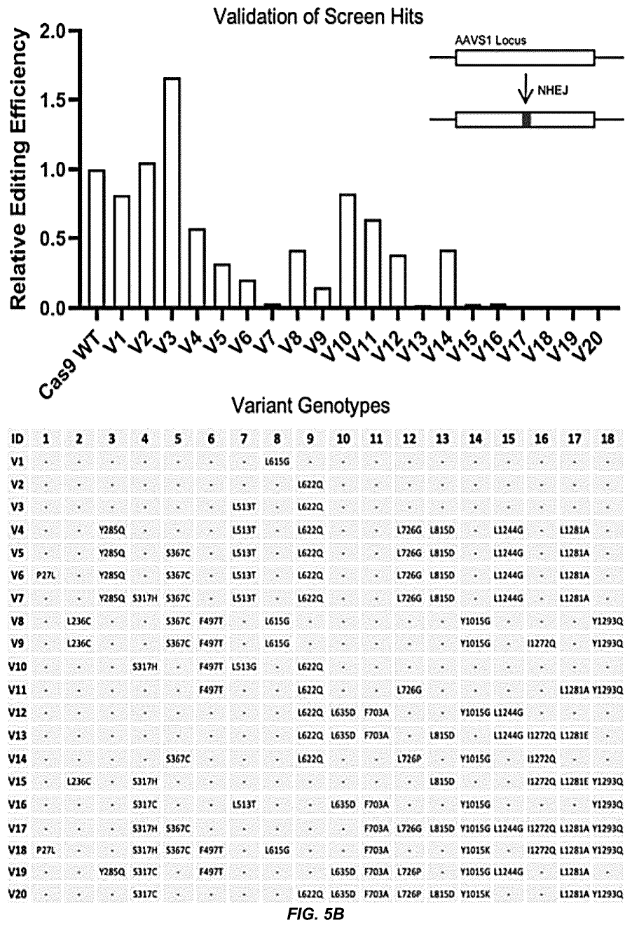

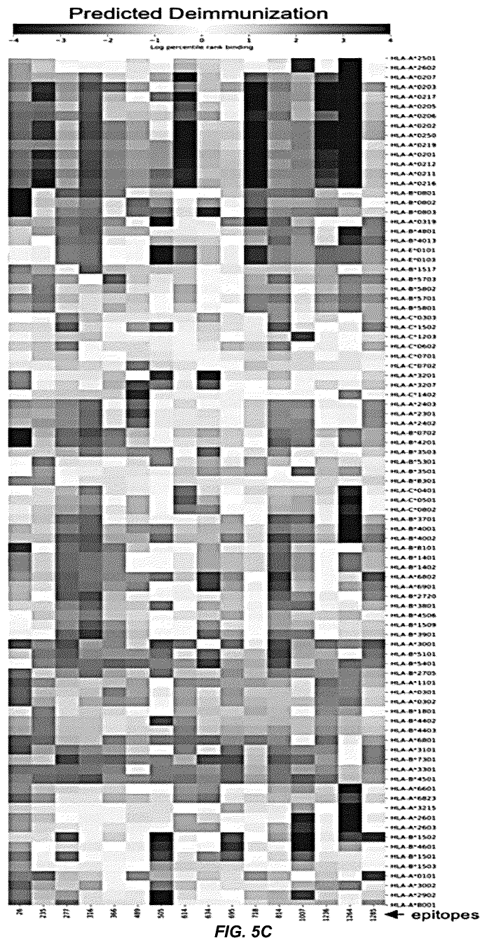

In order to select hits from the screen for downstream validation and analysis, a method for differentiating high-support hits likely to be real from noise-driven false positive hits was devised. To do this it was hypothesized that the fitness landscape of the screen mutants is likely to be smooth, i.e. variants that contain similar mutations are more likely to have similar fitnesses in terms of editing efficiency compared to randomly selected pairs. This was confirmed by computing a predicted screen score for each variant based on a weighted regression of its nearest neighbors in the screen. This metric correlates well with the actual screen scores and approaches the screen scores even more closely as read coverage increases. This provides good evidence that the fitness landscape is indeed somewhat smooth (FIG. 5A). Next, a network analysis was performed to try to differentiate noise-driven hits from bona fide hits by looking at the degree of connectivity with other hits. The rationale here is that because the fitness landscape is smooth, real hits should reside in broad fitness peaks including many neighbors that also show high screen scores, whereas hits that are less supported by near neighbors are more likely to be spurious as they represent non-smooth fitness peaks (FIG. 2D).

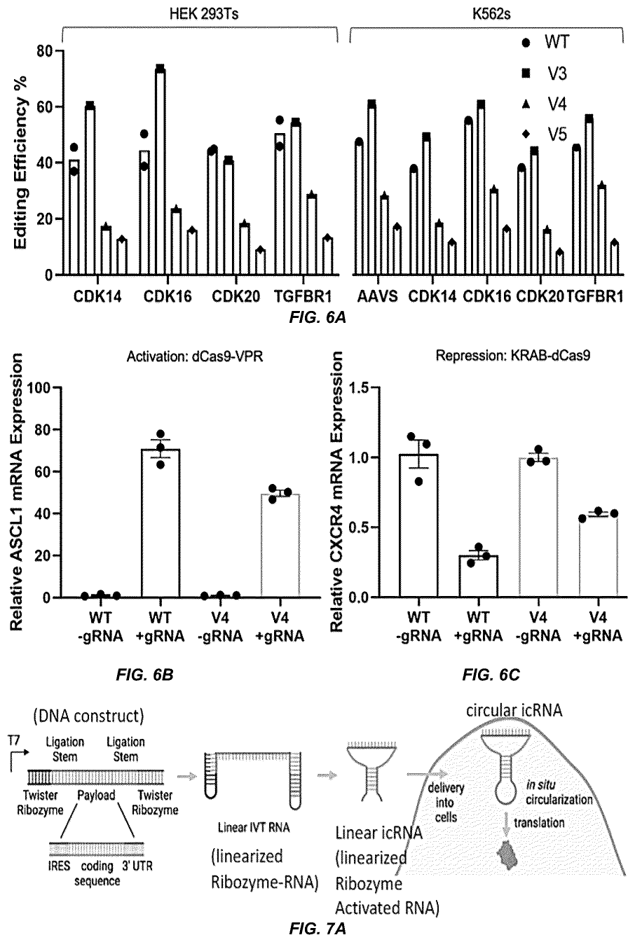

To validate and characterize hits from the screen, 20 variants (V1-20) were constructed (FIG. 5B) and applied to two independent methods to quantify editing of the de-immunized Cas9 variants. First, a gene-rescue experiment was performed using low frequency homology directed repair (HDR) to repair a genetically encoded broken green fluorescent protein (GFP) gene. (FIG. 2e); and second, editing by genomic DNA extraction and Illumina next generation sequencing (NGS) was quantified using the CRISPResso2 package (FIG. 5B). Variants connected to neighbors were capable of editing, whereas those not connected were non-functional, providing further confidence in the network that was constructed. From these V4 was chose, which demonstrated high editing capability while still bearing 7 simultaneous mutations, as well as family members V3, a variant bearing two mutations, and V5, a variant bearing one additional mutation from V4. The efficacy of these mutants was further evaluated side-by-side with wt SpCas9 across a panel of genes and cell types, and assessed V4 activity across both targeted genome editing and epigenome regulation experiments (FIG. 6a-c). Together, these results confirmed that leveraging the unique combinatorial library design and screening strategy, Cas9 variants were produced with multiple top immunogenic epitopes simultaneously mutated (Figure Sc) while still retaining significant genome targeting functionality.

Delivery of wt SpCas9 and SpCas9v2 and CRISPRoff versions of the same as icRNAs was then evaluated. Both robust genome and epigenome targeting via the icRNA delivery format was observed (FIGS. 2F, 2G).

### Example 2: Robust Protein Translation and Genome Targeting Via In Situ Circularization of Large mRNA Payloads

Cell culture: HEK293T and HeLa cells were cultured in DMEM supplemented with 10% FBS and 1% Antibiotic-Antimycotic (Thermo Fisher). K562 cells were cultured in RPMI supplemented with 10% FBS and 1% Antibiotic-Antimycotic (Thermo Fisher). All cells were cultured in an incubator at 37° C. and 5% CO₂.

DNA transfections were performed by seeding HEK293T cells in 12 well plates at 25% confluency and adding 1 μg of each DNA construct and 4 μL of Lipofectamine 2000 (Thermo Fisher). RNA transfections were performed by adding 1 μg of each RNA construct and 3.5 μL of Lipofectamine MessengerMax (ThermoFisher). Electroporations were performed in K562 cells using the SF Cell Line 4D-Nucleofector X Kit S (Lonza) per manufacturer's protocol.

In vitro transcription: DNA templates for generating desired RNA products were created by PCR amplification from plasmids or gBlock gene fragments (IDT) and purified using a PCR purification kit (Qiagen). Plasmids were then generated with these templates containing a T7 promoter followed by 5′ ribozyme sequence, a 5′ ligation sequence, an IRES sequence linked to the product of interest, a 3′ UTR sequence, a 3′ ligation sequence, a 3′ ribozyme sequence, and lastly a poly-T stretch to terminate transcription. Linearized plasmids were used as templates and RNA products were then produced using the HiScribe 17 Quick High Yield RNA Synthesis Kit (NEB E2050) per manufacturer's protocol. For experiments utilizing m6A modified RNA, the Hiscribe T7 High Yield RNA Synthesis Kit (NEB E2040) was used, where 5% of ATP was substituted with N6-Methyladenosine-5′-Triphosphate (Trilink Biotechnologies N-1013). Linear mRNA was produced using the HiScribe T7 mRNA Kit with CleanCap Reagent AG (NEB E2080). All UTP was replaced with N1-Methylpseudouridine-5′-Triphosphate (Trilink Biotechnologies N-1081).

In vitro persistence experiments: To assess persistence of circular icRNA, HEK293T cells were transfected with linear icRNA GFP (which circularizes after delivery to HEK293T cells to produce circular icRNA GFP) or linear icdRNA and RNA was isolated 6 hours, one day, two days, and three days after transfection. qPCR was performed to assess the amount of GFP RNA and RT-PCR was performed to confirm circular icRNA persistence in cells receiving linear icRNA.

Persistence of circular icRNA containing a ECMV IRES, a WPRE, and a 50 adenosine poly(A) stretch compared to commercially sourced RNA (Trilink Biotechnologies, L-7601) with a 5′ cap and a poly(A) tail was similarly performed, with additional time points at day 4 and 5. RNA was isolated from cells and qPCR was performed to assess the amount of GFP RNA. The circular icRNAs are formed upon delivery of a precursor linear icRNAs into cells that circularized the linear icRNAs.

For cardiomyocyte experiments, H1 human embryonic stem cells were differentiated into cardiomyocytes using established protocols (98, 99). Briefly, stem cells were dissociated using Accutase and seeded into 12 well Matrigel coated plates. Cells were maintained in mTeSR1 (StemCell Technologies) for 3-4 days until cells reached about 95% confluence. Media was changed to RPMI containing B27 supplement and 10 μM CHIR99021. After 24 hours, media was changed to RPMI containing B27 supplement without insulin. Two days later, media was changed such that half of the cultured media was mixed with fresh RPMI containing B27 supplement without insulin and 5 μM IWP2. After two days, media was changed to RPMI containing B27 supplement without insulin. Media was then changed to RPMI containing B27 supplement every two days after that. Cardiomyocytes were transfected with linear icRNA containing an EMCV or CVB3 IRES, a WPRE, and a 165 adenosine poly(A) stretch (linear icRNA generates circular icRNA after transfection into the cardiomyocytes) or Trilink mRNA twelve days after CHIR99021 induction. 15 images were taken for each condition at each timepoint and GFP intensity was quantified using FIJI (NIH).

Flow cytometry experiments: To assess in vitro protein translation efficiencies, equal concentration of linear icRNA or Trilink mRNA was transfected into HEK293 Ts and GFP intensity was quantified the next day. GFP intensity, defined as the median intensity of the cell population, was quantified after transfection using a BD LSRFortessa Cell Analyzer.

Quantifying circular efficiency: To assess circular efficiency, linear icRNA containing an ECMV or CVB3 IRES, a WPRE, and a 165 adenosine poly(A) stretch was generated. RNA was then either frozen or pre-circularized using the RTCB ligase (NEB M0458S) per manufacturer's instructions. To remove any linear RNA, pre-circularized RNA was treated with RnaseR (Lucigen RNR07250) per manufacturer's instructions. Linear icRNA or pre-circularized icRNA was then transfected into HEK293 Ts and RNA was isolated from cells at 6, 24, and 48 hours. RT-PCR was performed and the intensity of the band for circular icRNAs generated from the circularization of linear icRNAs transfected into HEK293 Ts compared to pre-circularized icRNA was defined as the circular efficiency. All circular intensity values were normalized to respective GAPDH band intensity.

Lipid nanoparticle formulations: (6Z,9Z,28Z,31Z)-heptatriaconta-6,9,28,31-tetraen-19-yl-4-(dimethylamino) butanoate (DLin-MC3-DMA) was purchased from BioFine International Inc. 1,2-distearoyl-sn-glycero-3-phosphocholine (DSPC) and 1,2-dimyristoyl-rac-glycero-3-methoxypolyethylene glycol-2000 (DMG-PEG-2000) were purchased from Avanti Polar Lipids. Cholesterol was purchased from Sigma-Aldrich. mRNA LNPs were formulated with DLin-MC3-DMA:cholesterol:DSPC:DMG-PEG at a mole ratio of 50:38.5:10:1.5 and a N/P ratio of 5.4. To prepare LNPs, lipids in ethanol and mRNA in 25 mM acetate buffer, pH 4.0 were combined at a flow rate of 1:3 in a PDMS staggered herringbone mixer (100, 101). The dimensions of the mixer channels were 200 by 100 um, with herringbone structures 30 um high and 50 um wide. Immediately after formulation, 3 volumes of PBS was added and LNPs were purified in 100 kDa MWCO centrifugal filters by exchanging the volume 3 times. Final formulations were passed through a 0.2 um filter. LNPs were stored at 4° C. for up to 4 days before use. LNP hydrodynamic diameter and polydispersity index were measured by dynamic light scattering (Malvern NanoZS Zetasizer). The mRNA content and percent encapsulation were measured with a Quant-iT RiboGreen RNA Assay (Invitrogen) with and without Triton X-100 according to the manufacturer's protocol.

Animal experiments: All animal procedures were performed in accordance with protocols approved by the Institutional Animal Care and Use Committee of the University of California, San Diego. All mice were acquired from Jackson Labs.

To confirm circularization of linear icRNA constructs in vivo, 10 μg of linear GFP icRNA or linear GFP icdRNA LNPs were injected retro-orbitally into C57BL/J6J mice. After 3 days and 7 days, livers were isolated and placed in RNAlater (Sigma-Aldrich). RNA was later isolated using QIAzol Lysis Reagent and purified using RNeasy Mini Kit (Qiagen) according to the manufacturer's protocol. Amount of circularized RNA were assessed by RT-qPCR.

To assess circular icRNA persistence in vivo, equal concentration of linear EPO icRNA containing an ECMV or CVB3 IRES, WPRE, and a 165 adenosine poly(A) stretch (15 μg LNPs for EMCV) or linear EPO RNA were injected retro-orbitally into C57BL/6J mice. Blood draws were performed on days 0, 1, 2, 3, 4, and 7, serum was separated using blood collection tubes (Sarstedt), and EPO expression was then assessed by a sandwich enzyme-linked immunosorbent assay (ELISA, R&D Systems DEP00). EPO ELISA was performed per manufacturer's instructions, with 8 μL of serum used for each mouse. Persistence was assessed by dividing the EPO expression over time to the respective day 1 expression. On day 7, livers were isolated and RNA was extracted. qPCR was performed to assess EPO mRNA expression amongst the conditions and RT-PCR was performed to ensure circularization of the injected linear icRNA.

Cas9 alignment and mutation selection: Naturally occurring variation in Cas9 sequence space was explored by aligning BLAST hits of the SpCas9 amino acid sequence. This set was then pruned by removing truncated, duplicated, or engineered sequences, and those sequences whose origin could not be determined. At specified immunogenic epitopes and key anchor residues, top alternative amino acids were obtained using frequency in the alignment weighted by overall sequence identity to the wild type SpCas9 sequence, such that commonly occurring amino acid substitutions appearing in sequences highly similar to the wild-type were prioritized for further analysis and potential inclusion in the LORAX library.

HLA freguencv estimation and binding predictions: HLA-binding predictions were carried out using netMHC4.1 or netMHCpan3.1. Global HLA allele frequencies were estimated from data at allelefrequencies.net as follows. Data was divided into 11 geographical regions. Allele frequencies for each of those regions were estimated from all available data from populations therein. These regional frequencies were then averaged weighted by global population contribution. Alleles with greater than 0.001% frequency in the global population, or those with greater than 0.01% in any region, were included for further analysis and predictions.

Immunogenicity scores: The vector of predicted nM affinities output by netMHC were first normalized across alleles to account for the fact that some alleles have higher affinity across all peptides, and to allow for the relatively equivalent contribution of all alleles. These values were then transformed using the 1-log(affinity) transformation also borrowed from netMHC such that lower nM affinities will result in larger resulting values. These transformed, normalized affinities are then weighted by population allele frequency and summed across all alleles and epitopes. Finally, the scores are standardized across proteins to facilitate comparison.

Identification of HPRT1 Guide: The lentiCRISPR-v2 plasmid (Addgene #52961) was first digested with Esp3I and a guide targeting the HPRT1 gene was cloned in via Gibson assembly. After lentivirus production, HeLa cells were seeded at 25% confluency in 96 well plates and transduced with virus (lentiCRISPR-v2 with or without HPRT1 guide) and 8 μg/mL polybrene (Millipore). Virus was removed the next day and 2.5 μg/mL puromycin was added to remove cells that did not receive virus two days later. After 2 days of puromycin selection, 0-14 μg/mL 6-TG was added. After 5 days, cells were stained with crystal violet, solubilized using 1% sodium dodecyl sulfate, and absorbance was measured at 595 nm on a plate reader. 6 μg/mL was chosen due to the lack of cells in the negative control.

Generation of variant Cas9 library: Cas9 variant sequences were generated by separating the full-length gene sequence into small sections, where each section contained wildtype or variant Cas9 sequences. Degenerate pools of these gBlocks were PCR amplified and annealed together, yielding a final library size of 1,492,992 elements. The lentiCRISPR-v2 plasmid containing the HPRT1 guide was digested with BamHI and XbaI and Gibson assembly was used to clone elements into the vector. The Gibson reactions were then transformed into electrocompetent cells and cultured at 37° C. overnight. Plasmid DNA was isolated using the Qiagen Plasmid Maxi Kit and library coverage was estimated by calculating the number of colonies found on LB-carbenicillin plates. DNA was then used to create lentivirus containing the variant Cas9 library.

Cas9 Screen: HeLa cells were seeded in 15 15-cm plates at a density of 10 million cells/plate and transduced with virus containing the variant Cas9 library and 8 μg/mL polybrene the next day at a MOI of 0.3. Media was changed the next day and 2.5 μg/mL puromycin was added to remove cells that did not receive virus two days later. 6 μg/mL 6-TG was added to media once cells reached 90% confluency. Media was changed every other day for ten days to allow for selection of cells containing functional Cas9 variants. After ten days, cells were lifted from the plates and DNA was isolated using the DNeasy Blood & Tissue Kit per manufacturer's protocol.

Nanopore Sequencing: Pre-screen analysis of the Cas9 variant library elements was performed by amplifying the sequence from the plasmid. 1 μg of the variant Cas9 sequences was used for library preparation using the Ligation Sequencing Kit (Oxford Nanopore Technologies, SQK-LSK109) per manufacturer's instructions. DNA was then loaded into a MinION flow cell (Oxford Nanopore Technologies, R9.4.1). Post-screen analysis of library elements was performed by amplifying the Cas9 sequences from 75 μg of genomic DNA. 1 μg of the variant Cas9 sequences was similarly prepared using the Ligation Sequencing Kit and sequenced on a MinION flow cell.

Base calling and genotyping: Raw reads coming off the MinION flow cell were base-called using Guppy 3.6.0 and aligned to an SpCas9 reference sequence containing non-informative NNN bases at library mutation positions, so as not to bias calling towards wild-type or mutant library members, using Minimap2's map-ont presets. Reads covering the full length of the Cas9 gene with high mapping quality were genotyped at each individual mutation site and tabulated to the corresponding library member. Reads with ambiguous sites were excluded from further analysis.

Cluster analysis: Network analysis was performed by first thresholding genotypes to include only those identified as hits from the screen. These were genotypes appearing in the pre-screen plasmid library, both post-screen replicates, and having a fold-change enrichment larger than the wild-type sequence (4.5-fold enrichment). These hits were used to create a graph with nodes corresponding to genotypes and node sizes corresponding to fold change enrichment. Edges were placed between nodes at most 4 mutations distant from each other, and edge weights were defined by 1/d where d is distance between genotypes. Network analysis was done using python bindings of igraph. Plots were generated using the Fruchterman-Reingold force-directed layout algorithm.

HDR validation: Lentivirus was produced from a plasmid containing a GFP sequence with a stop codon and 68 bp AAVS1 fragment. HEK293T cells were treated with 8 μg/mL polybrene and lentivirus. After puromycin selection to create a stable line, cells were transfected with plasmids containing variant Cas9 sequences, a guide targeting the AAVS locus and a GFP repair donor plasmid. After 3 days, FACS was performed and percent GFP positive cells were quantified.

Genome engineering experiments: To validate variant Cas9 functional cutting, variant Cas9 and guides were transfected into HEK293T cells. After two days, genomic DNA was isolated. Genomic DNA was also isolated after two days from K562 cells after electroporation. To assess activity of CCR5 ZFNs delivered as icRNAs, HEK293 Ts were transfected with linear icRNA (which undergoes circularization in the cells to form the corresponding circular icRNA) or linear icdRNA and genomic DNA was isolated after three days. Assessment of GFP ZFN was performed by transfecting HEK293 Ts stably expressing a broken GFP with linear icRNA or linear icdRNA and isolating genomic DNA after three days. To assess activity of Cas9 delivered as icRNAs, HEK293 Ts and K562 were transfected or nucleofected with Cas9 WT or Cas9 v4 along with a guide RNA (synthesized via Synthego) and genomic DNA was isolated after three days.

Zinc finger experiments were performed by transfecting HEK293T cells with 0.5 μg of left and right arms of each zinc finger as either linear icRNA (which undergoes circularization in the cells to form the corresponding circular icRNA) or icdRNA. After three days, genomic DNA was isolated.

Epigenome engineering experiments: dCas9-VPR experiments were performed by transfecting HEK293T cells with dCas9 wt-VPR or dCas9v4-VPR with or without a gRNA targeting the ASCL1 gene. Likewise, KRAB-dCas9 experiments were performed by transfecting cells with KRAB-dCas9 wt or KRAB-dCas9v4 with or without a gRNA targeting the CXCR4 gene. CRISPRoff experiments were performed by transfecting HEK293T cells with circular icRNA CRISPRoffwt or CRISPRoffv4 with or without a gRNA targeting the B2M gene (Synthego). RNA was isolated three days later and repression or activation of genes was assessed by qPCR

Quantification of editing using NGS: After extraction of genomic DNA, PCR was performed to amplify the target site. Amplicons were then indexed using the NEBNext Multiplex Oligos for Illumina kit (NEB). Amplicons were then pooled and sequenced using a Miseq Nano with paired end 150 bp reads. Editing efficiency was quantified using CRISPResso2.

Cas9 Specificity: RNA isolated from the CRISPRoff experiment was used to assess specificity. RNAseq libraries were generated from 300 ng of RNA using the NEBNext Poly(A) mRNA magnetic isolation module and NEBNext Ultra II Directional RNA Library Prep kit for Illumina and sequenced on the Illumina NovaSeq 6000 with paired end 100 bp reads. Fastq files were mapped to the reference human genome hg38 using STAR aligner. Differential gene expression was analyzed using the Bioconductor package DESeq2 with the cutoff of log 2(fold change) greater than 0.5 or less than −0.5 and a p-value less than 10-3. To identify differentially expressed genes, CRISPRoff WT and V4 samples containing the B2M guide were compared to samples not receiving the guide.

ELISpetaay: TAP-deficient T2 cells were a generous gift from Stephen Schoenberger lab. PBMCs were purchased from StemCell Technologies. All donors contained the HLA-A*0201 allele. Both cell lines were maintained in RPMI1640 media supplemented with 10% FBS, 1% Penicillin-Streptomycin, 10 mM HEPES, and 1 mM sodium pyruvate. On the first day, PBMCs were thawed and rested overnight at a density of 106 cells/mL. T2 cells were pulsed with peptides at 10 μg/mL overnight. Peptides were produced from Genscript's Custom Peptide Synthesis service at crude purity. Lastly, 96-well plates (Immobilon-P, Millipore) were coated with 10 μg/mL anti-IFNγ monoclonal antibody (1-DIK, Mabtech) overnight at 4° C. The next day, T2 cells were washed two times and 50,000 T2 cells and 100,000 PBMCs were added to each well. 4 replicates were used per condition. After 22 hours, cells were removed from the plate and 2 μg/mL biotinylated anti-IFNγ secondary antibody (7-B6-1, Mabtech) was added for 2 hours. Plates were washed and 1:1000 Streptavidin-ALP (3310-10-1000, Mabtech) was added for 45 minutes. Plates were washed and color was developed by adding BCIP/NBT-plus substrate (3650-10, Mabtech) for 10 minutes. Plates were thoroughly washed in water, dried at room temperature, and spots were automatically counted using an ELISpot plate reader.

To assess the immunogenicity of the full length Cas9 wildtype and variant protein, in vitro transcribed RNA encoding for wildtype or V4 was electroporated into PBMCs as previously described (82, 83). As PBMCs contain both antigen presenting cells (APCs) and T cells, it is possible to electroporate RNA directly into these APCs and assess T cell response via the ELIspot. Electroporation was performed using the P3 Primary Cell 4D-Nucleofector X Kit (Lonza V4XP). Briefly, PBMCs were first thawed and rested overnight at a density of 10⁶ cells/mL. The next day, 1×10⁶ PBMCs were resuspended in 20 μl of Lonza P3 nucleofector solution and mixed with 1 μg RNA. After electroporation, 2×10⁵ cells were added to each well of an ELISpot plate already coated with anti-IFNγ monoclonal antibody as described above. After 28 hours, cells were removed from the plate and the ELISpot assay and analysis was performed as described.

Lentivirus production: HEK293FT cells were seeded in 1 15-cm plate and transfected with 36 μL Lipofectamine 2000, 3 μg pMD2.G (Addgene #12259), 12 μg pCMV delta R8.2 (Addgene #12263), and 9 μg of the lentiCRISPR-v2 plasmid. Supernatant containing viral particles was harvested after 48 and 72 hours, filtered with 0.45 μm Steriflip filters (Millipore), concentrated to a final volume of 1 mL using an Amicon Ultra-15 centrifugal filter unit with a 100,000 NMWL cutoff (Millipore), and frozen at −80° C.

RT-qPCR: cDNA was synthesized from RNA using the Protoscript II First Strand cDNA Synthesis Kit (NEB). qPCR was performed using a CFX Connect Real Time PCR Detection System (Bio-Rad). All samples were run in triplicates and results were normalized against GAPDH expression. Primers for qPCR are listed in Table 3 below.

TABLE 1
IRES Constructs Screened

| No. | Name | SEQ ID NO: |
|---|---|---|
| 1 | IRES_Cricket Paralysis Virus | 1361 |
| 2 | IRES_Homo Sapiens IGF2 | 1362 |
| 3 | IRES_Hepatovirus A | 1363 |
| 4 | IRES_Hepatitis C Virus H77 Isolate | 1364 |
| 5 | IRES_Homo Sapiens FGF1 | 1365 |
| 6 | IRES_Bovine Viral Diarrhea Virus 1 | 1366 |
| 7 | IRES_Human Rhinovirus A89 | 1367 |
| 8 | IRES_Pan Paniscus LIMA1 | 1368 |
| 9 | IRES_Human Adenovirus 2 | 1369 |
| 10 | IRES Montana Myotis | 1370 |
| | Leukoencephelitis Virus | |
| 11 | IRES Homo Sapiens RANBP3 | 1371 |
| 12 | IRES_Pestivirus Giraffe 1 | 1372 |
| 13 | IRES_Homo Sapiens TGIF1 | 1373 |
| 14 | IRES_Human Poliovirus 1 Mahoney | 1374 |
| 15 | IRES Foot-and-Mouth Disease Virus | 1375 |
| | Type O | |
| 16 | IRES Encephalomyocarditis Virus_7A | 1376 |
| 17 | IRES Encephalomyocarditis Virus_6A | 1377 |
| 18 | IRES Enterovirus 71 | 1378 |
| 19 | IRES_Coxsackievirus B3 | 1379 |

TABLE 2
3′ UTR Constructs Screened

| No. | Name | SEQ ID NO: |
|---|---|---|
| 1 | IRES_Encephalomyocarditis Virus_7A | 1376 |
| 2 | IRES_Encephalomyocarditis Virus_6A | 1377 |
| 3 | IRES_Encephalomyocarditis Virus_6A + WPRE | 1380 |
| 4 | IRES_Encephalomyocarditis Virus_6A + WPRE + pA50 3′ | 1381 |
| 5 | IRES_Encephalomyocarditis Virus_6A + WPRE + pA120 3′ | 1382 |
| 6 | IRES_Encephalomyocarditis Virus_6A + WPRE + pA165 3′ | 1383 |
| 7 | IRES_Encephalomyocarditis Virus_6A + WPRE + pA165 3′ + mtRNR1-AES | 1384 |
| | 3′ UTR | |
| 8 | IRES_Encephalomyocarditis Virus_6A + WPRE + pA165 3′ + mtRNR1- | 1385 |
| | LSP1 3′ UTR | |
| 9 | IRES_Encephalomyocarditis Virus_6A + WPRE + pA165 3′ + AES-mtRNR1 | 1386 |
| | 3′ UTR | |
| 10 | IRES_Encephalomyocarditis Virus_6A + WPRE + pA165 3′ + AES-hBg 3′ | 1387 |
| | UTR | |
| 11 | IRES_Encephalomyocarditis Virus_6A + WPRE + pA165 3′ + FCGRT-hBg 3′ | 1388 |
| | UTR | |
| 12 | IRES_Encephalomyocarditis Virus_6A + WPRE + pA165 3′ + 2hBg 3′ UTR | 1389 |
| 13 | IRES_Encephalomyocarditis Virus_6A + WPRE + pA165 3′ + HBA1 3′ UTR | 1390 |
| 14 | IRES_Coxsackievirus B3 + WPRE + pA165 3′ | 1391 |
| 15 | icChang_AES-mtRNR1 | 1392 |
| 16 | icChang_HBA1 | 1393 |
| 17 | Trilink Biotechnologies commercial linear mRNA | — |

TABLE 3
qPCR Primers

| | | SEQ |
|---|---|---|
| | | ID |
| Name | Sequence | NO: |
| CXCR4_F | GAAGCTGTTGGCTGAAAAGG | 1394 |
| CXCR4_R | CTCACTGACGTTGGCAAAGA | 1395 |
| ASCL1_F | CGCGGCCAACAAGAAGATG | 1396 |
| ASCL1_R | CGACGAGTAGGATGAGACCG | 1397 |
| B2M_F | TATGCCTGCCGTGTGAACCATGT | 1398 |
| B2M_R | GGCATCTTCAAACCTCCATGATGCT | 1399 |
| hGAP_F | ACAGTCAGCCGCATCTTCTT | 1400 |
| hGAP_R | ACGACCAAATCCGTTGACTC | 1401 |
| hPCSK9_F | ATCCACGCTTCCTGCTGC | 1402 |
| hPCSK9_R | CACGGTCACCTGCTCCTG | 1403 |
| GFP_F | ACGTAAACGGCCACAAGTTC | 1404 |
| GFP_R | AAGTCGTGCTGCTTCATGTG | 1405 |
| circ_F | ACAACCACTACCTGAGCACC | 1406 |
| circ_R | AATTCCGCGGGCCCGT | 1407 |

RNAs have emerged as a powerful therapeutic class. However their typically short half-life impacts their activity both as an interacting moiety (such as siRNA), as well as a template (such as mRNAs). Towards this, RNA stability has been modulated using a host of approaches, including engineering untranslated regions, incorporating cap analogs, modifying nucleosides, and optimizing codons (1-5). More recently, novel circularization strategies, which remove free ends necessary for exonuclease-mediated degradation thereby rendering RNAs resistant to most mechanisms of turnover, have emerged as a particularly promising methodology (6-15). However, simple and scalable approaches to achieve efficient production and purification of circular RNAs are lacking, thus limiting their broader application in research and translational settings.

Circular guide RNAs for programmable RNA editing was engineered. The primary approach was via delivery of encoding DNA molecules where the guide RNAs were expressed using pol-III promoters, and thereby were both generated and circularized in cells. However, it was observed that in vitro transcribed RNAs delivered in linear form could successfully circularize in situ in cells upon entry and were similarly functional as guide RNAs. Motivated by the extreme simplicity of this latter approach, and its compatibility with routine in vitro synthesis and purification processes, this framework was explored to determine if it could also be used to generate circular RNAs encoding messenger RNAs. Indeed, as demonstrated herein engineered in situ gircularized RNAs (icRNAs) enable extensive protein translation, and demonstrate their utility across both in vitro and in vivo settings, and across persistent transgene delivery and genome targeting applications.

Common to all these applications enabled via linear icRNA delivery is the critical consideration of their immune system interactions. Although for some applications, such as vaccines, robust immune responses to a delivered transgene are desirable, for other applications such as genome and epigenome targeting, immune responses can instead inhibit therapeutic effects (18, 19). Inducing immune responses through RNA delivery has been researched in vaccine development and proven through the success of COVID vaccines based on this technology (20-23). However, despite substantial engineering efforts, deimmunization remains a problem (24). Thus, to facilitate compatibility between persistence of expression and immunogenicity especially when delivering non-human payloads via icRNAs, a long-range multiplexed (LORAX) protein engineering methodology was developed based on high-throughput screening of combinatorially deimmunized protein variants. This was applied to identify a Cas9 variant with seven key HLA-restricted epitopes simultaneously immunosilenced after a single round of screening, and showed that icRNA-mediated delivery of the same enabled robust genome targeting.

Results: To engineer linear icRNAs, in vitro transcribed linear RNAs were generated that bear a twister ribozyme (also referred to as linearized ribozyme-RNAs) flanked internal ribosome entry site (IRES) coupled to a messenger RNA of interest and a 3′ untranslated region (UTR) (FIG. 7A). Once transcribed, the flanking twister ribozymes rapidly self-cleave, enabling hybridization of the complementary ligation stems to one another. Upon delivery into cells, these linear RNAs (linear icRNA; also referred to as linearized ribozyme activated RNAs) are then circularized in situ via ligation of the proximal 5′ and 3′ ends by the ubiquitous cellular RNA ligase RtcB to generate circular icRNAs. To evaluate this circularization approach, green fluorescent protein (GFP) RNA persistence was assayed in the circular icRNA format in vitro in HEK293T cells. As a direct side-by-side comparison, a linear in situ Circularization gefective RNAs (icdRNAs) was engineered by utilizing catalytically inactive mutants of the twister ribozymes. Specifically, HEK293 Ts were transfected with linear GFP icRNA or linear icdRNA, wherein the linear GFP icRNA circularized in the HEK293 Ts to generate circular GFP icRNA, and RNA was isolated at 6 hours, 1 day, 2 days and 3 days after transfection. Similar amounts of GFP RNA was observed at 6 hours (FIG. 7B, left panel), confirming that approximately equal quantities of icRNA and icdRNA were delivered to cells. However, GFP RNA with functional circularization was significantly higher at days 1, 2, and 3 than icdRNA, indicating improved RNA persistence via circularization (FIG. 7B, middle panel). This improved RNA persistence also correlated with increased GFP translation at 3 days (FIG. 7B, right panel). To confirm linear GFP icRNAs were covalently circularized in cells upon delivery in vitro, RT-PCR was performed by designing outward facing primers that selectively amplified only the circularized RNA molecules. Indeed, only a PCR product for circular GFP icRNAs was observed, confirming successful circularization (FIG. 7C).

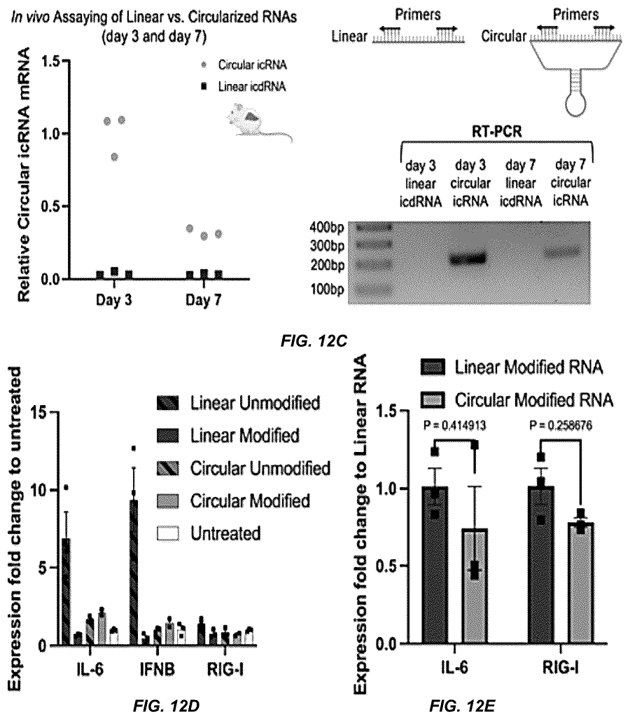

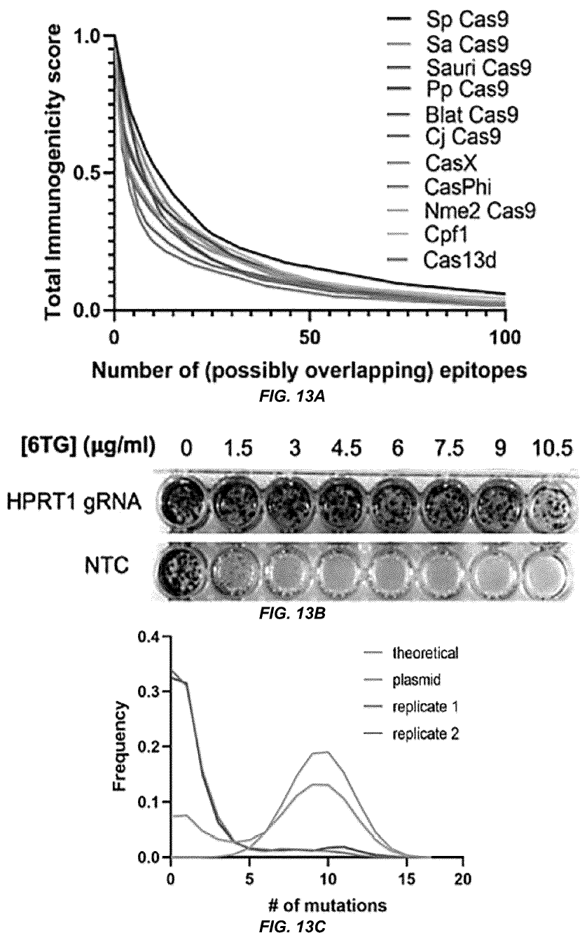

To improve protein translation from icRNAs, a panel of 19 naturally occurring and synthetic IRES sequences were screened (Table 1) (27-231). The 6A form of the Encephalomyocarditis virus IRES (EMCV, FIG. 7D, blue bar, #17), the Enterovirus 71 IRES, and the Coxsackievirus B3 IRES (CVB3, FIG. 7D, green bar, #19) were found to be the best at enabling cap-independent protein translation. To further improve protein translation, a panel of 3′ untranslated regions (UTRs, Table 2) were screened in the context of the EMCV 6A IRES. Addition of a Woodchuck Hepatitis Virus Posttranscriptional Regulatory Element (WPRE) and a poly(A) stretch improved relative protein translation by over 5 fold, but additional 3′ untranslated regions (UTRs) did not improve translation efficiency (FIG. 7E, blue bars, #3-13). For all subsequent studies, icRNAs based on either the EMCV IRES or CVB3 IRES coupled to a WPRE and 165 bp poly(A) stretch were used (FIG. 7E, green bar, #14). Notably, these designs showed enhanced activity (FIG. 7E, gray bars, #15-16). The constructs having 5′ capped linear RNA (FIG. 7E, red bar, #17) enabled higher instantaneous protein translation 24 hrs post delivery (2-3 fold), but the circular icRNAs enabled significantly improved persistence of protein translation over multiple days (FIG. 12A; see, e.g., circle). Next, to assess in situ circularization efficiency, HEK cells were transfected with linear icRNAs or with icRNAs pre-circularized in vitro using RtcB ligation in a test tube followed by RNaseR treatment to enrich pre-circularized icRNA species. RT-PCR was performed using outward facing primers and normalized to GAPDH expression. Across independent icRNA designs (comprising either EMCV IRES (left bar at each time point) or CVB3 IRES (right bar at each time point), efficient (˜20%) in situ circularization levels for icRNAs compared to pre-circularized icRNA was observed (FIG. 7F). As a final check of circularization, the ligation junction was mapped correctly to its predicted location via Sanger sequencing of the RT-PCR product (FIG. 7G). To ensure that linear icRNAs were also capable of circularization in vivo, lipid nanoparticles (LNPs) containing either linear icRNA (that undergoes in vivo circularization to form circular icRNA) or linear icdRNA were generated and retro-orbitally injected them into mice. LNPs for each condition were successfully generated of similar size (FIG. 12B). Livers were isolated three and seven days after injection and RT-PCR confirmed circularization in vivo persisting for at least seven days (FIG. 12C). In addition, linear icRNA and linear icdRNA versions were generated containing 5% N6-methyladenosine (m6A) modification. Compared to unmodified linear RNA which induces a huge innate immune response, the immune responses to N¹-Methylpseudouridine-5′-Triphosphate (pseudo-UTP) modified linear RNA and m6A modified and unmodified linear icRNAs were observed to be low in vitro (FIG. 12D). Taken together, these studies demonstrated the ability to tune protein translation from icRNAs over an order of magnitude via choice of IRES and 3′ UTR sequences.

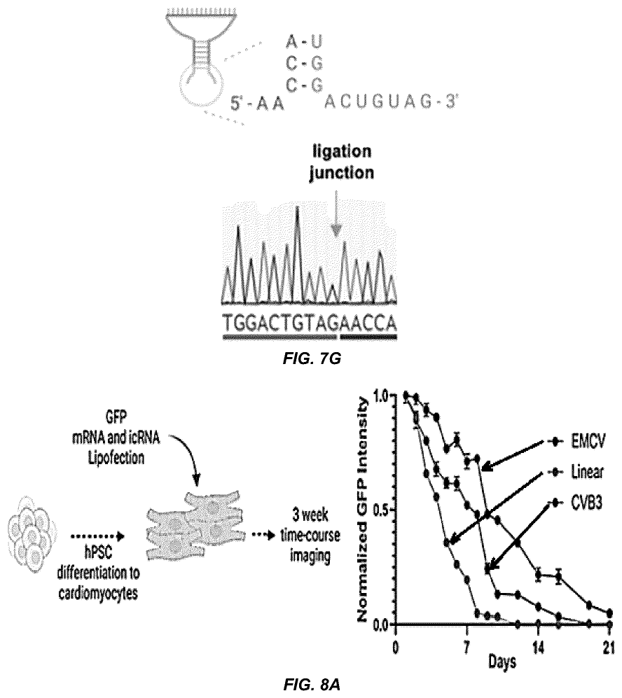

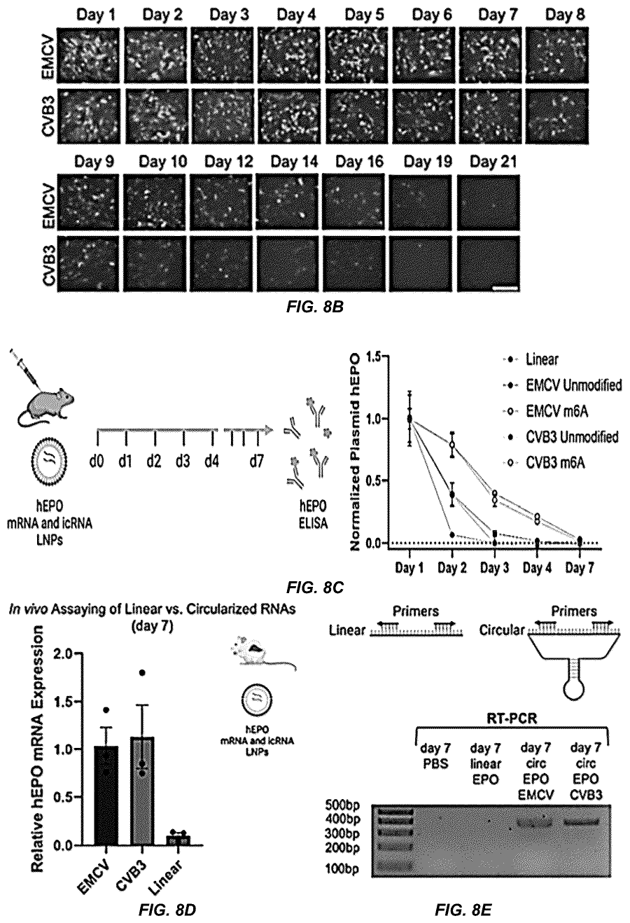

Experiments were performed to validate circular icRNA persistence across in vitro and in vivo settings. First investigation was performed regarding utility in stem cell derived cells. RNA-based strategies have gained increasing relevance in the context of tissue engineering and regenerative medicine, but the short half-life of mRNA severely limits their utility in this space. For example, whereas cell fate reprogramming can take 1-4 weeks to induce, previous studies have demonstrated that protein expression lasts about 4 days after RNA transfection in this context (33, 34). In addition, it is important to note that gene dosage can mediate cell response. For instance, CRISPRa-mediated overexpression of OCT4 induced iPSC reprogramming whereas cDNA-mediated OCT4 overexpression did not due to much higher RNA and protein expression in cDNA-treated conditions (35). Thus, the ideal RNA strategy would have a “Goldilocks” effect: a moderate protein expression that persists for weeks. To that end, linear icRNA containing either an EMCV or CVB3 IRES, a WPRE, and a 165 poly(A) stretch or a commercial linear capped RNA with optimized UTRs were transfected into stem cell-derived cardiomyocytes, wherein the linear icRNA circularized in stem cell-derived cardiomyocytes to generate the corresponding circular icRNA (FIG. 8A; linear icRNA with EMCV IRES is top data line, linear icRNA with CVB3 IRES is middle data line, and commercial linear capped RNA is bottom data line). Very high expression with linear capped RNA was observed with optimized UTRs that, as expected, rapidly declined over 3-5 days, whereas circular icRNA (formed from linear icRNA) exhibited consistent expression that persisted for over two weeks. It was observed that CVB3 IRES (FIG. 7D) induced significantly higher expression in HEK293T cells, interestingly, similar expression levels were observed using the EMCV IRES compared to the CVB3 in the cardiomyocyte context (FIG. 8B), implying potential cellular context dependence on activity of IRESs.

Experiments were then performed to extend these results in vivo. Towards this, lipid nanoparticles (LNPs) (36, 37) were synthesized bearing either linear icRNA or linear RNA encoding for the human erythropoietin (EPO) gene (Figure C). RNA/LNPs were retro-orbitally injected into C57BL/6 mice, wherein the linear icRNA circularized to produce circular icRNA in cells after injection, and serum was collected over one week to assess EPO levels. Interestingly, m6A modification significantly improved circular icRNA persistence compared to linear RNA or unmodified circular icRNA. Consistent with in vitro results, the in vivo innate immune responses to (pseudo-UTP) modified linear RNA and m6A modified icRNAs were also similar (FIG. 12E). Liver transcriptomes were assayed 7 days after injection. While linear EPO RNA was largely degraded by day 7, circular EPO icRNA persisted (FIG. 8D). Furthermore, RT-PCR also confirmed successful in situ circularization of linear icRNA in vivo (Figure E).

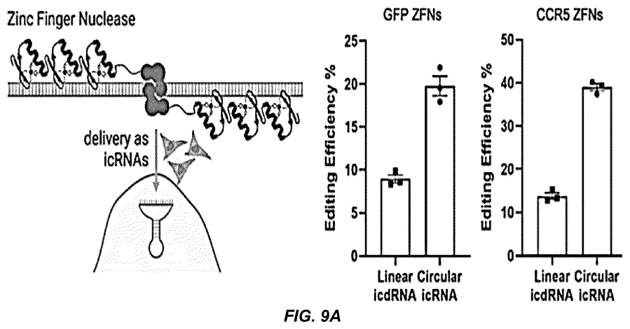

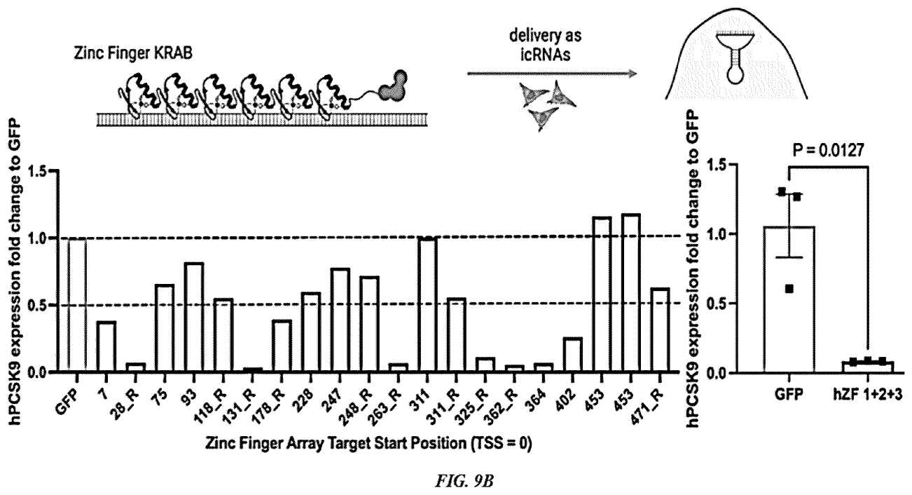

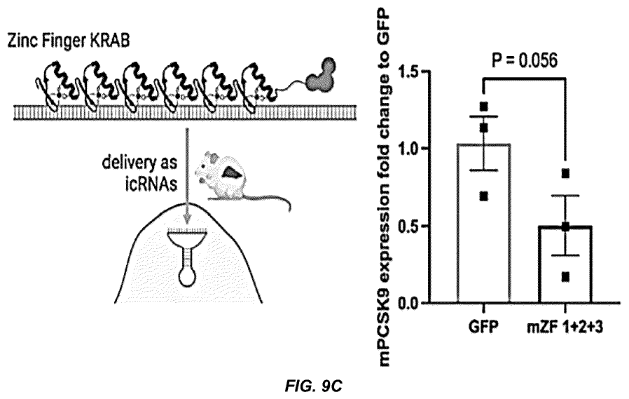

Spurred by the above results, it was hypothesized that this increased persistence of circular icRNAs, in addition to enabling applications entailing sustained transgene expression, could also facilitate efficient genome and especially epigenome targeting. Towards this, icRNA utility was explored in the context of zinc finger (ZF) proteins, as being a solely protein-based genome engineering toolset it was expected ZFs would be particularly suited for this mode of delivery. Indeed a more efficient genome editing via zinc finger nuclease (ZFN) icRNAs was observed compared to corresponding icdRNAs targeting the GFP and CCR5 genes (FIG. 9). Building on this observation, delivery of Zinc Finger KRAB proteins to programmably repress genomic targets was investigated. PCSK9, a gene that encodes an enzyme that regulates low-density lipoprotein (LDL) receptor degradation, was used. Loss-of-function mutations in PCSK9 are associated with reduced risk of cardiovascular disease with no documented adverse side effects (40-42). Antibodies, antisense oligonucleotides, and CRISPRs have all been utilized to target PCSK9 (43-49), and here experiments were performed to examine if a transient pulse of zinc finger epigenome regulators could enable repression of PCSK9. For in vitro studies, HeLa cells comprising a panel of 20 ZF-KRAB proteins delivered as icRNAs were screened (FIG. 9B, left panel). These ZFs tiled across 0-500 bp of the transcription start site (TSS) of hPCSK9, and notably, 9 of these enabled robust gene repression (90-50%). Experiments were then performed to validate 3 of these via concurrent delivery to enable over 95% repression of hPCSK9 in cultured cells (FIG. 9B, right panel). Following this, 10 ZF-KRAB proteins targeting mPCSK9 were screened in mice, delivered as icRNAs packaged in LNPs. Similar to above, the top 3 ZF-KRABs were validated via concurrent delivery as icRNAs loaded into LNPs, observing ˜50% in vivo mPCSK9 repression in mice livers (FIG. 9B). Taken together, these results establish that a transient pulse of zinc finger epigenome regulators delivered as icRNAs can enable robust gene repression both in vitro and in vivo, enabling a genomically scarless and safe approach for modulating therapeutic gene expression.

Building on these results with zinc finger proteins, experiments were performed to explore if circular icRNA persistence could be similarly enhance the activity of CRISPR-Cas9 systems. However, unlike for ZFs, which are built on a human protein chassis, this feature of persistence could aggravate immune responses in therapeutic settings for CRISPR systems as those are derived from prokaryotes, including some residing in the human gut microbiome (50-53). Thus, to enable compatibility between persistence of expression and immunogenicity, experiments were performed to develop a methodology to screen progressively deimmunized SpCas9 proteins by combinatorially mutating particularly immunogenic epitopes.

While variant library screening has proven to be an effective approach to protein engineering (55-62), applying it to deimmunization faces three important technical challenges. One, the need to mutate multiple sites simultaneously across the full length of the protein; two, reading out the associated combinatorial mutations scattered across large (>1 kb) regions of the protein via typical short-read sequencing platforms; and three, engineering fully degenerate combinatorial libraries which can very quickly balloon to unmanageable numbers of variants (24, 63). To overcome these challenges several methodological innovations were developed which, taken together, comprise a novel long range multiplexed (LORAX) protein engineering platform capable of screening millions of combinatorial variants simultaneously with mutations spread across the full length of arbitrarily large proteins (FIG. 10).

Towards library design, in order to narrow down the vast mutational space associated with combinatorial libraries, an approach guided by evolution and natural variation was used. As deimmunizing protein engineering seeks to alter the amino acid sequence of a protein without disrupting functionality, it is extremely useful to narrow down mutations to those less likely to result in non-functional variants. To identify these mutants large alignments of Cas9 orthologs were generated from publicly available data to identify low-frequency SNPs that have been observed in natural environments. Such variants are likely to have limited effect on protein function, as highly deleterious alleles would tend to be quickly selected out of natural populations (if Cas9 activity is under purifying selection) and therefore not appear in sequencing data (66). To further subset these candidate mutations, immunogenicity was evaluated in silico using the netMHC epitope prediction software (67, 68), in order to determine to what degree the candidate mutations are likely to result in the deimmunization of the most immunogenic epitopes in which they appear. This is a useful step as many mutations may have little effect on overall immunogenicity (69-71). Screening for decreased peptide-MHC class I binding filters out amino acid substitutions which are likely immune-neutral, substantially increasing the likelihood of functional hits with enough epitope variation to evade immune induction (71, 72).

Next, long-read nanopore sequencing was applied to measure the results of the screens of our combinatorial libraries. This circumvents the limit of short target regions and obviates the need for barcodes altogether by single-molecule sequencing of the entire target gene, enabling library design strategies which can explore any region of the protein in combination with any other region without any complicated cloning procedures required to facilitate barcoding (73). To date, the adoption of nanopore sequencing has been limited by its high error rate, around 95% accuracy per DNA base (74), as compared to established short read techniques which are multiple orders of magnitude more accurate. To address this challenge, the libraries were designed such that each variant that was engineered would have multiple nucleotide changes for each single target amino acid change, effectively increasing the sensitivity of nanopore based readouts with increasing numbers of nucleotide changes per library member. The large majority of amino acid substitutions are amenable to a library design paradigm in which each substitution is encoded by two, rather than one, nucleotide changes, due to the degeneracy of the genetic code and the highly permissive third “wobble” position of codons.

The scale of engineering which would be required to generate an effectively deimmunized Cas9 is not fully understood, as combinatorial deimmunization efforts at the scale of proteins thousands of amino acids long have not yet been possible. Therefore, to roughly estimate these parameters an immunogenicity scoring metric was developed that takes into account all epitopes across a protein and the known diversity of MHC variants in a species weighted by population frequency to generate a single combined score representing the average immunogenicity of a full-length protein as a function of each of its immunogenic epitopes (75). Formally, this score is calculated as:

$${I}_{x} = \frac{{\sum }_{i}^{m} {\sum }_{j}^{n} {w}_{j} ( 1 - log ( {k}_{i j} * j ) )}{y}$$

Iₓ=Immunogenicity score of protein x, i=epitopes, j=HLA alleles, j=allele specific standardization coefficient, wⱼ=HLA allele weights, kᵢⱼ=predicted binding affinity of epitope i to allele j, and y=protein specific scaling factor.

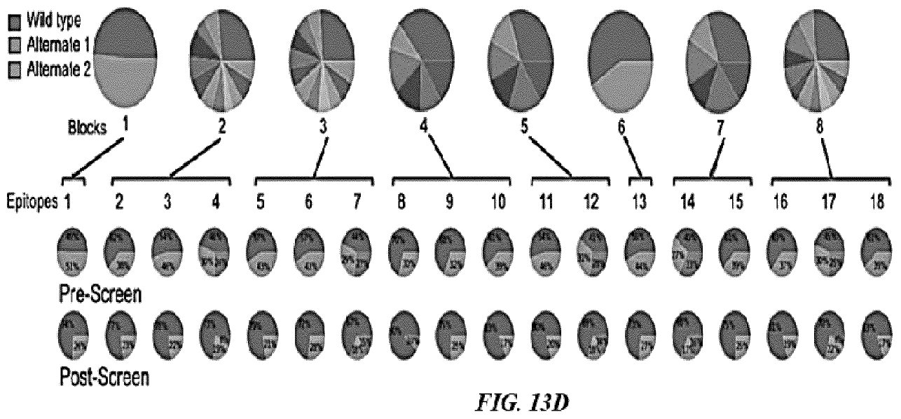

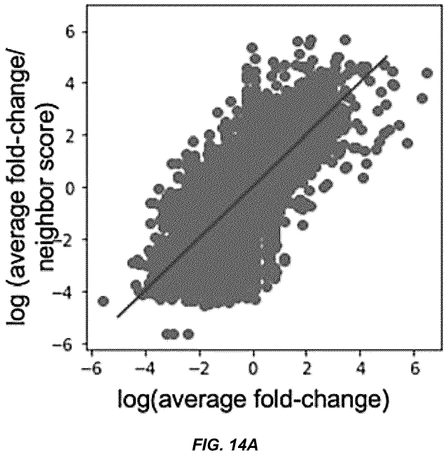

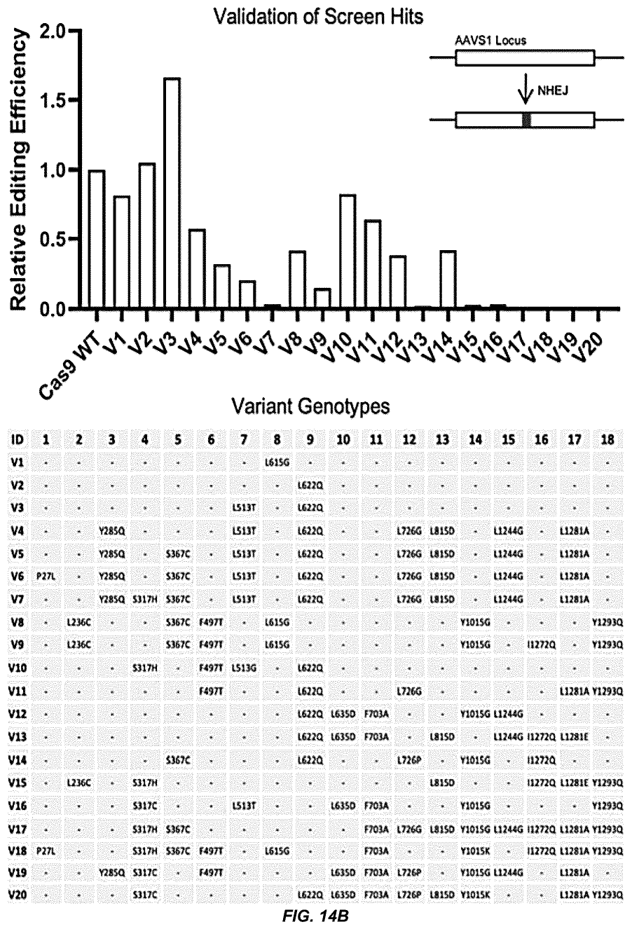

The overall effect of mutating the top epitopes in several Cas9 orthologs was predicted (FIG. 13A). As might be expected, this analysis suggests that single-epitope strategies are woefully inadequate to deimmunize a whole protein for multiple HLA types, and also that there are diminishing returns as more and more epitopes are deimmunized. The analysis suggests that it may require on the order of tens of deimmunized epitopes to make a significant impact on overall, population-wide protein immunogenicity. The scale of engineering demanded by these immunological facts has previously been intractable, but by applying LORAX it is conjectured one could now make substantial steps, several mutations at a time, through the mutational landscape of the Cas9 protein.

Specifically, applying the procedure above, a library of Cas9 variants was designed based on the SpCas9 backbone containing 23 different mutations across 17 immunogenic epitopes (FIG. 10). Combining these in all possible combinations yields a library of 1,492,992 unique elements. With this design, the library was constructed in a stepwise process. First, the full-length gene was broken up into short blocks of no more than 1000 bp, which overlap by 30 bp on each end. Each block is designed such that it contains no more than 4 target epitopes to mutagenize. With few epitopes per block and few variant mutations per epitope, it becomes feasible to chemically synthesize each combination of mutations for each block. Each of these combinations was then synthesized and mixed at equal ratios to make a degenerate block mix. This was repeated for each of the blocks necessary to complete the full-length protein sequence via fusion PCR

To identify functional variants still capable of editing DNA, a positive selection screen targeting the hypoxanthine phosphoribosyltransferase 1 (HPRT1) gene was designed and carried out (76). In the context of the screen, HPRT1 converts 6-thioguanine (6TG), an analogue of the DNA base guanine, into 6-thioguanine nucleotides that are cytotoxic to cells via incorporation into the DNA during S-phase (77). Thus, only cells containing functional Cas9 variants capable of disrupting the HPRT1 gene can survive in 6TG-containing cell culture media. To first identify the optimal 6TG concentration, HeLa cells were transduced with lentivirus particles containing wild-type Cas9 and either a HPRT1-targeting guide RNA (gRNA) or a non-targeting guide. After selection with puromycin, cells were treated with 6TG concentrations ranging from 0-14 μg/mL for one week. Cells were stained with crystal violet at the end of the experiment and imaged. 6 μg/mL was selected as all cells containing non-targeting guide had died while cells containing the HPRT1 guide remained viable (FIG. 13B).

To perform the screen, replicate populations of HeLa cells were transduced with lentiviral particles containing the variant SpCas9 library along with the HPRT1-targeting gRNA at 0.3 MOI and at greater than 75-fold coverage of the library elements. Cells were selected using puromycin after two days and 6TG was added once cells reached 75% confluency. After two weeks, genomic DNA was extracted from remaining cells and full-length Cas9 amplicons were nanopore sequenced on the Oxford Nanopore (ONT) MinION platform.

MinION sequencing confirmed the majority of the pre-screened library consists of Cas9 sequences with significant numbers of mutations, with most falling into a broad peak between 6 and 14 mutations per sequence, each of which knocking out a key immunogenic epitope (FIG. 13C). Interestingly, the post-screening library was significantly shifted in the mutation density distribution, suggesting that the majority of the library with large (>4) numbers of mutations resulted in non-functional proteins which were unable to survive the screen. Meanwhile, wild-type, single, and double mutants were generally enriched as these proteins proved more likely to retain functionality and pass through the screen (FIG. 13C). Additionally, the two independent replicates of the screen showed strong correlation (R²=0.925) providing further evidence of robustness (FIG. 9). the change in overall frequency of mutations in the pre- and post-screen libraries was also analyzed to see if a pattern of mutation effects could be inferred. Although the wild-type allele was enriched at every site in the post-screen sequences, nearly every site retained a significant fraction of mutated alleles, suggesting that the mutations, at least individually, are fairly well-tolerated and do not disrupt Cas9 functionality (FIG. 13D).

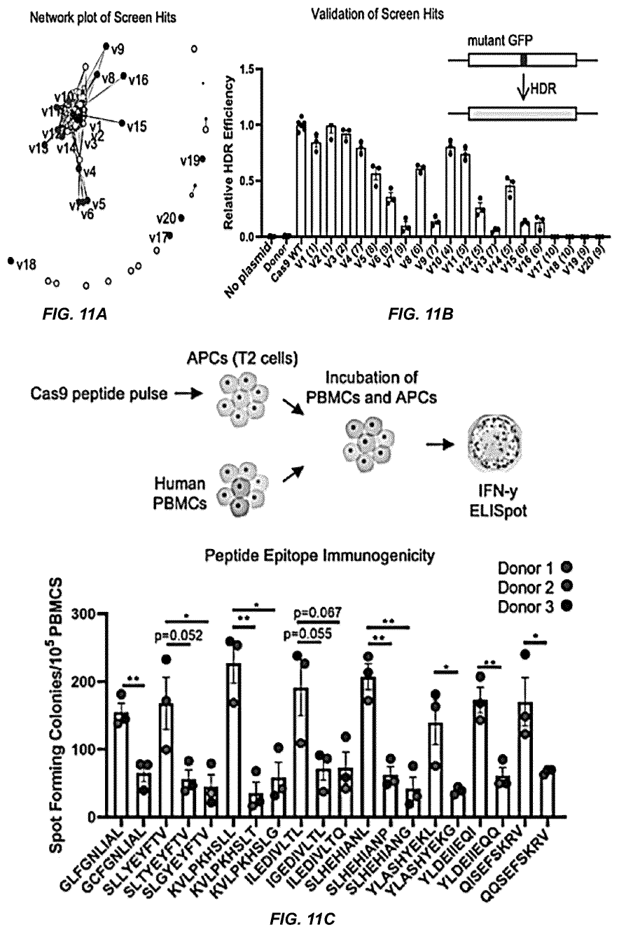

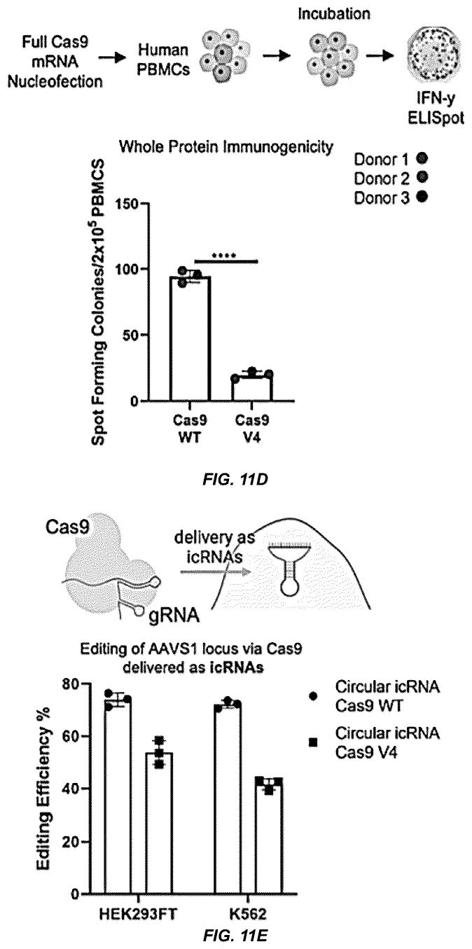

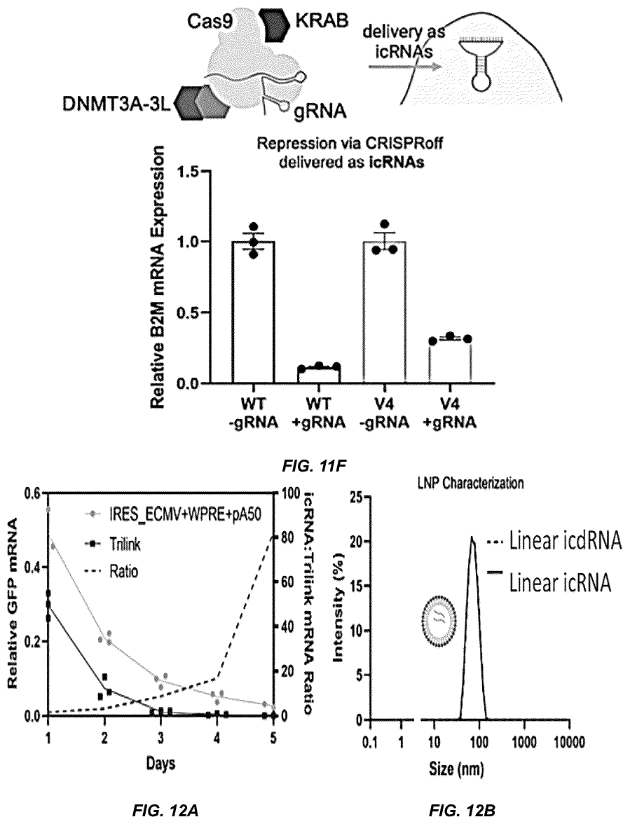

In order to select hits for downstream validation and analysis, a method for differentiating high-support hits likely to be real from noise-driven false positive hits was devised. To do this it was hypothesized that the fitness landscape of the screen mutants is likely to be smooth, i.e. variants that contain similar mutations are more likely to have similar fitnesses in terms of editing efficiency compared to randomly selected pairs (78). This was confirmed by computing a predicted screen score for each variant based on a weighted regression of its nearest neighbors in the screen. This metric correlates well with the actual screen scores and approaches the screen scores even more closely as read coverage increases. This provides good evidence that the fitness landscape is indeed somewhat smooth (FIG. 14A). Next, it was reasoned that because the fitness landscape is smooth, real hits should reside in broad fitness peaks which include many neighbors that also show high screen scores, whereas hits that are less supported by near neighbors are more likely to be spurious as they represent non-smooth fitness peaks. Formalizing this logic, a network analysis was performed to differentiate noise-driven hits from bona fide hits by looking at the degree of connectivity with other hits (FIG. 11A).

Applying these analyses to the screen output led to the selection and construction of 20 variants (V1-20) for validation and characterization. Two independent methods were applied to quantify editing of the deimmunized Cas9 variants. First, a gene-rescue experiment was performed using low frequency homology directed repair (HDR) to repair a genetically encoded broken green fluorescent protein (GFP) gene (79) (FIG. 11B). And second, NHEJ mediated editing was quantified by genomic DNA extraction and Illumina next generation sequencing (NGS) using the CRISPResso2 package (FIG. 14B) (80). Variants highly connected to neighbors were capable of editing, whereas those not connected were non-functional, allowing selection of hits as enriching for truly functional sequences. Among the screen hits was the L614G mutation first identified by Ferdosi and colleagues (14) as a functional Cas9 variant with a critical immunodominant epitope de-immunized (V1). This concordance with previous work provided further confidence in the screening method. Interestingly, another deimmunizing mutation within the same epitope, L622Q (V2) was identified, which similarly retains Cas9 functionality, but appears to be more epistatically permissive, as many of the multi-mutation hits combine this mutation with other deimmunized epitopes. From these multi-mutation hits V4 was chosen, which demonstrated high editing capability while still bearing simultaneous mutations across seven distinct epitopes, as well as family members V3, a variant bearing two mutations, and V5, a variant bearing the seven changes from V4 plus one additional mutation.

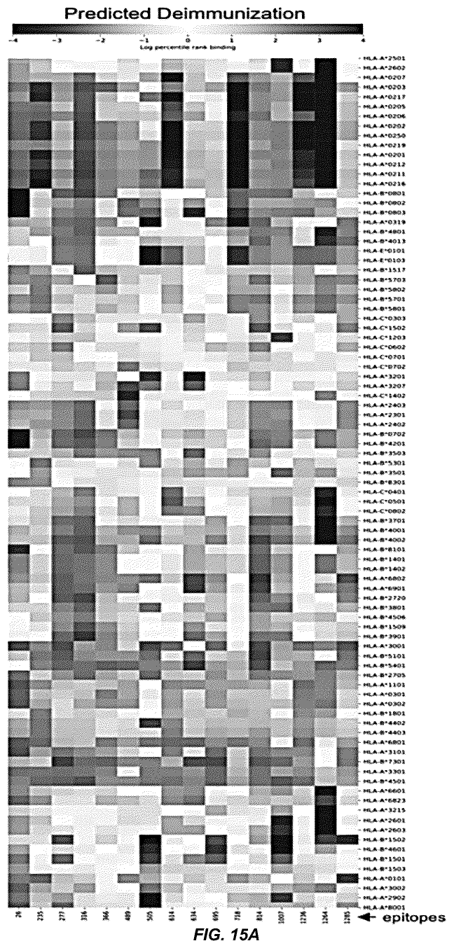

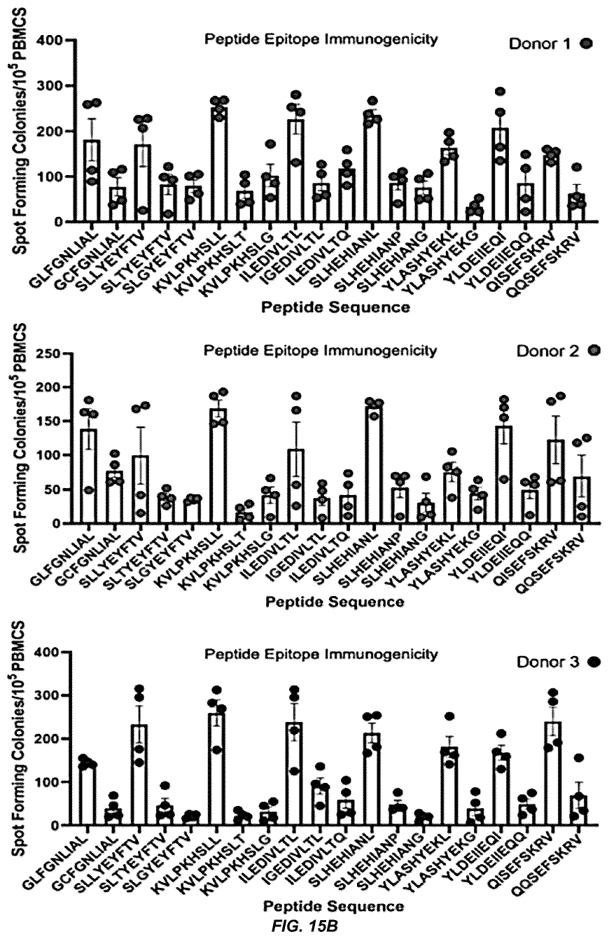

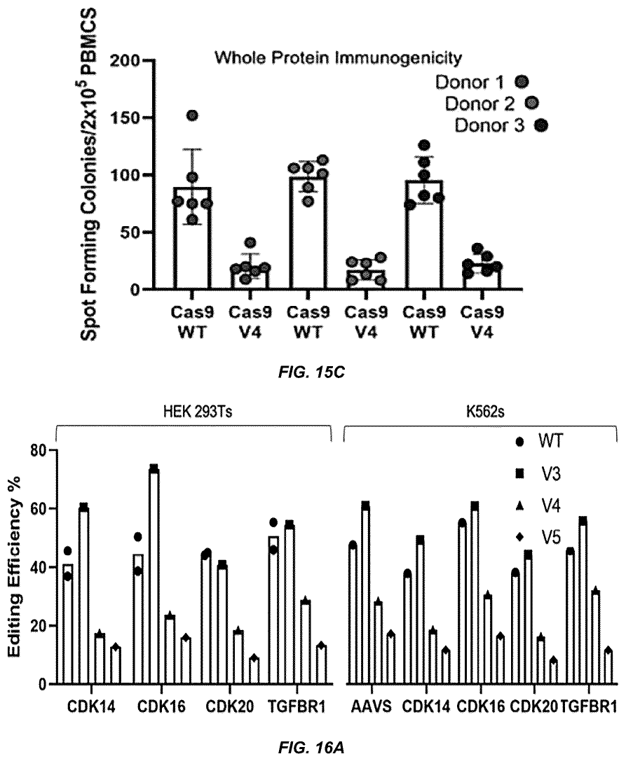

To confirm that mutation of these epitopes indeed elicited de-immunization, T-cell response to wildtype and variant peptides were assessed by measuring IFN-γ secretion in the ELISpot assay (19, 54). Peripheral blood mononuclear cells (PBMCs) were used from three separate donors that carried the HLA-A*0201 allele as peptides were presented to cells using the TAP-deficient cell line T2 (HLA-A*0201 positive) (81). Correspondingly, peptides for epitopes 2, 7, 8, 9, 12, 15, and 16 were synthesized as the predictions suggested these epitopes would induce a reduction in immune response for the HLA-A*0201 allele (FIG. 15A). Importantly, since SpCas9v4 carries four of these mutations, this assay would also provide confirmation of deimmunization for this variant. The data shows that mutant peptides for all epitopes tested indeed resulted in fewer spot forming colonies for all three donors compared to wild type peptides (FIG. 11C, FIG. 15B), thereby confirming the predictions. To assess whether the full length protein is de-immunized, mRNA encoding for SpCas9WT and SpCas9V4 were generated and electroporated this into the PBMC populations. As PBMCs have a mixture of both antigen presenting cells (APCs) and T-cells (82, 83) the ability to introduce the RNA to the APCs and measure T-cell response via the ELISpot assay was possible. Excitingly, significantly fewer spot forming for full length SpCas9v4 compared to SpCas9WT for all three donors was observed (FIG. 11D, FIG. 15C).

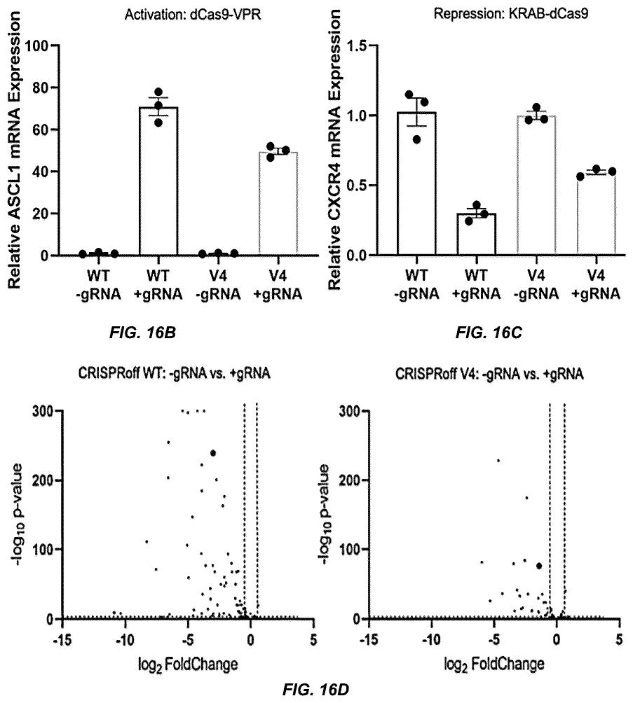

Based on this, the efficacy of these mutants was evaluated side-by-side with WT SpCas9 across a panel of genes and cell types, and assessed V4 activity across both targeted genome editing and epigenome regulation experiments (FIG. 15A-15C) (84). Together, these results confirmed that leveraging the unique combinatorial library design and screening strategy, produced Cas9 variants with multiple top immunogenic epitopes simultaneously mutated while still retaining significant genome targeting functionality. Spurred by this, delivery of SpCas9WT and SpCas9v4 and CRISPRoff versions of the same as icRNAs were assessed. CRISPRoff represents one of the newest additions to the CRISPR toolbox with the exciting capability to permanently silence gene expression upon transient expression (85). It was conjectured that wtCas9 and CRISPRoff would represent exciting applications of icRNAs for hit-and-run genome and epigenome targeting, as the prolonged persistence could potentially boost targeting, while the use of partially deimmunized Cas9 proteins would enable greater safety in therapeutic contexts. Specifically, icRNA for WT SpCas9 or SpCas9v4, along with sgRNA targeting the AAVS1 locus, or icRNA for CRISPRoff versions along with sgRNA targeting the B2M gene were transfected into HEK293T (86). Excitingly, both robust genome and epigenome targeting via the icRNA delivery format was observed (FIGS. 11E-11F). Lastly, to assess the specificity of SpCas9v4 targeting, RNA sequencing on WT and SpCas9v4 CRISPRoff samples was performed with and without the B2M guide. As expected, B2M was significantly down-regulated in SpCas9WT and SpCas9v4 samples containing the sgRNA compared to samples with no guide (FIG. 16D, red dot—large dot). Importantly, all differentially expressed genes (DEGs) for V4 were also DEGs for WT, suggesting that SpCas9v4 and SpCas9WT are comparably specific in this assay (FIG. 29D, purple dots—small dots).

### REFERENCES

1. K. Karikó, H. Muramatsu, J. Ludwig, D. Weissman, Generating the optimal mRNA for therapy: HPLC purification eliminates immune activation and improves translation of nucleoside-modified, protein-encoding mRNA. Nucleic Acids Res. 39, e142 (2011).
2. V. Presnyak, N. Alhusaini, Y.-H. Chen, S. Martin, N. Morris, N. Kline, S. Olson, D. Weinberg, K. E. Baker, B. R. Graveley, J. Coller, Codon optimality is a major determinant of mRNA stability. Cell. 160, 1111-1124 (2015).
3. A. N. Kuhn, M. Diken, S. Kreiter, A. Selmi, J. Kowalska, J. Jemielity, E. Darzynkiewicz, C. Huber, O. Türeci, U. Sahin, Phosphorothioate cap analogs increase stability and translational efficiency of RNA vaccines in immature dendritic cells and induce superior immune responses in vivo. Gene Ther. 17, 961-971 (2010).
4. S. Holtkamp, S. Kreiter, A. Selmi, P. Simon, M. Koslowski, C. Huber, Ö. Türeci, U. Sahin, Modification of antigen-encoding RNA increases stability, translational efficacy, and T-cell stimulatory capacity of dendritic cells. Blood. 108 (2006), pp. 4009-4017.
5. A. G. Orlandini von Niessen, M. A. Poleganov, C. Rechner, A. Plaschke, L. M. Kranz, S. Fesser, M. Diken, M. Lower, B. Vallazza, T. Beissert, V. Bukur, A. N. Kuhn, Ö. Türeci, U. Sahin, Improving mRNA-Based Therapeutic Gene Delivery by Expression-Augmenting 3′ UTRs Identified by Cellular Library Screening. Mol. Ther. 27, 824-836 (2019).
6. R. A. Wesselhoeft, P. S. Kowalski, D. G. Anderson, Engineering circular RNA for potent and stable translation in eukaryotic cells. Nat. Commun. 9, 2629 (2018).
7. S. Petkovic, S. Müller, RNA circularization strategies in vivo and in vitro. Nucleic Acids Res. 43, 2454-2465 (2015).
8. S. Müller, B. Appel, In vitro circularization of RNA. RNA Biol. 14, 1018-1027 (2017).
9. R. A. Wesselhoeft, P. S. Kowalski, F. C. Parker-Hale, Y. Huang, N. Bisaria, D. G. Anderson, RNA Circularization Diminishes Immunogenicity and Can Extend Translation Duration In vivo. Mol. Cell. 74, 508-520.e4 (2019).
10. N. Abe, K. Matsumoto, M. Nishihara, Y. Nakano, A. Shibata, H. Maruyama, S. Shuto, A. Matsuda, M. Yoshida, Y. Ito, H. Abe, Rolling Circle Translation of Circular RNA in Living Human Cells. Sci. Rep. 5, 16435 (2015).
11. X. Fan, Y. Yang, C. Chen, Z. Wang, Pervasive translation of circular RNAs driven by short IRES-like elements. bioRxiv (2020), p. 473207.
12. T. B. Hansen, T. I. Jensen, B. H. Clausen, J. B. Bramsen, B. Finsen, C. K. Damgaard, J. Kjems, Natural RNA circles function as efficient microRNA sponges. Nature. 495, 384-388 (2013).
13. W. R. Jeck, N. E. Sharpless, Detecting and characterizing circular RNAs. Nat. Biotechnol. 32, 453-461 (2014).
14. S. Kameda, H. Ohno, H. Saito, Synthetic circular RNA switches and circuits that control protein expression in mammalian cells. Nucleic Acids Res. (2023), doi:10.1093/nar/gkac1252.
15. R. Chen, S. K. Wang, J. A. Belk, L. Amaya, Z. Li, A. Cardenas, B. T. Abe, C.-K. Chen, P. A. Wender, H. Y. Chang, Engineering circular RNA for enhanced protein production. Nat. Biotechnol. (2022), doi:10.1038/s41587-022-01393-0.
16. J. L. Litke, S. R. Jaffrey, Highly efficient expression of circular RNA aptamers in cells using autocatalytic transcripts. Nat. Biotechnol. 37, 667-675 (2019).
17. D. Katrekar, J. Yen, Y. Xiang, A. Saha, D. Meluzzi, Y. Savva, P. Mali, Efficient in vitro and in vivo RNA editing via recruitment of endogenous ADARs using circular guide RNAs. Nature Biotechnology (2022), doi:10.1038/s41587-021-01171-4.
18. A. Li, M. R. Tanner, C. M. Lee, A. E. Hurley, M. De Giorgi, K. E. Jarrett, T. H. Davis, A. M. Doerfler, G. Bao, C. Beeton, W. R. Lagor, AAV-CRISPR Gene Editing Is Negated by Pre-existing Immunity to Cas9. Mol. Iher. 28, 1432-1441 (2020).
19. C. T. Charlesworth, P. S. Deshpande, D. P. Dever, J. Camarena, V. T. Lemgart, M. K. Cromer, C. A. Vakulskas, M. A. Collingwood, L. Zhang, N. M. Bode, M. A. Behlke, B. Dejene, B. Cieniewicz, R. Romano, B. J. Lesch, N. Gomez-Ospina, S. Mantri, M. Pavel-Dinu, K. I. Weinberg, M. H. Porteus, Identification of preexisting adaptive immunity to Cas9 proteins in humans. Nat. Med. 25, 249-254 (2019).
20. N. Chaudhary, D. Weissman, K. A. Whitehead, mRNA vaccines for infectious diseases: principles, delivery and clinical translation. Nat. Rev. Drug Discov. 20, 817-838 (2021).
21. K. S. Corbett, D. K. Edwards, S. R. Leist, O. M. Abiona, S. Boyoglu-Barnum, R. A. Gillespie, S. Himansu, A. Schtafer, C. T. Ziwawo, A. T. DiPiazza, K. H. Dinnon, S. M. Elbashir, C. A. Shaw, A. Woods, E. J. Fritch, D. R. Martinez, K. W. Bock, M. Minai, B. M. Nagata, G. B. Hutchinson, K. Wu, C. Henry, K. Bahl, D. Garcia-Dominguez, L. Ma, I. Renzi, W.-P. Kong, S. D. Schmidt, L. Wang, Y. Zhang, E. Phung, L. A. Chang, R. J. Loomis, N. E. Altaras, E. Narayanan, M. Metkar, V. Presnyak, C. Liu, M. K. Louder, W. Shi, K. Leung, E. S. Yang, A. West, K. L. Gully, L. J. Stevens, N. Wang, D. Wrapp, N. A. Doria-Rose, G. Stewart-Jones, H. Bennett, G. S. Alvarado, M. C. Nason, T. J. Ruckwardt, J. S. McLellan, M. R. Denison, J. D. Chappell, I. N. Moore, K. M. Morabito, J. R. Mascola, R. S. Baric, A. Carfi, B. S. Graham, SARS-CoV-2 mRNA vaccine design enabled by prototype pathogen preparedness. Nature. 586, 567-571 (2020).
22. K. O. Saunders, E. Lee, R. Parks, D. R. Martinez, D. Li, H. Chen, R. J. Edwards, S. Gobeil, M. Barr, K. Mansouri, S. M. Alam, L. L. Sutherland, F. Cai, A. M. Sanzone, M. Berry, K. Manne, K. W. Bock, M. Minai, B. M. Nagata, A. B. Kapingidza, M. Azoitei, L. V. Tse, T. D. Scobey, R. L. Spreng, R. W. Rountree, C. T. DeMarco, T. N. Denny, C. W. Woods, E. W. Petzold, J. Tang, T. H. Oguin 3rd, G. D. Sempowski, M. Gagne, D. C. Douek, M. A. Tomai, C. B. Fox, R. Seder, K. Wiehe, D. Weissman, N. Pardi, H. Golding, S. Khurana, P. Acharya, H. Andersen, M. G. Lewis, I. N. Moore, D. C. Montefiori, R. S. Baric, B. F. Haynes, Neutralizing antibody vaccine for pandemic and pre-emergent coronaviruses. Nature. 594, 553-559 (2021).
23. S. J. Thomas, J. L. Perez, S. P. Lockhart, S. Hariharan, N. Kitchin, R. Bailey, K. Liau, E. Lagkadinou, Ö. Türeci, U. Sahin, X. Xu, K. Koury, S. S. Dychter, C. Lu, T. C. Gentile, W. C. Gruber, Efficacy and safety of the BNT162b2 mRNA COVID-19 vaccine in participants with a history of cancer: subgroup analysis of a global phase 3 randomized clinical trial. Vaccine (2021), doi:10.1016/j.vaccine.2021.12.046.
24. L. V. Zinsli, N. Stierlin, M. J. Loessner, M. Schmelcher, Deimmunization of protein therapeutics—Recent advances in experimental and computational epitope prediction and deletion. Computational and Structural Biotechnology Journal. 19 (2021), pp. 315-329.
25. C. Y. Chen, P. Samow, Initiation of protein synthesis by the eukaryotic translational apparatus on circular RNAs. Science. 268, 415-417 (1995).
26. S. K. Jang, H. G. Krausslich, M. J. Nicklin, G. M. Duke, A. C. Palmenberg, E. Wimmer, A segment of the 5′ nontranslated region of encephalomyocarditis virus RNA directs internal entry of ribosomes during in vitro translation. Journal of Virology. 62 (1988), pp. 2636-2643.
27. C. E. Aitken, J. R. Lorsch, A mechanistic overview of translation initiation in eukaryotes. Nat. Struct. Mol. Biol. 19, 568-576 (2012).
28. G. Alkemar, O. Nygird, Secondary structure of two regions in expansion segments ES3 and ES6 with the potential of forming a tertiary interaction in eukaryotic 40S ribosomal subunits. RNA. 10, 403-411 (2004).
29. P. Bhat, S. Shwetha, D. K. Sharma, A. P. Joseph, N. Srinivasan, S. Das, The beta hairpin structure within ribosomal protein S5 mediates interplay between domains II and IV and regulates HCV IRES function. Nucleic Acids Res. 43, 2888-2901 (2015).
30. J. Chen, A.-D. Brunner, J. Z. Cogan, J. K. Nuflez, A. P. Fields, B. Adamson, D. N. Itzhak, J. Y. Li, M. Mann, M. D. Leonetti, J. S. Weissman, Pervasive functional translation of noncanonical human open reading frames. Science. 367, 1140-1146 (2020).
31. J. W. B. Hershey, N. Sonenberg, M. B. Mathews, Principles of translational control: an overview. Cold Spring Harb. Perspect. Biol. 4 (2012), doi:10.1101/cshperspect.a011528.
32. Y. G. Chen, R. Chen, S. Ahmad, R. Verma, S. P. Kasturi, L. Amaya, J. P. Broughton, J. Kim, C. Cadena, B. Pulendran, S. Hur, H. Y. Chang, N6-Methyladenosine Modification Controls Circular RNA Immunity. Mol. Cell. 76, 96-109.e9 (2019).
33. H. Steinle, A. Behring, C. Schlensak, H. P. Wendel, M. Avci-Adali, Concise Review: Application of In vitro Transcribed Messenger RNA for Cellular Engineering and Reprogramming: Progress and Challenges. Stem Cells. 35, 68-79 (2017).
34. L. Warren, P. D. Manos, T. Ahfeldt, Y.-H. Loh, H. Li, F. Lau, W. Ebina, P. K. Mandal, Z. D. Smith, A. Meissner, G. Q. Daley, A. S. Brack, J. J. Collins, C. Cowan, T. M. Schlaeger, D. J. Rossi, Highly efficient reprogramming to pluripotency and directed differentiation of human cells with synthetic modified mRNA. Cell Stem Cell. 7, 618-630 (2010).
35. J. Yang, S. S. Rajan, M. J. Friedrich, G. Lan, X. Zou, H. Ponstingl, D. A. Garyfallos, P. Liu, A. Bradley, E. Metzakopian, Genome-Scale CRISPRa Screen Identifies Novel Factors for Cellular Reprogramming. Stem Cell Reports. 12, 757-771 (2019).
36. M. Jayaraman, S. M. Ansell, B. L. Mui, Y. K. Tam, J. Chen, X. Du, D. Butler, L. Eltepu, S. Matsuda, J. K. Narayanannair, K. G. Rajeev, I. M. Hafez, A. Akinc, M. A. Maier, M. A. Tracy, P. R. Cullis, T. D. Madden, M. Manoharan, M. J. Hope, Maximizing the potency of siRNA lipid nanoparticles for hepatic gene silencing in vivo. Angew. Chem. Int. Ed Engl. 51, 8529-8533 (2012).
37. S. Sabnis, E. S. Kumarasinghe, T. Salerno, C. Mihai, T. Ketova, J. J. Senn, A. Lynn, A. Bulychev, I. McFadyen, J. Chan, Ö. Almarsson, M. G. Stanton, K. E. Benenato, A Novel Amino Lipid Series for mRNA Delivery: Improved Endosomal Escape and Sustained Pharmacology and Safety in Non-human Primates. Mol. Ther. 26, 1509-1519 (2018).
38. A. Lombardo, P. Genovese, C. M. Beausejour, S. Colleoni, Y.-L. Lee, K. A. Kim, D. Ando, F. D. Umov, C. Galli, P. D. Gregory, M. C. Holmes, L. Naldini, Gene editing in human stem cells using zinc finger nucleases and integrase-defective lentiviral vector delivery. Nat. Biotechnol. 25, 1298-1306 (2007).
39. J. Zou, M. L. Maeder, P. Mali, S. M. Pruett-Miller, S. Thibodeau-Beganny, B.-K. Chou, G. Chen, Z. Ye, I.-H. Park, G. Q. Daley, M. H. Porteus, J. K. Joung, L. Cheng, Gene targeting of a disease-related gene in human induced pluripotent stem and embryonic stem cells. Cell Stem Cell. 5, 97-110 (2009).
40. M. Abifadel, M. Varret, J.-P. Rab s, D. Allard, K. Ouguerram, M. Devillers, C. Cruaud, S. Benjannet, L. Wickham, D. Erlich, A. Derrd, L. Villdger, M. Famier, I. Beucler, E. Bruckert, J. Chambaz, B. Chanu, J.-M. Lecerf, G. Luc, P. Moulin, J. Weissenbach, A. Prat, M. Krempf, C. Junien, N. G. Seidah, C. Boileau, Mutations in PCSK9 cause autosomal dominant hypercholesterolemia. Nat. Genet. 34, 154-156 (2003).
41. K. N. Maxwell, J. L. Breslow, Adenoviral-mediated expression of Pcsk9 in mice results in a low-density lipoprotein receptor knockout phenotype. Proc. Natl. Acad. Sci. U.S.A 101, 7100-7105 (2004).
42. J. C. Cohen, E. Boerwinkle, T. H. Mosley Jr, H. H. Hobbs, Sequence variations in PCSK9, low LDL, and protection against coronary heart disease. N. Engl. J. Med. 354, 1264-1272 (2006).
43. P. I. Thakore, J. B. Kwon, C. E. Nelson, D. C. Rouse, M. P. Gemberling, M. L. Oliver, C. A. Gersbach, RNA-guided transcriptional silencing in vivo with S. aureus CRISPR-Cas9 repressors. Nat. Commun. 9, 1674 (2018).
44. F. A. Ran, L. Cong, W. X. Yan, D. A. Scott, J. S. Gootenberg, A. J. Kriz, B. Zetsche, O. Shalem, X. Wu, K. S. Makarova, E. V. Koonin, P. A. Sharp, F. Zhang, In vivo genome editing using Staphylococcus aureus Cas9. Nature. 520, 186-191 (2015).
45. N.-Y. He, Q. Li, C.-Y. Wu, Z. Ren, Y. Gao, L.-H. Pan, M.-M. Wang, H.-Y. Wen, Z.-S. Jiang, Z.-H. Tang, L.-S. Liu, Lowering serum lipids via PCSK9-targeting drugs: current advances and future perspectives. Acta Pharmacol. Sin. 38, 301-311 (2017).
46. P. M. Ridker, J. Revkin, P. Amarenco, R. Brunell, M. Curto, F. Civeira, M. Flather, R. J. Glynn, J. Gregoire, J. W. Jukema, Y. Karpov, J. J. P. Kastelein, W. Koenig, A. Lorenzatti, P. Manga, U. Masiukiewicz, M. Miller, A. Mosterd, J. Murin, J. C. Nicolau, S. Nissen, P. Ponikowski, R. D. Santos, P. F. Schwartz, H. Soran, H. White, R. S. Wright, M. Vrablik, C. Yunis, C. L. Shear, J.-C. Tardif, SPIRE Cardiovascular Outcome Investigators, Cardiovascular Efficacy and Safety of Bococizumab in High-Risk Patients. N. Engl. J. Med. 376, 1527-1539 (2017).
47. M. S. Sabatine, R. P. Giugliano, A. C. Keech, N. Honarpour, S. D. Wiviott, S. A. Murphy, J. F. Kuder, H. Wang, T. Liu, S. M. Wasserman, P. S. Sever, T. R. Pedersen, FOURIER Steering Committee and Investigators, Evolocumab and Clinical Outcomes in Patients with Cardiovascular Disease. N. Engl. J. Med. 376, 1713-1722 (2017).
48. K. Fitzgerald, S. White, A. Borodovsky, B. R. Bettencourt, A. Strahs, V. Clausen, P. Wijngaard, J. D. Horton, J. Taubel, A. Brooks, C. Fernando, R. S. Kauffman, D. Kallend, A. Vaishnaw, A. Simon, A Highly Durable RNAi Therapeutic Inhibitor of PCSK9. N. Engl. J. Med. 376, 41-51 (2017).
49. Q. Ding, A. Strong, K. M. Patel, S.-L. Ng, B. S. Gosis, S. N. Regan, C. A. Cowan, D. J. Rader, K. Musunuru, Permanent alteration of PCSK9 with in vivo CRISPR-Cas9 genome editing. Circ. Res. 115, 488-492 (2014).
50. A. M. Moreno, N. Palmer, F. Alemkn, G. Chen, A. Pla, N. Jiang, W. L. Chew, M. Law, P. Mali, Author Correction: Immune-orthogonal orthologues of AAV capsids and of Cas9 circumvent the immune response to the administration of gene therapy. Nat Biomed Eng. 3, 842 (2019).
51. W. L. Chew, M. Tabebordbar, J. K. W. Cheng, P. Mali, E. Y. Wu, A. H. M. Ng, K. Zhu, A. J. Wagers, G. M. Church, A multifunctional AAV-CRISPR-Cas9 and its host response. Nat. Methods. 13, 868-874 (2016).
52. V. Jawa, F. Terry, J. Gokemeijer, S. Mitra-Kaushik, B. J. Roberts, S. Tourdot, A. S. De Groot, T-Cell Dependent Immunogenicity of Protein Therapeutics Pre-clinical Assessment and Mitigation-Updated Consensus and Review 2020. Frontiers in Immunology. 11 (2020), doi:10.3389/fimmu.2020.01301.
53. F. Moghadam, R LeGraw, J. J. Velazquez, N. C. Yeo, C. Xu, J. Park, A. Chavez, M. R. Ebrahimkhani, S. Kiani, Synthetic immunomodulation with a CRISPR super-repressor in vivo. Nat. Cell Biol. 22, 1143-1154 (2020).
54. S. R Ferdosi, R Ewaisha, F. Moghadam, S. Krishna, J. G. Park, M. R Ebrahimkhani, S. Kiani, K. S. Anderson, Multifunctional CRISPR-Cas9 with engineered immunosilenced human T cell epitopes. Nat. Commun. 10, 1842 (2019).
55. B. D. Allen, A. Nisthal, S. L. Mayo, Experimental library screening demonstrates the successful application of computational protein design to large structural ensembles. Proc. Natl. Acad. Sci. U.S.A 107, 19838-19843 (2010).
56. M. G. F. Sun, M.-H. Seo, S. Nim, C. Corbi-Verge, P. M. Kim, Protein engineering by highly parallel screening of computationally designed variants. Sci Adv. 2, e1600692 (2016).
57. J. Cao, E. M. Novoa, Z. Zhang, W. C. W. Chen, D. Liu, G. C. G. Choi, A. S. L. Wong, C. Wehrspaun, M. Kellis, T. K. Lu, High-throughput 5′ UTR engineering for enhanced protein production in non-viral gene therapies. Nat. Commun. 12, 1-10 (2021).
58. J. H. Hu, S. M. Miller, M. H. Geurts, W. Tang, L. Chen, N. Sun, C. M. Zeina, X. Gao, H. A. Rees, Z. Lin, D. R Liu, Evolved Cas9 variants with broad PAM compatibility and high DNA specificity. Nature. 556, 57-63 (2018).
59. R T. Walton, K. A. Christie, M. N. Whittaker, B. P. Kleinstiver, Unconstrained genome targeting with near-PAMless engineered CRISPR-Cas9 variants. Science. 368, 290-296 (2020).
60. B. P. Kleinstiver, M. S. Prew, S. Q. Tsai, V. V. Topkar, N. T. Nguyen, Z. Zheng, A. P. W. Gonzales, Z. Li, R. T. Peterson, J.-R J. Yeh, M. J. Aryee, J. K. Joung, Engineered CRISPR-Cas9 nucleases with altered PAM specificities. Nature. 523, 481-485 (2015).
61. B. P. Kleinstiver, V. Pattanayak, M. S. Prew, S. Q. Tsai, N. T. Nguyen, Z. Zheng, J. K. Joung, High-fidelity CRISPR-Cas9 nucleases with no detectable genome-wide off-target effects. Nature. 529, 490-495 (2016).
62. E. J. Charles, S. E. Kim, G. J. Knott, D. Smock, J. Doudna, D. F. Savage, Engineering improved Cas13 effectors for targeted post-transcriptional regulation of gene expression. bioRxiv (2021), p. 2021.05.26.445687.
63. K. E. Griswold, C. Bailey-Kellogg, Design and engineering of deimmunized biotherapeutics. Curr. Opin. Struct. Biol. 39, 79-88 (2016).
64. M. B. Doud, J. M. Lee, J. D. Bloom, How single mutations affect viral escape from broad and narrow antibodies to H1 influenza hemagglutinin. Nat. Commun. 9, 1386 (2018).
65. G. Gasiunas, J. K. Young, T. Karvelis, D. Kazlauskas, T. Urbaitis, M. Jasnauskaite, M. M. Grusyte, S. Paulraj, P.-H. Wang, Z. Hou, S. K. Dooley, M. Cigan, C. Alarcon, N. D. Chilcoat, G. Bigelyte, J. L. Curcuru, M. Mabuchi, Z. Sun, R. T. Fuchs, E. Schildkraut, P. R. Weigele, W. E. Jack, G. B. Robb, C. Venclovas, V. Siksnys, A catalogue of biochemically diverse CRISPR-Cas9 orthologs. Nat. Commun. 11, 5512 (2020).
66. N. Takeuchi, Y. I. Wolf, K. S. Makarova, E. V. Koonin, Nature and intensity of selection pressure on CRISPR-associated genes. J. Bacteriol. 194, 1216-1225 (2012).
67. M. Andreatta, M. Nielsen, Gapped sequence alignment using artificial neural networks: application to the MHC class I system. Bioinformatics. 32, 511-517 (2016).
68. M. Nielsen, C. Lundegaard, P. Woming, S. L. Lauemoller, K. Lamberth, S. Buus, S. Brunak, O. Lund, Reliable prediction of T-cell epitopes using neural networks with novel sequence representations. Protein Sci. 12, 1007-1017 (2003).
69. D. C. Osipovitch, A. S. Parker, C. D. Makokha, J. Desrosiers, W. C. Kett, L. Moise, C. Bailey-Kellogg, K. E. Griswold, Design and analysis of immune-evading enzymes for ADEPT therapy. Protein Eng. Des. Sel. 25, 613-623 (2012).
70. Y. Choi, D. Verma, K. E. Griswold, C. Bailey-Kellogg, “EpiSweep: Computationally Driven Reengineering of Therapeutic Proteins to Reduce Immunogenicity While Maintaining Function” in Computational Protein Design, I. Samish, Ed. (Springer New York, New York, NY, 2017), pp. 375-398.
71. C. King, E. N. Garza, R. Mazor, J. L. Linehan, I. Pastan, M. Pepper, D. Baker, Removing T-cell epitopes with computational protein design. Proc. Natl. Acad. Sci. U.S.A 111, 8577-8582 (2014).
72. R. Mazor, D. Crown, S. Addissie, Y. Jang, G. Kaplan, I. Pastan, Elimination of murine and human T-cell epitopes in recombinant immunotoxin eliminates neutralizing and anti-drug antibodies in vivo. Cell. Mol. Immunol. 14, 432-442 (2017).
73. Y. Wang, Y. Zhao, A. Bollas, Y. Wang, K. F. Au, Nanopore sequencing technology, bioinformatics and applications. Nat. Biotechnol. 39, 1348-1365 (2021).
74. F. J. Rang, W. P. Kloosterman, J. de Ridder, From squiggle to basepair: computational approaches for improving nanopore sequencing read accuracy. Genome Biol. 19, 90 (2018).
75. B. Schubert, C. Scharfe, P. Dönnes, T. Hopf, D. Marks, O. Kohlbacher, Population-specific design of de-immunized protein biotherapeutics. PLoS Comput. Biol. 14, e1005983 (2018).
76. S. Liao, M. Tammaro, H. Yan, Enriching CRISPR-Cas9 targeted cells by co-targeting the HPRT gene. Nucleic Acids Res. 43, e134 (2015).
77. F. Yang, H. Fang, D. Wang, Y. Chen, Y. Zhai, B.-B. S. Zhou, H. Li, HPRT1 activity loss is associated with resistance to thiopurine in ALL. Oncotarget. 9, 2268-2278 (2018).
78. M.-R. Meini, P. E. Tomatis, D. M. Weinreich, A. J. Vila, Quantitative Description of a Protein Fitness Landscape Based on Molecular Features. Mol. Biol. Evol. 32, 1774-1787 (2015).
79. P. Mali, L. Yang, K. M. Esvelt, J. Aach, M. Guell, J. E. DiCarlo, J. E. Norville, G. M. Church, RNA-guided human genome engineering via Cas9. Science. 339, 823-826 (2013).
80. K. Clement, H. Rees, M. C. Canver, J. M. Gehrke, R. Farouni, J. Y. Hsu, M. A. Cole, D. R. Liu, J. K. Joung, D. E. Bauer, L. Pinello, CRISPResso2 provides accurate and rapid genome editing sequence analysis. Nat. Biotechnol. 37, 224-226 (2019).
81. T. Ninkovic, L. Kinarsky, K. Engelmann, V. Pisarev, S. Sherman, O. J. Finn, F.-G. Hanisch, Identification of O-glycosylated decapeptides within the MUC1 repeat domain as potential MHC class I (A2) binding epitopes. Mol. Immunol. 47, 131-140 (2009).
82. J. K. Etschel, A. G. Hilckelhoven, C. Hofmann, K. Zitzelsberger, K. Maurer, S. Bergmann, S. M. Mueller-Schmucker, J. Wittmann, B. M. Spriewald, J. Dörrie, N. Schaft, T. Harrer, HIV-1 mRNA electroporation of PBMC: a simple and efficient method to monitor T-cell responses against autologous HIV-1 in HIV-1-infected patients. J. Immunol. Methods. 380, 40-55 (2012).
83. K. Van Camp, N. Cools, B. Stein, A. Van de Velde, H. Goossens, Z. N. Bememan, V. Van Tendeloo, Efficient mRNA electroporation of peripheral blood mononuclear cells to detect memory T cell responses for immunomonitoring purposes. J. Immunol. Methods. 354, 1-10 (2010).
84. A. M. Moreno, X. Fu, J. Zhu, D. Katrekar, Y.-R. V. Shih, J. Marlett, J. Cabotaje, J. Tat, J. Naughton, L. Lisowski, S. Varghese, K. Zhang, P. Mali, In situ Gene Therapy via AAV-CRISPR-Cas9-Mediated Targeted Gene Regulation. Mol. Ther. 28, 1931 (2020).
85. J. K. Nufnez, J. Chen, G. C. Pommier, J. Z. Cogan, J. M. Replogle, C. Adriaens, G. N. Ramadoss, Q. Shi, K. L. Hung, A. J. Samelson, A. N. Pogson, J. Y. S. Kim, A. Chung, M. D. Leonetti, H. Y. Chang, M. Kampmann, B. E. Bernstein, V. Hovestadt, L. A. Gilbert, J. S. Weissman, Genome-wide programmable transcriptional memory by CRISPR-based epigenome editing. Cell. 184, 2503-2519.e17 (2021).
86. A. Amabile, A. Migliara, P. Capasso, M. Biffi, D. Cittaro, L. Naldini, A. Lombardo, Inheritable Silencing of Endogenous Genes by Hit-and-Run Targeted Epigenetic Editing. Cell. 167, 219-232.e14 (2016).
87. C.-K. Chen, R. Cheng, J. Demeter, J. Chen, S. Weingarten-Gabbay, L. Jiang, M. P. Snyder, J. S. Weissman, E. Segal, P. K. Jackson, H. Y. Chang, Structured elements drive extensive circular RNA translation. Mol. Cell. 81, 4300-4318.e13 (2021).
88. Y. Yang, X. Fan, M. Mao, X. Song, P. Wu, Y. Zhang, Y. Jin, Y. Yang, L.-L. Chen, Y. Wang, C. C. Wong, X. Xiao, Z. Wang, Extensive translation of circular RNAs driven by N6-methyladenosine. Cell Res. 27, 626-641 (2017).
89. K. D. Meyer, D. P. Patil, J. Zhou, A. Zinoviev, M. A. Skabkin, O. Elemento, T. V. Pestova, S.-B. Qian, S. R. Jaffrey, 5′ UTR m6A Promotes Cap-Independent Translation. Cell. 163, 999-1010 (2015).
90. S. Weingarten-Gabbay, S. Elias-Kirma, R. Nir, A. A. Gritsenko, N. Stem-Ginossar, Z. Yakhini, A. Weinberger, E. Segal, Comparative genetics. Systematic discovery of cap-independent translation sequences in human and viral genomes. Science. 351 (2016), doi:10.1126/science.aad4939.
91. P. J. Sample, B. Wang, D. W. Reid, V. Presnyak, I. J. McFadyen, D. R. Morris, G. Seelig, Human 5′ UTR design and variant effect prediction from a massively parallel translation assay. Nat. Biotechnol. 37, 803-809 (2019).
92. Y. G. Chen, M. V. Kim, X. Chen, P. J. Batista, S. Aoyama, J. E. Wilusz, A. Iwasaki, H. Y. Chang, Sensing Self and Foreign Circular RNAs by Intron Identity. Mol. Cell. 67, 228-238.e5 (2017).
93. B. T. Abe, R. Alexander Wesselhoeft, R. Chen, D. G. Anderson, H. Y. Chang, Circular RNA migration in agarose gel electrophoresis. Molecular Cell (2022), doi:10.1016/j.molcel.2022.03.008.
94. M. A. Stiffler, F. J. Poelwijk, K. P. Brock, R. R. Stein, A. Riesselman, J. Teyra, S. S. Sidhu, D. S. Marks, N. P. Gauthier, C. Sander, Protein Structure from Experimental Evolution. Cell Syst. 10, 15-24.e5 (2020).
95. A. G. Green, H. Elhabashy, K. P. Brock, R. Maddamsetti, O. Kohlbacher, D. S. Marks, Large-scale discovery of protein interactions at residue resolution using co-evolution calculated from genomic sequences. Nature Communications. 12 (2021), doi:10.1038/s41467-021-21636-z.
96. K. Saylor, F. Gillam, T. Lohneis, C. Zhang, Designs of Antigen Structure and Composition for Improved Protein-Based Vaccine Efficacy. Front. Immunol. 11, 283 (2020).
97. A. V. Joglekar, M. T. Leonard, J. D. Jeppson, M. Swift, G. Li, S. Wong, S. Peng, J. M. Zaretsky, J. R. Heath, A. Ribas, M. T. Bethune, D. Baltimore, T cell antigen discovery via signaling and antigen-presenting bifunctional receptors. Nat. Methods. 16, 191-198 (2019).
98. X. Lian, J. Zhang, S. M. Azarin, K. Zhu, L. B. Hazeltine, X. Bao, C. Hsiao, T. J. Kamp, S. P. Palecek, Directed cardiomyocyte differentiation from human pluripotent stem cells by modulating Wnt/β-catenin signaling under fully defined conditions. Nat. Protoc. 8, 162-175 (2013).
99. A. Kumar, S. K. Thomas, K. C. Wong, V. Lo Sardo, D. S. Cheah, Y.-H. Hou, J. K. Placone, K. P. Tenerelli, W. C. Ferguson, A. Torkamani, E. J. Topol, K. K. Baldwin, A. J. Engler, Mechanical activation of noncoding-RNA-mediated regulation of disease-associated phenotypes in human cardiomyocytes. Nat Biomed Eng. 3, 137-146 (2019).

100. D. Chen, K. T. Love, Y. Chen, A. A. Eltoukhy, C. Kastrup, G. Sahay, A. Jeon, Y. Dong, K. A. Whitehead, D. G. Anderson, Rapid discovery of potent siRNA-containing lipid nanoparticles enabled by controlled microfluidic formulation. J. Am. Chem. Soc. 134, 6948-6951 (2012).

101. N. M. Belliveau, J. Huft, P. J. Lin, S. Chen, A. K. Leung, T. J. Leaver, A. W. Wild, J. B. Lee, R. J. Taylor, Y. K. Tam, C. L. Hansen, P. R. Cullis, Microfluidic Synthesis of Highly Potent Limit-size Lipid Nanoparticles for In vivo Delivery of siRNA. Mol. Ther. Nucleic Acids. 1, e37 (2012).

It will be understood that various modifications may be made without departing from the spirit and scope of this disclosure. Accordingly, other embodiments are within the scope of the following claims.

What is claimed is:

1. A linearized ribozyme activated RNA construct comprising from 5′ to 3′ end:
(a) a first ligation sequence;
(b) an IRES sequence;
(c) a polynucleotide sequence of interest encoding a recombinant polypeptide;
(d) a 3′ UTR sequence;
(e) a poly(A) sequence; and
(f) a second ligation sequence,
wherein the first ligation sequence comprises a 5′-OH end, the second ligation sequence comprises a 2′, 3′-cyclic phosphate end,
wherein the first and second ligation sequences form a stem substrate for an RNA ligase.

2. The linearized ribozyme activated RNA construct of claim 1, wherein the IRES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348), wherein the T nucleotides are U nucleotides in the RNA construct.

3. The linearized ribozyme activated RNA construct of claim 1 or 2, wherein the 3′ UTR sequence is selected from the group consisting of an mtRNR1-AES 3′ UTR (SEQ ID NO: 1354), an mtRNR1-LSP1 3′ UTR (SEQ ID NO: 1355), an AES-mtRNR1 3′ UTR (SEQ ID NO: 1356), an AES-hBg 3′ UTR (SEQ ID NO: 1357), an FCGRT-hBg 3′ UTR (SEQ ID NO: 1358), a 2hBg 3′ UTR (SEQ ID NO: 1359), and a HBA1 3′ UTR (SEQ ID NO: 1360), wherein the T nucleotides are U nucleotides in the RNA construct.

4. The linearized ribozyme activated RNA construct of any one of claims 1-3, wherein the 3′ UTR sequence further comprises a WPRE sequence.

5. The linearized ribozyme activated RNA construct of claim 4, wherein the WPRE sequence comprises the nucleic acid sequence of SEQ ID NO: 1353.

6. The linearized ribozyme activated RNA construct of claim 4 or 5, wherein the poly(A) sequence positioned 3′ of the WPRE sequence.

7. The linearized ribozyme activated RNA construct of any one of claims 1-6, wherein the poly(A) sequence has a length ranging from about 5 to about 1000 adenine nucleotides.

8. The linearized ribozyme activated RNA construct of any one of claims 1-7, wherein the poly(A) sequence has a length ranging from about 5 to about 300 adenine nucleotides.

9. The linearized ribozyme activated RNA construct of any one of claims 1-8, wherein a portion of the first ligation sequence is complementary to a portion of the second ligation sequence.

10. The linearized ribozyme activated RNA construct of any one of claims 1-9, wherein the first and/or the second ligation sequence comprises a nucleic acid sequence having at least 90% sequence identity of 5′-AACCAUGCCGACUGAUGGCAG-3′ (SEQ ID NO: 1413).

11. The linearized ribozyme activated RNA construct of any one of claims 1-10, wherein the first and/or the second ligation sequence comprises a nucleic acid sequence having at least 90% sequence identity of 5′-CUGCCAUCAGUCGGCGUGGACUGUAG-3′ (SEQ ID NO: 1415).

12. The linearized ribozyme activated RNA construct of any one of claims 1-11, wherein the construct lacks a ribozyme.

13. The linearized ribozyme activated RNA construct of any one of claims 1-12, wherein the construct comprises one or more modified nucleic acids.

14. The linearized ribozyme activated RNA construct of any one of claims 1-13, wherein the construct capable of being introduced into a cell.

15. The linearized ribozyme activated RNA construct of any one of claims 1-13, wherein the construct has been introduced into a cell.

16. An engineered cell comprising any one of the linearized ribozyme activated RNA constructs of any one of claims 1-13.

17. The engineered cell of claim 16, further comprising a circular RNA construct formed from the linearized ribozyme activated RNA construct.

18. The engineered cell of claim 16 or 17, wherein the cell lacks a DNA construct encoding the linearized ribozyme activated RNA construct.

19. The engineered cell of any one of claims 16-18, wherein the cell is a eukaryotic cell.

20. The engineered cell of claim 19, wherein the eukaryotic cell is a mammalian cell.

21. The engineered cell of claim 20, wherein the mammalian cell is a human cell.

22. A method for producing an engineered cell comprising a circular RNA comprising: introducing the linearized ribozyme activated RNA construct of any one of claims 1-13 into the cell, wherein an RNA ligase in the cell ligates the first and second ligation sequences, thereby forming the circular RNA construct.

23. The method of claim 22, wherein the RNA ligase is an endogenous RtcB ligase.

24. The method of claim 22 or 23, wherein the cell is a eukaryotic cell.

25. The method of claim 24, wherein the eukaryotic cell is a mammalian cell.

26. The method of claim 25, wherein the mammalian cell is a human cell.

27. A method for producing a circular RNA construct comprising contacting the linearized ribozyme activated RNA construct of any one of claims 1-13 with an RNA ligase.

28. The method of claim 27, wherein the contacting is in vitro.

29. The method of claim 27, wherein the contacting is inside a cell.

30. A composition comprising the linearized ribozyme activated RNA construct of any one of claims 1-13 and a delivery system.

31. The composition of claim 30, wherein the delivery system comprises any one selected from the group consisting of a lipid nanoparticle, a liposome, a charged polymer, an uncharged polymer, a nanoparticle, a surfactant, a penetrating enhancer, a gene transfer agent, a phospholipid, a micelle, a synthetic vector, a macromolecule, a dendrimer, a biopolymer, a viral particle, and any combination thereof.

32. The composition of claim 30 or 31, wherein the composition is administered to a subject.

33. The composition of claim 32, wherein the subject is a human subject.

34. A therapeutic composition comprising the linearized ribozyme activated RNA construct of any one of claims 1-13 and a lipid nanoparticle, wherein the lipid nanoparticle comprises: (6Z,9Z,28Z,31Z)-heptatriaconta-6,9,28,31-tetraen-19-yl-4-(dimethylamino) butanoate (DLin-MC3-DMA); cholesterol; 1,2-distearoyl-sn-glycero-3-phosphocholine (DSPC); and 1,2-dimyristoyl-rac-glycero-3-methoxypolyethylene glycol-2000 (DMG-PEG-2000) at a mole ratio of 50:38.5:10:1.5, respectively, and the lipid nanoparticle has an N/P ratio of 5.4.

35. A linearized ribozyme-RNA construct comprising from 5′ to 3′ end:
(a) a first twister ribozyme;
(b) a first ligation sequence;
(c) an IRES sequence;
(d) a polynucleotide sequence of interest encoding a recombinant polypeptide;
(e) a 3′ UTR sequence;
(f) a poly(A) sequence;
(g) a second ligation sequence; and
(h) a second twister ribozyme.

36. The linearized ribozyme-RNA construct of claim 35, wherein the IRES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348), wherein the T nucleotides are U nucleotides in the linearized ribozyme-RNA construct.

37. The linearized ribozyme-RNA construct of claim 35 or 36, wherein the 3′ UTR sequence is selected from the group consisting of an mtRNR1-AES 3′ UTR (SEQ ID NO: 1354), an mtRNR1-LSP1 3′ UTR (SEQ ID NO: 1355), an AES-mtRNR1 3′ UTR (SEQ ID NO: 1356), an AES-hBg 3′ UTR (SEQ ID NO: 1357), an FCGRT-hBg 3′ UTR (SEQ ID NO: 1358), a 2hBg 3′ UTR (SEQ ID NO: 1359), and a HBA1 3′ UTR (SEQ ID NO: 1360), wherein the T nucleotides are U nucleotides in the linearized ribozyme-RNA construct.

38. The linearized ribozyme-RNA construct of any one of claims 35-37, wherein the 3′ UTR sequence further comprises a WPRE sequence.

39. The linearized ribozyme-RNA construct of claim 38, wherein the WPRE sequence comprises the nucleic acid sequence of SEQ ID NO: 1353, wherein the T nucleotides are U nucleotides in the linearized ribozyme-RNA construct.

40. The linearized ribozyme-RNA construct of claim 38 or 39, wherein the poly(A) sequence positioned 3′ of the WPRE sequence.

41. The linearized ribozyme-RNA construct of any one of claims 35-40, wherein the poly(A) sequence has a length ranging from about 5 to about 1000 adenine nucleotides.

42. The linearized ribozyme-RNA construct of any one of claims 35-41, wherein the poly(A) sequence has a length ranging from about 5 to about 300 adenine nucleotides.

43. The linearized ribozyme-RNA construct of any one of claims 35-42, wherein the first and/or second ribozyme is selected from the group consisting a twister ribozyme, twister sister (TS) ribozyme, a hammerhead ribozyme, a hairpin ribozyme, a hepatitis delta virus (HDV) ribozyme, a Varkud satellite (VS) ribozyme, a glucosamine-6-phosphate (GlmS) ribozyme, a pistol ribozyme, and a hatchet ribozyme.

44. The linearized ribozyme-RNA construct of any one of claims 35-43, wherein the first ribozyme and the second ribozyme are the same twister ribozyme.

45. The linearized ribozyme-RNA construct of any one of claims 35-44, wherein the first twister ribozyme and/or the second twister ribozyme is a P1 twister ribozyme.

46. The linearized ribozyme-RNA construct of any one of claims 35-45, wherein the first ribozyme and/or the second ribozyme is a P3 twister ribozyme.

47. The linearized ribozyme-RNA construct of any one of claims 35-46, wherein the first twister ribozyme and/or the second twister ribozyme comprises a nucleic acid sequence having at least 90% sequence identity of
(SEQ ID NO: 1412)
5′-GCCAUCAGUCGCCGGUCCCAAGCCCGGAUAAAAUGGGAGGGGGGGGA
AACCGCCU-3′.

48. The linearized ribozyme-RNA construct of any one of claims 35-47, wherein the first twister ribozyme and/or the second twister ribozyme comprises a nucleic acid sequence having at least 90% sequence identity of
(SEQ ID NO: 1414)
5′-AACACUGCCAAUGCCGGUCCCAAGCCCGGAUAAAAGTGGAGGGUACA
GUCCACGC-3′.

49. The linearized ribozyme-RNA construct of any one of claims 35-48, wherein a portion of the first ligation sequence is complementary to a portion of the first twister ribozyme and a portion of the second ligation sequence is complementary to a portion of the second twister ribozyme

50. The linearized ribozyme-RNA construct of claim 49, wherein the portion of the first ligation sequence that is complementary to the portion of the first twister ribozyme is also complementary to the portion of the second ligation sequence that is complementary to the portion of the second twister ribozyme.

51. The linearized ribozyme-RNA construct of any one of claims 35-50, wherein the first and/or the second ligation sequence comprises a nucleic acid sequence having at least 90% sequence identity of 5′-AACCAUGCCGACUGAUGGCAG-3′ (SEQ ID NO: 1413).

52. The linearized ribozyme-RNA construct of any one of claims 35-51, wherein the first and/or the second ligation sequence comprises a nucleic acid sequence having at least 90% sequence identity of 5′-CUGCCAUCAGUCGGCGUGGACUGUAG-3′ (SEQ ID NO: 1415).

53. The linearized ribozyme-RNA construct of any one of claims 35-52, wherein the construct comprises one or more modified nucleic acids.

54. A DNA construct comprising a RNA polymerase II promoter and a nucleic acid sequence encoding a ribozyme-RNA construct, wherein the ribozyme-RNA construct comprises from 5′ to 3′ end:
(a) a first twister ribozyme;
(b) a first ligation sequence;
(c) an IRES sequence;
(d) a polynucleotide sequence of interest encoding a recombinant polypeptide;
(e) a 3′ UTR sequence;
(f) a poly(A) sequence;
(g) a second ligation sequence; and
(h) a second twister ribozyme,
wherein promoter is operably linked to the nucleic acid sequence encoding the ribozyme-RNA construct.

55. The DNA construct of claim 54, wherein the IRES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348).

56. The DNA construct of claim 54 or 55, wherein the 3′ UTR sequence is selected from the group consisting of mtRNR1-AES 3′ UTR (SEQ ID NO: 1354), mtRNR1-LSP1 3′ UTR (SEQ ID NO: 1355), AES-mtRNR1 3′ UTR (SEQ ID NO: 1356), AES-hBg 3′ UTR (SEQ ID NO: 1357), FCGRT-hBg 3′ UTR (SEQ ID NO: 1358), 2hBg 3′ UTR (SEQ ID NO: 1359), and HBA1 3′ UTR (SEQ ID NO: 1360).

57. The DNA construct of any one of claims 54-56, wherein the 3′ UTR sequence further comprises a WPRE sequence.

58. The DNA construct of claim 57, wherein the WPRE sequence comprises the nucleic acid sequence of SEQ ID NO: 1353.

59. The DNA construct of any one of claims 54-56, wherein the poly(A) sequence is positioned 3′ of the WPRE sequence.

60. The DNA construct of any one of claims 54-59, wherein the poly(A) sequence has a length ranging from about 5 to about 1000 adenine nucleotides.

61. The DNA construct of any one of claims 54-60, wherein the poly(A) sequence has a length ranging from about 5 to about 300 adenine nucleotides.

62. The DNA construct of any one of claims 54-61, wherein the first and/or second ribozyme is selected from the group consisting a twister ribozyme, twister sister (TS) ribozyme, a hammerhead ribozyme, a hairpin ribozyme, a hepatitis delta virus (HDV) ribozyme, a Varkud satellite (VS) ribozyme, a glucosamine-6-phosphate (GlmS) ribozyme, a pistol ribozyme, and a hatchet ribozyme.

63. The DNA construct of any one of claims 54-62, wherein the first ribozyme and the second ribozyme are the same twister ribozyme.

64. The DNA construct of any one of claims 54-63, wherein the first Twister ribozyme and/or the second twister ribozyme is a P1 twister ribozyme.

65. The DNA construct of any one of claims 54-64, wherein the first ribozyme and/or the second ribozyme is a P3 twister ribozyme.

66. The DNA construct of any one of claims 54-65, wherein the first twister ribozyme and/or the second twister ribozyme comprises a nucleic acid sequence having at least 90% sequence identity of
(SEQ ID NO: 1349)
5′-GCCATCAGTCGCCGGTCCCAAGCCCGGATAAAATGGGAGGGGGGGGA
AACCGCCT-3′.

67. The DNA construct of any one of claims 54-66, wherein the first twister ribozyme and/or the second twister ribozyme comprises a nucleic acid sequence having at least 90% sequence identity of 5′-AACACTGCCAATGCCGGTCCCAAGCCCGGATAAAAGTGGAGGGTACAGTCCACGC-3′ (SEQ ID NO: 1350).

68. The DNA construct of any one of claims 54-67, wherein a portion of the first ligation sequence is complementary to a portion of the first twister ribozyme and a portion of the second ligation sequence is complementary to a portion of the second twister ribozyme

69. The DNA construct of claim 68, wherein the portion of the first ligation sequence that is complementary to the portion of the first twister ribozyme is complementary to the portion of the second ligation sequence that is complementary to the portion of the second twister ribozyme.

70. The DNA construct of any one of claims 54-69, wherein the first and/or the second ligation sequence comprises a nucleic acid sequence having at least 90% sequence identity of 5′-AACCATGCCGACTGATGGCAG-3′ (SEQ ID NO:1351).

71. The DNA construct of any one of claims 54-70, wherein the first and/or the second ligation sequence comprises a nucleic acid sequence having at least 90% sequence identity of 5′-CTGCCATCAGTCGGCGTGGACTGTAG-3′ (SEQ ID NO: 1352).

72. A cell comprising the DNA construct of any one of claims 54-71.

73. The cell of claim 72, wherein the cell is a eukaryotic cell.

74. The cell of claim 73, wherein the eukaryotic cell is a mammalian cell.

75. The cell of claim 73 or 74, wherein the eukaryotic cell is a human cell.

76. A cell comprising a circular RNA construct, wherein the circular RNA construct comprises:
(a) a first ligation sequence;
(b) an IRES sequence positioned 3′ of the first ligation sequence;
(c) a polynucleotide sequence of interest encoding a recombinant polypeptide and positioned 3′ of the IRES sequence;
(d) a 3′ UTR sequence positioned 3′ of the IRES sequence;
(e) a poly(A) sequence positioned 3′ of the 3′ UTR; and
(f) a second ligation sequence positioned 3′ of the poly (A) sequence,
wherein the first and second ligation sequences are ligated together.

77. The cell of claim 76, wherein the cell is a mammalian cell.

78. The cell of claim 76 or 77, wherein the cell is a human cell.

79. The cell of any one of claims 76-78, wherein the cell produces an elevated level of the recombinant polypeptide as compared to a corresponding wild-type cell.

80. The cell of any one of claims 76-79, wherein the first and second ligation sequences are ligated together in the cell by an endogenous RNA ligase.

81. The cell of any one of claims 76-80, wherein the IRES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348), wherein the T nucleotides are U nucleotides in the RNA construct.

82. The cell of any one of claims 76-81, wherein the 3′ UTR sequence is selected from the group consisting of mtRNR1-AES 3′ UTR (SEQ ID NO: 1354), mtRNR1-LSP1 3′ UTR (SEQ ID NO: 1355), AES-mtRNR1 3′ UTR (SEQ ID NO: 1356), AES-hBg 3′ UTR (SEQ ID NO: 1357), FCGRT-hBg 3′ UTR (SEQ ID NO: 1358), 2hBg 3′ UTR (SEQ ID NO: 1359), and HBA1 3′ UTR (SEQ ID NO: 1360), wherein the T nucleotides are U nucleotides in the RNA construct.

83. The cell of any one of claims 76-82, wherein the 3′ UTR sequence comprises a WPRE sequence.

84. The cell of any claim 83, wherein the WPRE sequence comprises the nucleic acid sequence of SEQ ID NO: 1353, wherein the T nucleotides are U nucleotides in the RNA construct.

85. The cell of claim 83 or 84, wherein the poly(A) sequence is positioned 3′ of the WPRE sequence.

86. The cell of any one of claims 76-85, wherein the poly(A) sequence has a length ranging from about 5 to about 1000 adenine nucleotides.

87. The cell of any one of claims 76-86, wherein the poly(A) sequence has a length ranging from about 5 to about 300 adenine nucleotides.

88. The cell of any one of claims 76-87, wherein a portion of the first ligation sequence is complementary to a portion of the second ligation sequence.

89. The cell of any one of claims 76-88, wherein the first and/or the second ligation sequence comprises a nucleic acid sequence having at least 90% sequence identity of 5′-AACCAUGCCGACUGAUGGCAG-3′ (SEQ ID NO: 1413).

| (SEQ ID NO: 1413) | |
|---|---|
| | 5′-AACCAUGCCGACUGAUGGCAG-3′. |

90. The cell of any one of claims 76-89, wherein the first and/or the second ligation sequence comprises a nucleic acid sequence having at least 90% sequence identity of

| (SEQ ID NO: 1415) | |
|---|---|
| | 5′-CUGCCAUCAGUCGGCGUGGACUGUAG-3′. |

91. The cell of any one of claims 76-90, wherein the circular RNA construct comprises one or more modified nucleic acids.

92. The cell of any one of claims 76-91, wherein the cell is an engineered cell.

93. The cell of any one of claims 76-92, wherein the cell lacks the DNA construct of any one of claims 52-68, lacks the linearized ribozyme-RNA construct of any one of claims 34-51, or lacks the DNA construct of any one of claims 52-68 and the linearized ribozyme-RNA construct.

94. A ribozyme RNA-construct(s) comprising from 5′ to 3′:
an optional primer region,
an optional barcode region,
a first ribozyme domain,
a first ligation stem domain,
a payload domain,
a second ligation stem domain, and
a second ribozyme domain;
wherein the payload domain comprises from 5′ to 3′:
an internal ribosome entry site (IRES) or a P2A peptide coding sequence,
a coding sequence of at least one polypeptide and/or nucleic acid of interest, and
a 3′UTR sequence;
wherein the transcription of the payload domain is activated by or dependent upon the activity of the one or more ribozymes.

95. The ribozyme RNA-construct(s) of claim 94, wherein the first and second ligation stem domains are from 30 to 60 bp in length.

96. The ribozyme RNA-construct(s) of claim 95, wherein the first and second ligation stem domains are from 40 to 50 bp in length.

97. The ribozyme RNA-construct(s) of claim 94, wherein the first and second ribozymes are selected from the group consisting of a twister ribozyme, a hammerhead ribozyme, a hatchet ribozyme, a hepatitis delta virus ribozyme, a ligase ribozyme, a pistol ribozyme, a twister sister ribozyme, a Vg1 ribozyme, a VS ribozyme and derivatives of any of the foregoing.

98. The ribozyme RNA-construct(s) of claim 97, wherein the first and second ribozymes are twister ribozymes.

99. The ribozyme RNA-construct(s) of claim 98, wherein the first ribozyme is a P3 twister ribozyme.

100. The ribozyme RNA-construct(s) of claim 98, wherein the second ribozyme is a P1 twister ribozyme.

101. The ribozyme RNA-construct(s) of claim 94, wherein the first ligation stem domain comprises a 5′-OH end, the second ligation stem domain comprises a 2′, 3′-cyclic phosphate end, and
wherein the first and second ligation stem domains form a stem substrate for an RNA ligase.

102. The ribozyme RNA-construct(s) of claim 101, wherein the RNA ligase is RtcB.

103. The ribozyme RNA-construct(s) of any one of claims 94-102, wherein the payload or the at least one polypeptide of interest comprises a zinc finger or CRISPR-Cas9 coding sequence.

104. The ribozyme RNA-construct(s) of any one of claim 94-102, wherein the payload domain or the at least one polypeptide of interest comprise a sequence that encodes a polypeptide/protein selected from insulin, clotting factor IX, the cystic fibrosis transmembrane conductance regulator protein, and the dystrophin protein.

105. The ribozyme RNA-construct(s) of any one of claims 94-102, wherein the ribozyme RNA-construct(s) is linearized.

106. The ribozyme RNA-construct(s) of any one of claims 94-105, wherein the 3′ UTR comprises a Woodchuck Hepatitis Virus Postranslational Regulatory Element (WPRE).

107. The ribozyme activated RNA construct(s) of claim 106, wherein the WPRE is followed by a poly(A) stretch.

108. The ribozyme activated RNA construct(s) of any one of claims 94-105, wherein the 3′ UTR sequence is selected from the group consisting of an mtRNR1-AES 3′ UTR (SEQ ID NO: 1354), an mtRNR1-LSP1 3′ UTR (SEQ ID NO: 1355), an AES-mtRNR1 3′ UTR (SEQ ID NO: 1356), an AES-hBg 3′ UTR (SEQ ID NO: 1357), an FCGRT-hBg 3′ UTR (SEQ ID NO: 1358), a 2hBg 3′ UTR (SEQ ID NO: 1359), and a HBA1 3′ UTR (SEQ ID NO: 1360), wherein the T nucleotides are U nucleotides in the RNA construct.

109. The ribozyme activated RNA construct(s) of any one of claims 94-108, wherein the first and/or the second ligation stem domain comprises a nucleic acid sequence having at least 90% sequence identity of 5′ AACCAUGCCGACUGAUGGCAG 3′ (SEQ ID NO: 1413).

110. The ribozyme activated RNA construct(s) of any one of claims 94-108, wherein the first and/or the second ligation stem domain comprises a nucleic acid sequence having at least 90% sequence identity of 5′ CUGCCAUCAGUCGGCGUGGACUGUAG 3′ (SEQ ID NO: 1415).

111. The ribozyme activated RNA construct(s) of claim 106, wherein the WPRE sequence comprises the nucleic acid sequence of SEQ ID NO: 1353.

112. The ribozyme activated RNA construct(s) of any one of claims 94-111, wherein the IRES sequence is selected from the group consisting of a cricket paralysis virus IRES (SEQ ID NO: 1330), a Homo sapiens IGF2 IRES (SEQ ID NO: 1331), a hepatovirus A IRES (SEQ ID NO: 1332), a hepatitis C virus H77 isolate IRES (SEQ ID NO: 1333), a Homo sapiens FGF1 IRES (SEQ ID NO: 1334), a bovine viral diarrhea virus 1 IRES (SEQ ID NO: 1335), a human rhinovirus A89 IRES (SEQ ID NO: 1336), a pan paniscus LIMA1 (SEQ ID NO: 1337), a human adenovirus 2 IRES (SEQ ID NO: 1338), a Montana myotis leukoencephalitis virus IRES (SEQ ID NO: 1339), a Homo sapiens RANBP3 IRES (SEQ ID NO: 1340), a pestivirus giraffe 1 IRES (SEQ ID NO: 1341), a Homo sapiens TGIF1 IRES (SEQ ID NO: 1342), a human poliovirus 1 mahoney IRES (SEQ ID NO: 1343), a foot-and-mouth disease virus type O IRES (SEQ ID NO: 1344), an encephalomyocarditis virus 7A IRES (SEQ ID NO: 1345), an encephalomyocarditis virus 6A IRES (SEQ ID NO: 1346), an enterovirus 71 IRES (SEQ ID NO: 1347), and a coxsackievirus B3 IRES (SEQ ID NO: 1348), wherein the T nucleotides are U nucleotides in the RNA construct.

113. The ribozyme RNA-construct(s) of any one of claims 94-112, wherein a vector or plasmid comprises the ribozyme RNA-construct(s) located downstream of an RNA promoter.

114. The ribozyme RNA-construct(s) of claim 113, wherein the RNA promoter is a polymerase III promoter.

115. The ribozyme RNA-construct(s) of claim 114, wherein the polymerase III promoter is a hU6 promoter.

116. The ribozyme RNA-construct(s) of claim 94, wherein the first and second ligation stem domains are substrates of naturally occurring ligases in situ.

117. The ribozyme RNA-construct(s) of claim 116, wherein the naturally occurring ligase is RtcB.

118. The ribozyme RNA-construct(s) of claim 94, wherein the at least one polypeptide of interest comprises two or more polypeptides of interest separated by a self-cleaving peptide.

119. The ribozyme RNA-construct(s) of claim 118, wherein the self-cleaving peptide comprises a 2A- or 2A-like-peptide.

120. The ribozyme RNA-construct(s) of claim 94, wherein the at least one polypeptide of interest is selected from the group consisting of a prodrug activating enzyme, a biological response modifier, a receptor ligand, an immunoglobulin derived binding polypeptide, a non-immunoglobulin binding polypeptide, an antigenic polypeptide, a genome editing enzyme, and any combination thereof wherein multiple polypeptides are separated by a 2A or 2A-like peptide.

121. The ribozyme RNA-construct(s) of claim 120, wherein the biological response modifier or an immunopotentiating cytokine.

122. The ribozyme RNA-construct(s) of claim 121, wherein the immunopotentiating cytokine is selected from the group consisting of interleukins 1 through 38, interferon, tumor necrosis factor (TNF), and granulocyte-macrophage-colony stimulating factor (GM-CSF).

123. The ribozyme RNA-construct(s) of claim 120, wherein the 2A- or 2A-like peptide further comprises a GSG linker moiety.

124. The ribozyme RNA-construct(s) of claim 120, wherein the genome editing enzyme is selected from the group consisting of a zinc finger nuclease, a transcription activator-like effector nuclease (TALEN), an engineered meganuclease and an RNA-guided DNA endonuclease (Cas) polypeptide.

125. The ribozyme RNA-construct(s) of any one of claims 35-53 and 94-124, wherein the 5′ and 3′ ribozyme sequences are independently selected from a sequence that is at least 85-100% identical to 5′-GCCATCAGTCGCCGGTCCCAAGCCCGGATAAAATGGGAGGGGGCGGGAAACCGC CT-3′ (SEQ ID NO:1349) or 5′-AACACTGCCAATGCCGGTCCCAAGCCCGGATAAAAGTGGAGGGTACAGTCCACG C-3′ (SEQ ID NO:1350), wherein T can be U.

126. The ribozyme RNA-construct(s) of any one of claims 35-53 and 94-124, wherein the first and second ligation stem domains are independently selected from a sequence that is at least 85-100% identical to 5′-AACCATGCCGACTGATGGCAG-3′ (SEQ ID NO:1351) or 5′-CTGCCATCAGTCGGCGTGGACTGTAG-3′ (SEQ ID NO:1352).

127. An RNA or DNA vector comprising the ribozyme RNA-construct(s) of any one of claims 35-53 and 94-126, the ribozyme RNA-construct(s) of any one of claims 35-53 and 94-126 or the DNA construct of any one of claims 54-71.

128. The vector of claim 127, wherein the vector is a viral vector.

129. The vector of claim 128, wherein the viral vector is a replicating or non-replicating retroviral vector.

130. The vector of claim 128, wherein the viral vector is an adenoviral vector, an adeno-associated viral vector (AAV), or a lentiviral vector.

131. The vector of claim 130, wherein the adenoviral vector is selected from the group consisting of AAV1 serotype, an AAV2 serotype, AAV3 serotype, an AAV4 serotype, AAV5 serotype, an AAV6 serotype, AAV7 serotype, an AAV8 serotype, an AAV9 serotype, a derivative of any of these.

132. A circular RNA construct obtain by in vitro transcription of a DNA construct of any one of claims 54-71 and a ribozyme activated RNA-construct of any one of claims 94-126.

133. The circular RNA construct of claim 132, wherein the construct comprises a duplex of the first and second ligation stem domains and (i) an internal ribosome entry site (IRES) or a P2A peptide coding sequence, (ii) a coding sequence of at least one polypeptide and/or nucleic acid of interest, and (iii) a 3′UTR sequence.

134. The circular RNA construct of claim 132, wherein the at least one polypeptide of interest is selected from the group consisting of a prodrug activating enzyme, a biological response modifier, a receptor ligand, an immunoglobulin derived binding polypeptide, a non-immunoglobulin binding polypeptide, an antigenic polypeptide, a genome editing enzyme, and any combination thereof wherein multiple polypeptides are separated by a 2A or 2A-like peptide.

135. The circular RNA construct of claim 129, wherein the circular RNA construct comprises a coding region for a gene editing polypeptide and a nucleic acid guide sequence.

136. A pharmaceutical composition comprising the RNA construct of any one of claims 1-15, 35-71, and 94-126, the vector of any one of claims 127-131 or the circular RNA construct of any one of claims 132-135, and a pharmaceutically acceptable carrier.

137. A host cell comprising the RNA construct of any one of claims 1-15, 35-71, and 94-126, the vector of any one of claims 127-131 or the circular RNA construct of any one of claims 132-135.

138. The host cell of claim 137, wherein the host cell is a eukaryotic cell.

139. The host cell of claim 137, wherein the ribozyme RNA construct, the vector or the circular RNA construct is episomal.

140. The host cell of claim 137, wherein the circular RNA constructs edits the genome or an expressed RNA in the host cell.

141. A vaccine composition comprising the ribozyme RNA-construct(s) of claim 90, wherein the ribozyme RNA-construct(s) is linearized and comprises:
a 5′ ribozyme;
a 5′ ligation sequence;
an internal ribosome entry site (IRES) sequence;
an RNA coding sequence for at least one antigenic polypeptide;
a 3′UTR sequence;
a 3′ ligation sequence; and
a 3′ ribozyme sequence, and a pharmaceutically acceptable carrier.

142. A vaccine composition comprising the RNA construct of claim 141, wherein the coding sequence encoding a polypeptide of interest encodes for an antigenic polypeptide.
